(19) United States
(12) Patent Application Publication
(10) Pub. No.: US 2022/0089599 A1
(43) Pub. Date: Mar. 24, 2022

(54) ALKYNYL NICOTINAMIDE COMPOUNDS AS KINASE INHIBITORS

(71) Applicant: PURDUE RESEARCH FOUNDATION, WEST LAFAYETTE, IN (US)

(72) Inventors: HERMAN O. SINTIM, WEST LAFAYETTE, IN (US); ELIZABETH LAROCQUE, WEST LAFAYETTE, IN (US); N. NAGANNA, WEST LAFAYETTE, IN (US)

(21) Appl. No.: 17/274,950

(22) PCT Filed: Sep. 12, 2019
(86) PCT No.: PCT/IB2019/057711

§ 371 Date: Mar. 10, 2021

Related U.S. Application Data
(60) Provisional application No. 62/730,046, filed on Sep. 12, 2018.

(51) Int. Cl. C07D 487/04; A61P 35/02; C07D 471/04

(52) U.S. Cl. CPC C07D 487/04; C07D 471/04; A61P 35/02

(57) ABSTRACT

This invention relates to Alkynyl Nicotinamide compounds and their derivatives as kinase inhibitors. In addition, uses thereof for treatment of disease such as cancer and, in particular, acute myeloid leukemia are disclosed.

### CROSS-REFERENCE TO RELATED APPLICATION

This application claims priority to U.S. Provisional Patent Application No. 62/730,046 filed Sep. 12, 2018. The entire contents and disclosures of this patent application are incorporated herein by reference.

### BACKGROUND

In human cells there are over 500 kinases regulating important processes, such as cell cycle regulation, proliferation, apoptosis and migration. Inhibitors of protein kinases have the potential to treat many diseases that are controlled by dis-regulation of protein kinases. Thus far over twenty kinase inhibitors have been approved by the FDA to treat various diseases.

Ponatinib, developed by Ariad pharmaceuticals as a multi-kinase inhibitor was approved by the Food and Drug Administration (FDA) in 2012¹. It currently targets many of the various cancer-driver kinases. This includes kinases such as ABL1, FLT3, FGFR1-4, and RET. Due to its impressive kinase inhibition profile it has been shown to potently inhibit various cancers, including CML, AML, various FGFR and RET-driven cancers (such as non-small cell lung cancer² and thyroid cancer³). Currently, ponatinib is the only FDA approved drug for imatinib-resistant CML that harbor the T315I mutation⁴. It is also undergoing various clinical trials for AML, lung and other cancers (NCT02428543; Ponatinib for FLT3-ITD Acute Myelogenous Leukemia (PONATINIB-AML)⁵, NCT02265341; Advanced Biliary Cancer with FGFR2 Fusions⁶, NCT01813734; Ponatinib in advanced NSCLC with RET Translocations⁷).

Despite these impressive arrays of cancer types that ponatinib is currently being evaluated against, the drug is relatively toxic and is associated with cardiovascular adverse events⁸. Patients taking Ponatinib have also shown side effects of hypertension, platelet dysfunction and peripheral arterial occlusive disease⁹. Other more serious side effects such as myocardial infraction, stroke, and liver failure have occurred in patients taking ponatinib¹⁰. Additionally, about 40% of patients on ponatinib developed some form of thrombosis. The FDA temporarily halted the sale of ponatinib in 2014 due to this adverse issue and it is now given as a drug of last resort for CML patients who have ABL (T315I) mutation and have not responded to any other therapy. See Gainor, J. F. et al., Ponatinib: Accelerated Disapproval, Oncologist, 20(8), 847-848 (2015); Talbert, D. R. et al., Toxicol. Sci, 143 (1), 147-155 (2015). The unfavorable toxicity profile associated with ponatinib could be due to the simultaneous inhibition of cardiovascular-related kinases¹¹.

In the efforts to develop kinase inhibitors against several disease-related kinases, it is discovered that 4-substituted isoquinolines are privileged kinases inhibitors. Further, the substitution pattern of these 4-substituted isoquinolines play critical roles in kinase selectivity and hence cancer selectivity. 4-Alkynyl-substituted aminoisoquinolines in particular have shown exceptional activity against various kinases and potently inhibit cancer proliferation. This important discovery has facilitated the tailoring of 4-substituted aminoisoquinoline into compounds that inhibit various cancers. Additionally, the 4-alkynyl-substituted 1- or 3-amino isoquinolines can be tuned for selectivity and toxicity and hence represent a new-generation alkyne-containing kinase inhibitors with desirable drug-like properties. See U.S. application Ser. No. 16/325,022, filed Aug. 15, 2017. The entire contents and disclosures of this patent application are incorporated herein by reference.

### SUMMARY

According to first broad aspect, the present invention we provide a nicotinamide analog of ponatinib, whereby the benzamide moiety in ponatinib is replaced with a nicotinamide analog, could be a better and less toxic alternative to ponatinib.

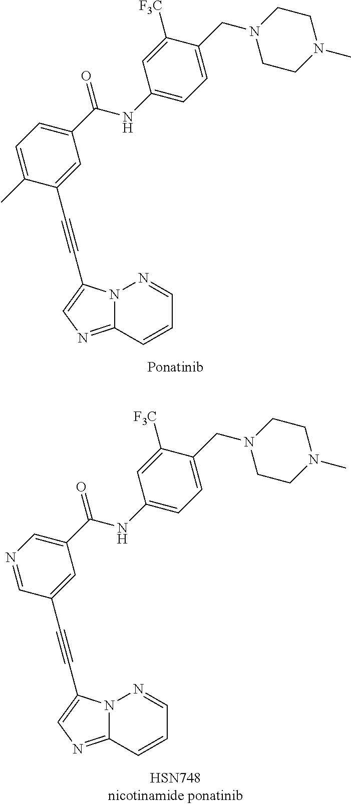

In another aspect, the present invention is directed to a pharmaceutical composition comprising one or more compounds as described herein, or a pharmaceutically acceptable salt, N-oxide, hydrate, solvate, tautomer, or optical isomer thereof, and a pharmaceutically acceptable carrier or diluent.

In yet another aspect, the present invention is directed to a method of treating, inhibiting, suppressing, or reducing the severity of a disease or a disorder associated with protein kinase in a subject in need thereof, wherein the method comprises administering to the subject a therapeutically effective amount of a compound as described herein, or a pharmaceutically acceptable salt, N-oxide, hydrate, solvate, tautomer, or optical isomer thereof, or a pharmaceutical composition containing one or more compounds as described herein.

The details of one or more embodiments of the invention are set forth in the accompanying the description below. Other features, objects, and advantages of the invention will be apparent from the description and drawings, and from the claims.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a schematic illustration showing the replacement of methyl benzamide with nicotinamide moiety in Pontainib according to one embodiment of the present invention.

FIG. 2 is a diagram showing the general structure of the inventive compounds according to one embodiment of the present invention.

FIG. 3 is a diagram showing the structure of the group 1 inventive compounds according to one embodiment of the present invention.

FIG. 4 is a diagram showing the structure of the group 2 inventive compounds according to one embodiment of the present invention.

FIG. 5 is a diagram showing the structure of the group 3 inventive compounds according to one embodiment of the present invention.

FIG. 6 is a diagram showing the structure of the group 4 inventive compounds according to one embodiment of the present invention.

FIGS. 7A and 7B are diagrams showing the structure of the group 5 inventive compounds according to one embodiment of the present invention.

FIG. 8 is a diagram showing the structure of the group 6 inventive compounds according to one embodiment of the present invention.

FIG. 9 is a diagram showing the structure of the group 7 inventive compounds according to one embodiment of the present invention.

FIG. 10 is a diagram showing the structure of the group 8 inventive compounds according to one embodiment of the present invention.

FIG. 11 is a diagram showing the structure of the group 9 inventive compounds according to one embodiment of the present invention.

FIG. 12 is a diagram showing the structure of the group 10 inventive compounds according to one embodiment of the present invention.

FIG. 13 is a diagram showing the structure of the group 11 inventive compounds according to one embodiment of the present invention.

FIG. 14 is a diagram showing the structure of the group 12 inventive compounds according to one embodiment of the present invention.

FIG. 15 is a diagram showing the structure of the group 13 inventive compounds according to one embodiment of the present invention.

FIG. 16 is a diagram showing the structure of the group 14 inventive compounds according to one embodiment of the present invention.

FIG. 17 is a diagram showing the structure of the group 15 inventive compounds according to one embodiment of the present invention.

FIG. 18 is a diagram showing the structure of the group 16 inventive compounds according to one embodiment of the present invention.

FIG. 19 is a diagram showing the structure of the group 17 inventive compounds according to one embodiment of the present invention.

FIG. 20 is a diagram showing the structure of the group 18 inventive compounds according to one embodiment of the present invention.

FIG. 21 is a diagram showing the structure of the group 19 inventive compounds according to one embodiment of the present invention.

FIG. 22 is a diagram showing the structure of the group 20 inventive compounds according to one embodiment of the present invention.

FIG. 23 is a diagram showing the structure of the group 21 inventive compounds according to one embodiment of the present invention.

FIG. 24 is a diagram showing the structure of the group 22 inventive compounds according to one embodiment of the present invention.

FIG. 25 is a diagram showing the structure of the group 23 inventive compounds according to one embodiment of the present invention.

FIG. 26 is a diagram showing the structure of the group 24 inventive compounds according to one embodiment of the present invention.

FIG. 27 is a diagram showing the structure of the group 25 inventive compounds according to one embodiment of the present invention.

FIG. 28 is a diagram showing the structure of the group 26 inventive compounds according to one embodiment of the present invention.

FIG. 29 is a diagram showing the structure of the group 27 inventive compounds according to one embodiment of the present invention.

FIG. 30 is a schematic illustration of a treatment delivery apparatus according to one embodiment of the present invention.

### DETAILED DESCRIPTION OF THE INVENTION

### Definitions

Unless defined otherwise, all technical and scientific terms used herein have the same meaning as is commonly understood to which the claimed subject matter belongs. If there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present invention, the term “comprising”, the term “having”, the term “including,” and variations of these words are intended to be open-ended and mean that there may be additional elements other than the listed elements.

For purposes of the present invention, directional terms such as “top,” “bottom,” “upper,” “lower,” “above,” “below,” “left,” “right,” “horizontal,” “vertical,” “up,” “down,” etc., are used merely for convenience in describing the various embodiments of the present invention. The embodiments of the present invention may be oriented in various ways. For example, the diagrams, apparatuses, etc., shown in the drawing figures may be flipped over, rotated by 90° in any direction, reversed, etc.

For purposes of the present invention, a value or property is “based” on a particular value, property, the satisfaction of a condition, or other factor, if that value is derived by performing a mathematical calculation or logical decision using that value, property or other factor.

For purposes of the present invention, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present invention, the term “analogue” and the term “analog” refer to one of a group of chemical compounds that share structural and/or functional similarities but are different in respect to elemental composition. A structural analog is a compound having a structure similar to that of another one, but differing from it in respect of one or more components, such as one or more atoms, functional groups, or substructures, etc. Functional analogs are compounds that has similar physical, chemical, biochemical, or pharmacological properties. Functional analogs are not necessarily also structural analogs with a similar chemical structure.

For purposes of the present invention, the term “ameliorate” and the term “amelioration” to any lessening of severity, delay in onset, slowing of progression, or shortening of duration, whether permanent or temporary, lasting or transient of the symptoms of a particular disease, disorder or condition by administration of a drug or pharmaceutical composition.

For purposes of the present invention, the term “amino acid” refers to the molecules composed of terminal amine and carboxylic acid functional groups with a carbon atom between the terminal amine and carboxylic acid functional groups sometimes containing a side chain functional group attached to the carbon atom (e.g. a methoxy functional group, which forms the amino acid serine). Typically, amino acids are classified as natural and non-natural. Examples of natural amino acids include glycine, alanine, valine, leucine, isoleucine, proline, phenylananine, tyrosine, tryptophan, serine, threonine, cysteine, methionine, asparagine, glutamine, lysine, arginine, histidine, aspartate, and glutamate, among others. Examples of non-natural amino acids include L-3,4-dihydroxyphenylalanine, 2-aminobutyric acid, dehydralanine, g-carboxyglutamic acid, carnitine, gamma-aminobutyric acid, hydroxyproline, and selenomethionine, among others. In the context of this specification it should be appreciated that the amino acids may be the L-optical isomer or the D-optical isomer.

For purposes of the present invention, the term “analyte” refers to the conventional meaning of the term “analyte,” i.e., a substance or chemical constituent of a sample that is being detected or measured in a sample. In one embodiment of the present invention, a sample to be analyzed may be an aqueous sample, but other types of samples may also be analyzed using a device of the present invention.

For purposes of the present invention, the term “antagonist” refers to a compound that binds to a receptor and blocks or disrupts the action of an agonist at the receptor.

For purposes of the present invention, the term “biomolecule” refers to the conventional meaning of the term biomolecule, i.e., a molecule produced by or found in living cells, e.g., a protein, a carbohydrate, a lipid, a phospholipid, a nucleic acid, etc.

For purposes of the present invention, the term “capsule” refers to a gelatinous envelope enclosing an active substance. Capsules may be soft-shelled capsules (softgels) or hard-shelled capsules. Capsules can be designed to remain intact for some hours after ingestion in order to delay absorption. They may also contain a mixture of slow- and fast-release particles to produce rapid and sustained absorption in the same dose.

For purposes of the present invention, the term “carrier” refers to relatively nontoxic chemical compounds or agents that facilitate the incorporation of a drug into cells or tissues.

For purposes of the present invention, the term “co-administration” refers to administration of two or more compositions or compounds to a single subject. Each of the two or more compositions may be administered by the same or different route of administration, at the same time or different time. Co-administration of first therapeutically effective compound and a second therapeutically effective compound, which for example, may be dissolved or intermixed in the same pharmaceutically acceptable carrier.

For purposes of the present invention, the term “combination” refers to both a “fixed-dose combination” or a “co-packaged drug products.” A “fixed-dose combination” or a “fixed combination” is a formulation that includes two or more active pharmaceutical ingredients, e.g., medicaments, compounds, physically combined in a single dosage form. In another words, medicaments or compounds may be dissolved or intermixed in a same pharmaceutically acceptable carrier. The form of a single dosage can be, but is not limited to, a tablet, a softgel, a capsule, a hard capsule, a caplet, a chewable tablet, a gummy, an injection fluid, a transdermal patch, etc. A “combination product” refers to a product that combines drugs, devices, and/or biological products. Sometimes, a combination product may be a polypill or a combo pill in the dosage form such as a tablet, a capsule, etc. Sometimes, a “combination product” may a “non-fixed combination” or a “co-packaged drug product” in which two or more separate dosage forms packaged together in a single package or as a unit. Drug, device, or biological product may be packaged separately according to specific needs such as proposed labeling. The contents of a “non-fixed combination” may be administered to a subject simultaneously, concurrently, or sequentially at different time intervals or with no specific intervening time limits, wherein such administration provides effective levels of the medicaments or compounds in the body of the subject. A “combination administration” includes co-administration of various compounds in therapeutically effective amount, wherein the various compounds may be in a “fixed-dose combination” or in a “non-fixed combination.” A “concurrent administration” includes the administration of various compounds separately at the same time or sequentially in any order at different points in time to provide an effect suitable for the treatment. Therapy being either concomitant or sequential may be dependent on the characteristics of the other medicaments or compounds used, characteristics like onset and duration of action, plasma levels, clearance, etc.

For purposes of the present invention, the term “controlled release” refers to time dependent release. Timed release has several distinct variants such as sustained release where prolonged release is intended, pulse release, delayed release, etc. Time dependent release may be in oral dose formulations such as pills, capsules, gels, and may also in formulations such as injectable drug carriers, implants, and devices, and transdermal patches.

For purposes of the present, the term “delayed release” refers to oral medicines that do not immediately disintegrate and release the active ingredient(s) into the body. For example, an enteric coated oral medication dissolves in the intestines rather than the stomach.

For purposes of the present invention, the term “dietary supplement” refers to a product taken by mouth that contains a “dietary ingredient” intended to supplement the diet. The “dietary ingredients” in these products may include vitamins, minerals, herbs or other botanicals, amino acids, and substances such as enzymes and metabolites. Dietary supplements may also be extracts or concentrates and may be found in many dosage forms such as tablets, hard capsules, softgels, chewable tablets, gummies, liquids, or powders. Dietary supplements may also be in other dosage forms, such as a bar, but if they are, information on the label of the dietary supplement may not represent the product as a conventional food or a sole item of a meal or diet.

For purposes of the present invention, the term “diagnosing” refers to identify the nature of a disease, illness, condition or other problem by examination of the symptoms in an individual or patient.

For purposes of the present invention, the term “diluent” refers to a chemical compound that is used to dilute a drug prior to delivery. Diluents can also be used to stabilize agents because they can provide a more stable environment. Pharmaceutically acceptable salt dissolved in buffered solutions (which also can provide pH control or maintenance) are utilized as diluents in the art, including, but not limited to a phosphate buffered saline solution.

For purposes of the present invention, the term “dosage” refers to the administering of a specific amount, number, and frequency of doses over a specified period. Dosage implies duration. A “dosage regimen” is a treatment plan for administering a drug over a period.

For purposes of the present invention, “dosage form” and the term “unit dose” refer to an individual dose of a pharmaceutical product. Dosage forms may comprise a mixture of active drug components and nondrug components (excipients), along with other non-reusable material that may not be considered either ingredient or packaging.

For purposes of the present invention, the term “dose” refers to a specified amount of medication taken at one time.

For purposes of the present invention, the term “drug” refers to a material that may have a biological effect on a cell, including but not limited to small organic molecules, inorganic compounds, polymers such as nucleic acids, peptides, saccharides, or other biologic materials, nanoparticles, etc.

For purposes of the present invention, the term “effective amount” or “effective dose” or grammatical variations thereof refers to an amount of an agent sufficient to produce one or more desired effects. The effective amount may be determined by a person skilled in the art using the guidance provided herein.

For purposes of the present invention, the term “enhance” and the term “enhancing” refer to increasing or prolonging either in potency or duration of a desired effect. By way of example, “enhancing” the effect of therapeutic agents singly or in combination refers to the ability to increase or prolong, either in potency, duration and/or magnitude, the effect of the agents on the treatment of a disease, disorder or condition. When used in a patient, amounts effective for this use will depend on the severity and course of the disease, disorder or condition, previous therapy, the patient's health status and response to the drugs, and the judgment of the treating physician.

For purposes of the present invention, the term “enteric coating” refers to a polymer barrier applied on oral medication.

For purposes of the present invention, the term “fluid” refers to a liquid or a gas.

For purposes of the present invention, the term “individual” refers to an individual mammal, such as a human being.

For purposes of the present invention, the term “ligand” refers to a substance, such as a small molecule, that forms a complex with a biomolecule to serve a biological purpose. In protein-ligand binding, the ligand is usually a signal-triggering molecule, binding to a site on a target protein. Ligand binding to a receptor protein (receptor) alters the receptor's chemical conformation (three-dimensional shape). The conformational state of a receptor determines its functional state.

Ligands include substrates, inhibitors, activators, and neurotransmitters.

For purposes of the present invention, the term “lipid” refers to hydrophobic or amphiphilic molecules, including but not limited to biologically derived lipids such as phospholipids, triacylglycerols, fatty acids, cholesterol, or synthetic lipids such as surfactants, organic solvents, oils, etc.

For purposes of the present invention, the term “long-chain fatty acid” refers to a fatty acid having an aliphatic tail of 13 or more carbon atoms.

For purposes of the present invention, the term “long-chain fatty acid group” refers to the ester group derived from a long-chain fatty acid. An example of a long-chain fatty acid group is a stearate group.

For purposes of the present invention, the term “medical therapy” refers to prophylactic, diagnostic and therapeutic regimens carried out in vivo or ex vivo on humans or other mammals.

For purposes of the present invention, the term “mg/kg” refers to the dose of a substance administered to an individual or a subject in milligrams per kilogram of body weight of the individual or the subject.

For purposes of the present invention, the term “nutraceutical” refers to compounds and compositions that are useful in both the nutritional and pharmaceutical field of application. Thus, nutraceutical compositions of the present invention may be used as supplement to food and beverages, and as pharmaceutical formulations for enteral or parenteral application which may be solid formulations such as capsules or tablets, or liquid formulations, such as solutions or suspensions. In some embodiments of the present invention, nutraceutical compositions may also comprise food and beverages containing therapeutically effective amount of one or more respective selective dopamine D4 receptor agonists and/or pharmaceutically acceptable analogs, pharmaceutically acceptable salts or hydrates of the one or more respective selective dopamine D4 receptor agonists, as well as supplement compositions, for example dietary supplements.

For purposes of the present invention, the term “parenteral route” refers to the administration of a composition, such as a drug in a manner other than through the digestive tract. Parenteral routes include routes such as intravenous, intra-arterial, transdermal, intranasal, sub-lingual and intraosseous, etc. For example, intravenous is also known as I.V., which is giving directly into a vein with injection. As the drug directly goes into the systemic circulation, it reaches the site of action resulting in the onset the action.

For purposes of the present invention, the term “patient” and the term “subject” refer to a mammal, animal, fish, reptile, avian or which is the object of treatment, observation or experiment. By way of example only, a subject may be, but is not limited to, a mammal including, but not limited to, a human.

For purposes of the present invention, the term “pharmaceutically acceptable” refers to a compound or drug approved or approvable by a regulatory agency of a federal or a state government, listed or listable in the U.S. Pharmacopeia or in other generally recognized pharmacopeia for use in mammals, including humans.

For purposes of the present invention, the term “pharmaceutically acceptable carrier” refers to a carrier that comprises pharmaceutically acceptable materials. Pharmaceutically acceptable carriers include, but are not limited to, saline solutions and buffered solutions. Pharmaceutically acceptable carriers are described for example in Gennaro, Alfonso, Ed., Remington's Pharmaceutical Sciences, 18th Edition 1990. Mack Publishing Co., Easton, Pa., a standard reference text in this field. Pharmaceutical carriers may be selected in accordance with the intended route of administration and the standard pharmaceutical practice.

For purposes of the present invention, the term “pharmaceutically acceptable salt” refers to those salts of compounds that are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. They may be prepared in situ when finally isolating and purifying the compounds of the invention, or separately by reacting them with pharmaceutically acceptable non-toxic bases or acids, including inorganic or organic bases and inorganic or organic acids. Pharmaceutically acceptable salts may be obtained using standard procedures well known in the art, for example by mixing a compound of the present invention with a suitable acid, for instance an inorganic acid or an organic acid. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

For purposes of the present invention, the term “pharmaceutical composition” refers to a product comprising one or more active ingredients, and one or more other components such as carriers, stabilizers, diluents, dispersing agents, suspending agents, thickening agents, and/or excipients, etc. A pharmaceutical composition includes enough of the active object compound to produce the desired effect upon the progress or condition of diseases and facilitates the administration of the active ingredients to an organism. Multiple techniques of administering the active ingredients exist in the art including, but not limited to topical, ophthalmic, intraocular, periocular, intravenous, oral, aerosol, parenteral, and administration. By “pharmaceutically acceptable,” it is meant the carrier, diluent or excipient must be compatible with the other ingredients of the formulation and not deleterious to the recipient thereof, i.e., the subject.

For purposes of the present invention, the term “pharmaceutical formulation” and the term “drug formulation” refer to a mixtures or a structure in which different chemical substances, including the active drug, are combined to form a final medicinal product, such as a sterile product, a capsule, a tablet, a powder, a granule, a solution, an emulsion, a topical preparation, a non-conventional product such as semi-solid or sustained-release preparations, liquid, etc. Pharmaceutical formulation is prepared according to a specific procedure, a “formula.” The drug formed varies by the route of administration. For example, oral drugs are normally taken as tablet or capsules.

For purposes of the present invention, the term “polypill” refers to a drug product in pill form (i.e., tablet or capsule) that combines multiple active pharmaceutical ingredients. A polypill comprises multiplicity of distinct drugs in a given “pill.” It may be manufactured as a fixed-dose combination drug product.

For purposes of the present invention, the term “prophylactically effective amount,” refers to that amount of a drug, compound, agent, combination or pharmaceutical composition which will relieve in a patient to some extent one or more of the symptoms of a disease, condition or disorder being treated in the patient. In such prophylactic applications, such amounts may depend on the patient's state of health, weight, and the like. It is considered well within the skill of the art for one to determine such prophylactically effective amounts by routine experimentation, including, but not limited to, a dose escalation clinical trial.

For purposes of the present invention, the term “synergistic effect” refers to a combined effect when two or more substances or biological structures interact resulting in an overall effect that is greater than the sum of individual effects of any of the two or more substances or biological structures. For example, a synergistic effect of two therapeutic compounds means that an effect of administering two therapeutic compounds in combination is greater than the sum of each effect when each of the two therapeutic compounds is administered alone.

For purposes of the present invention, the term “tablet” refers to a pharmaceutical dosage form. A tablet comprises a mixture of active substances and excipients, usually in powder form, pressed or compacted from a powder into a solid dose. The excipients can include diluents, binders or granulating agents, glidants and lubricants to ensure efficient tableting; disintegrants to promote tablet break-up in the digestive tract; sweeteners or flavors to enhance taste; and pigments to make the tablets visually attractive. A polymer coating is often applied to make the tablet smoother and easier to swallow, to control the release rate of the active ingredient, to make it more resistant to the environment (extending its shelf life), or to enhance the tablet's appearance. The disintegration time can be modified for a rapid effect or for sustained release. For example, Some tablets are designed with an osmotically active core, surrounded by an impermeable membrane with a pore in it. This allows the drug to percolate out from the tablet at a constant rate as the tablet moves through the digestive tract. Tablets can also be coated with sugar, varnish, or wax to disguise the taste. A tablet in an embodiment of the present may comprise a tablet without or with one or more coatings. A tablet may also have one or more layers. A tablet may be mini tablet, a meltable table, chewable tablet, an effervescent tablet or an orally disintegrating tablet.

For purposes of the present invention, the term “target” refers to a living organism or a biological molecule to which some other entity, like a ligand or a drug, is directed and/or binds. For example, “target protein” may a biological molecule, such as a protein or protein complex, a receptor, or a portion of a biological molecule, etc., capable of being bound and regulated by a biologically active composition such as a pharmacologically active drug compound.

For purposes of the present invention, the term “time release,” the term “extended-release,” or “controlled-release” refers to prolong an absorption of drugs with short half-lives, thereby allowing longer dosing intervals while minimizing fluctuations in serum drug levels. For example, a drug in a time release pill tables or capsules drug may be dissolved over time and be released slower and steadier into the bloodstream while having the advantage of being taken at less frequent intervals than immediate-release formulations of the same drug.

For purposes of the present invention, the term “therapeutically effective amount” and the term “treatment-effective amount” refers to the amount of a drug, compound or composition that, when administered to a subject for treating a disease or disorder, or at least one of the clinical symptoms of a disease or disorder, is sufficient to affect such treatment of the disease, disorder, or symptom. A “therapeutically effective amount” may vary depending, for example, on the compound, the disease, disorder, and/or symptoms of the disease or disorder, severity of the disease, disorder, and/or symptoms of the disease or disorder, the age, weight, and/or health of the subject to be treated, and the judgment of the prescribing physician. An appropriate amount in any given instance may be readily ascertained by those skilled in the art or capable of determination by routine experimentation.

For purposes of the present invention, the term “transdermal patch” refers to a medicated adhesive patch that is placed on the skin to deliver a specific dose of medication through the skin and into the bloodstream. A transdermal patch may provide a controlled release of the medication into the body of a subject.

For purposes of the present invention, the term “treating” or the term “treatment” of any disease or disorder refers to arresting or ameliorating a naturally occurring condition (for example, as a result of aging), disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to slowing the progression of a condition, inhibiting the disease or disorder, either physically, (e.g., stabilization of a discernible symptom), physiologically, (e.g., stabilization of a physical parameter), or both, and to inhibiting or slowing the progression of at least one physical parameter which may or may not be discernible to the subject. In some embodiments of the present invention, the terms “treating” and “treatment” refer to delaying the onset of the progression of the disease or disorder or at least one or more symptoms thereof in a subject who may be exposed to or predisposed to a disease or disorder even though that subject does not yet experience or display symptoms of the disease or disorder. The term “treatment” as used herein also refers to any treatment of a subject, such as a human condition or disease, and includes: (1) inhibiting the disease or condition, i.e., arresting the development or progression of the disease or condition, (2) relieving the disease or condition, i.e., causing the condition to regress, (3) stopping the symptoms of the disease, and/or (4) enhancing the conditions desired.

For purposes of the present invention, the term “vehicle” refers to a substance of no therapeutic value that is used to convey an active medicine for administration.

For purposes of the present invention, the term “room temperature” refers to a temperature of from about 20° C. to about 25° C.

For purposes of the present invention, the term “sparingly soluble in water” refers to a substance having a solubility of 0.1 g per 100 ml of water to 1 g per 100 ml of water. Unless specified otherwise, the term “sparingly soluble” and “sparingly soluble in water” are used interchangeably in the description of the invention below to refer to substances that are sparingly soluble in water.

### DESCRIPTION

While the invention is susceptible to various modifications and alternative forms, specific embodiment thereof has been shown by way of example in the drawings and will be described in detail below. It should be understood, however that it is not intended to limit the invention to the particular forms disclosed, but on the contrary, the invention is to cover all modifications, equivalents, and alternatives falling within the spirit and the scope of the invention.

U.S. Pat. No. 8,114,874 B2 teaches us that substituted acetylenic imidazo-[1,2-B]pyridazine compounds, containing benzamide unit, are kinase inhibitors. Ponatinib, one of such compounds, is a multi-kinase inhibitor, which potently inhibits ABL1, FLT3, RET, c-Src, c-Kit, FGFR, VEGFR, PDGFRα, PDGFRβ□□ BRAF, and other kinases. Thus far, ponatinib has shown potent inhibition of cancers driven by various cell finest.¹⁻⁷

Ponatinib administration is associated with many adverse toxicities, partly due to the concurrent inhibitions of many essential kinases. Analogs of ponatinib with reduced inhibition of cardiovascular-related kinases, such as VEGFR1-3, c-Src, c-Kit etc. are predicted to exhibit lower adverse toxicities.⁸⁻¹¹

The mechanistic Target of Rapamycin (mTOR) is an important drug target as mTOR integrates many stimuli and coordinates the adaptive response of many cellular processes.¹² Rapamycin is an inhibitor of mTOR. The MAPK-interacting kinase (MNK) contributes to rapamycin resistance by sustaining mTORC1 activity upon rapamycin treatment in cancer cells.¹² Thus concurrent inhibition of MNK1 and/or MNK2 and any of cancer-driver kinases, such as FLT3, ABL1, RET, BRAF, c-Kit, PDGFRα, PDGFRβ, could lead to more sustained inhibition of cancer growth.

MNK1 and 2 modulates the function eIF4E (a key player in translational control, which is elevated in human cancers MNK1 and 2 phosphorylates a conserved serine (Ser209) of eIF4E to modulate function.¹² The inhibition of both MNK1 and 2 have been shown to lead to growth inhibition in cancers.¹²

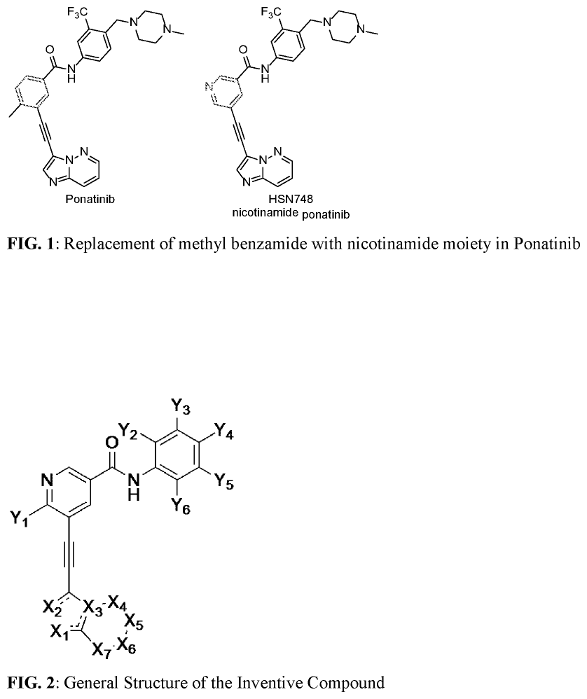

Ponatinib however does not potently inhibit MNK1 and MNK2, kinases that play important roles in cancer progression. However, we have discovered that the replacement of the benzamide group in ponatinib with a nicotinamide group results in new compounds, such as HSN748, which potently inhibit MNKs. Referring to FIG. 1, the replacement of methyl benzamide with nicotinamide moiety in Ponatinib is illustrated.

As described in FIG. 1, in the compounds of the invention, the length of the amide head group, substitution pattern and relative position to the alkyne moiety remarkably affects the anticancer activity against MV4-11 cell line (AML cell line). For example, the nature of the amide group in the molecules shown in FIG. 1 had a dramatic effect on the anticancer activities of the molecules tested.

Ponatinib potently inhibits FLT3-ITD but it has a weak activity against drug-resistant FLT3-D835Y and/or FLT3-ITD-D835Y. Thus, the secondary mutation FLT3-ITD-D835Y is a possible escape mechanism for acute myeloid leukemia treated with ponatinib. Nicotinamide analogs of ponatinib, such as HSN748 on the other hand are potent inhibitors of FLT3-ITD-D835Y. HSN748 inhibits Molm14(FLT3-ITD, D835Y) cell line about 100× more potently than ponatinib.

The differences between ponatinib and HSN748 are replacement of benzamide core in ponatinib with nicotinamide core and replacement of the methyl group on the benzamide of ponatinib with hydrogen. These two modifications lead to a compound, HSN748 and analogs, with reduced Log P (or reduced hydrophobicity). The calculated Log P changed from 4.47 (Ponatinib) to 3.44 (HSN748).¹³ Thus HSN748 has different drug properties to ponatinib.

Ponatinib is a potent inhibitor of platelet related c-Src in vitro whereas the nicotinamide version without a methyl group HSN748 is not a strong c-Src inhibitor in vitro. c-Src is important for platelet function hence the potent and sustained inhibition of c-Src could lead to adverse events which are avoided by the compounds described in this invention.

Based on the discovery of a nicotinamide version of ponatinib with different and desirable properties, the inventive compound having a general structure, as illustrated in FIG. 2 and below is provided:

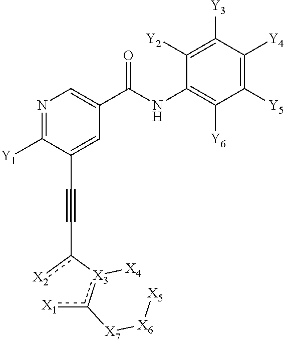

Where Y==amide; O-alkyl such as OMe, OEt, OPr, OBu, OiPr, OCF₂, OCF₃; NH₂, NH-alkyl, such as NHMe, NHEt; N-(alkyl)₂ or N-(heteroalkyl)₂, such as NMe₂, morpholino, piperazine; CN, Cl, Br, iPr, Et, cyclopropyl, butyl, CF₃, CHF₂, CH₂-piperazine analog, CH₂-morpholine analog, CH₂-piperidine analog, CH₂-pyrrolidine analog, CH₂-azetidine analog, etc., X1-X7=CH, CY or N. This general compound is hereinafter referred to as Formula (I).

In some embodiments, the compound of the invention is:

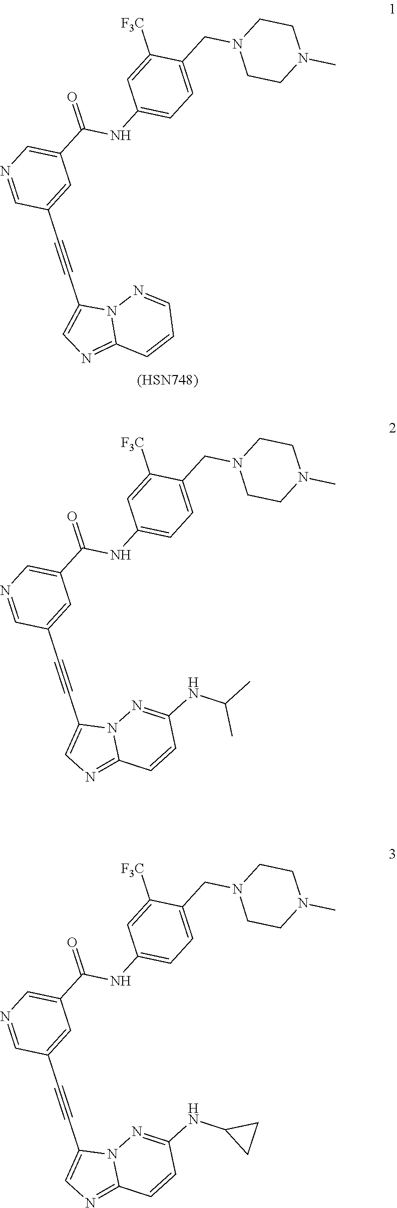

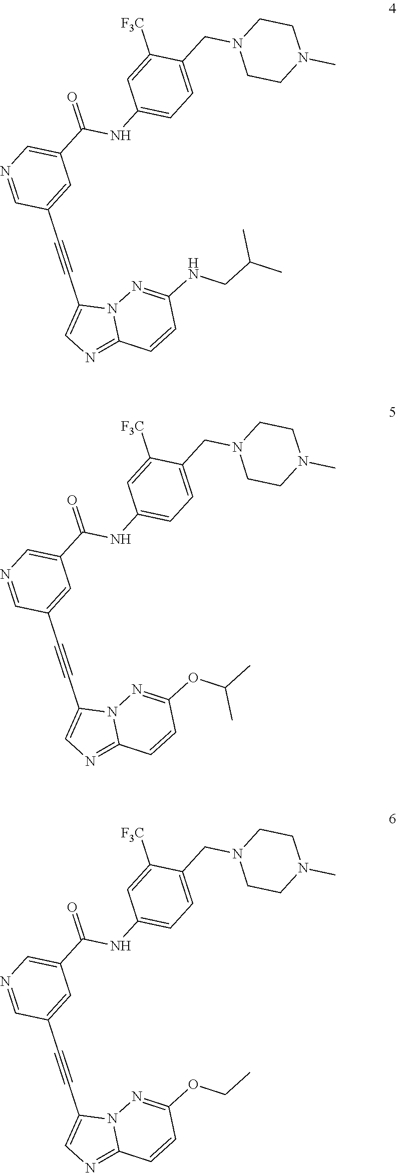 [SMILES: CC(C)CNc1ccc2ncc(C#Cc3cncc(C(=O)Nc4ccc(CN5CCN(C)CC5)c(C(F)(F)F)c4)c3)n2n1 ; CC(C)Oc1ccc2ncc(C#Cc3cncc(C(=O)Nc4ccc(CN5CCN(C)CC5)c(C(F)(F)F)c4)c3)n2n1 ; CCOc1ccc2ncc(C#Cc3cncc(C(=O)Nc4ccc(CN5CCN(C)CC5)c(C(F)(F)F)c4)c3)n2n1]

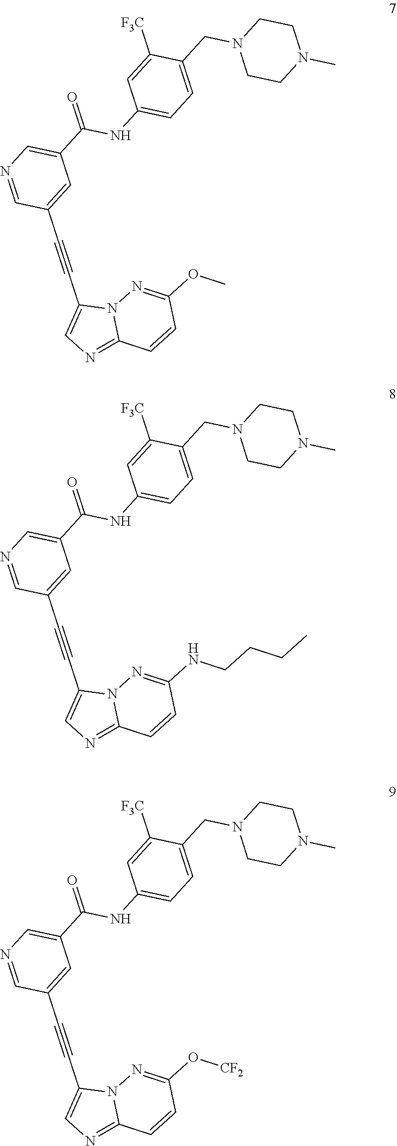

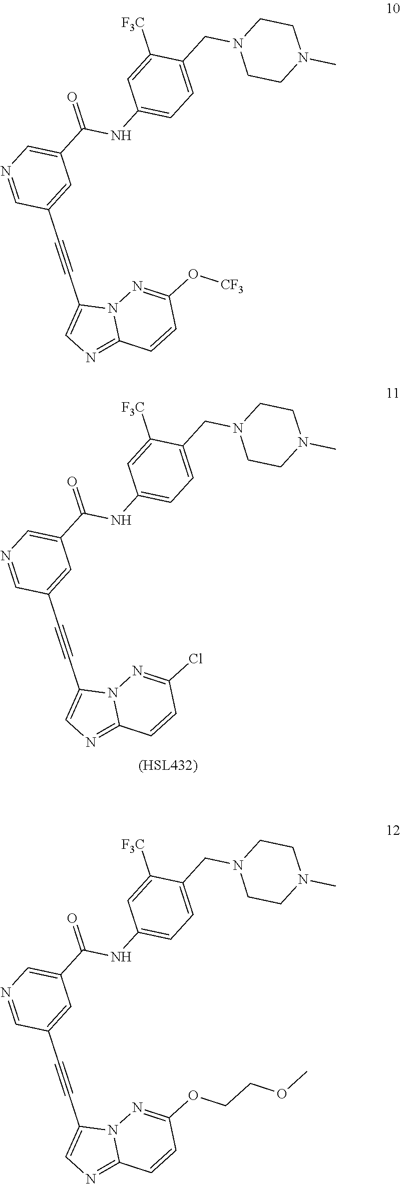 [SMILES: CN1CCN(Cc2ccc(NC(=O)c3cncc(C#Cc4cnc5ccc(OC(F)(F)F)nn45)c3)cc2C(F)(F)F)CC1 ; CN1CCN(Cc2ccc(NC(=O)c3cncc(C#Cc4cnc5ccc(Cl)nn45)c3)cc2C(F)(F)F)CC1 ; COCCOc1ccc2ncc(C#Cc3cncc(C(=O)Nc4ccc(CN5CCN(C)CC5)c(C(F)(F)F)c4)c3)n2n1]

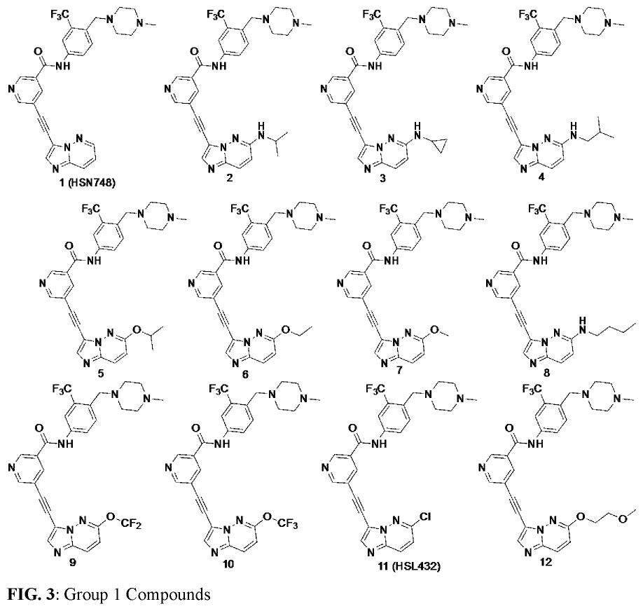

These compounds are hereinafter referred to as Group 1 compounds and are illustrated in FIG. 3.

In some embodiments, the compound of the invention is:

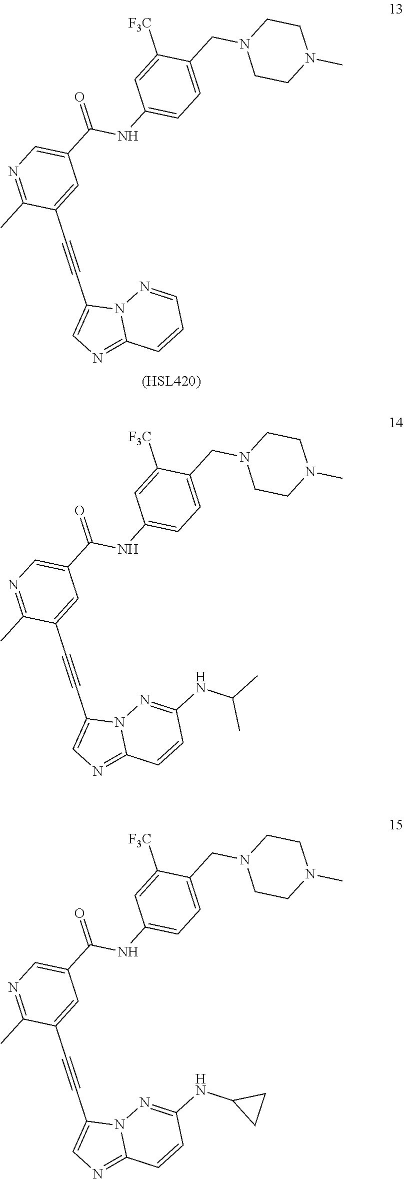

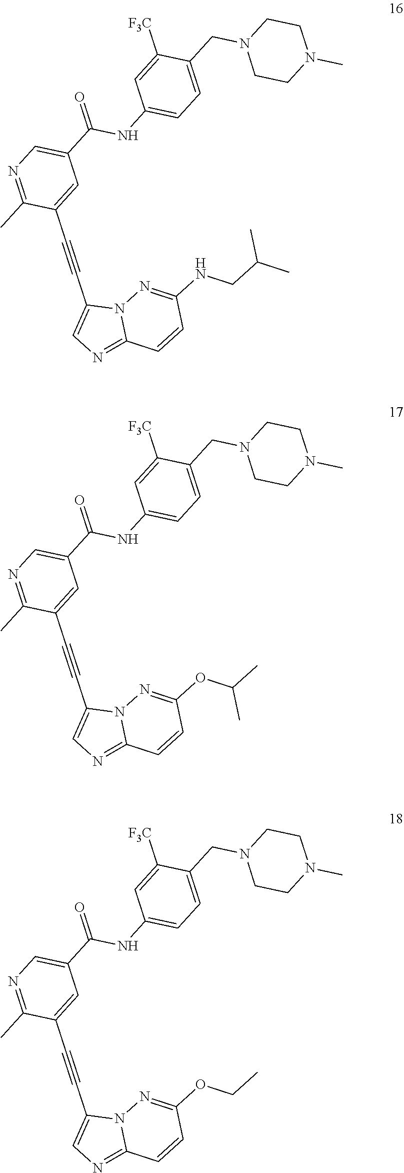

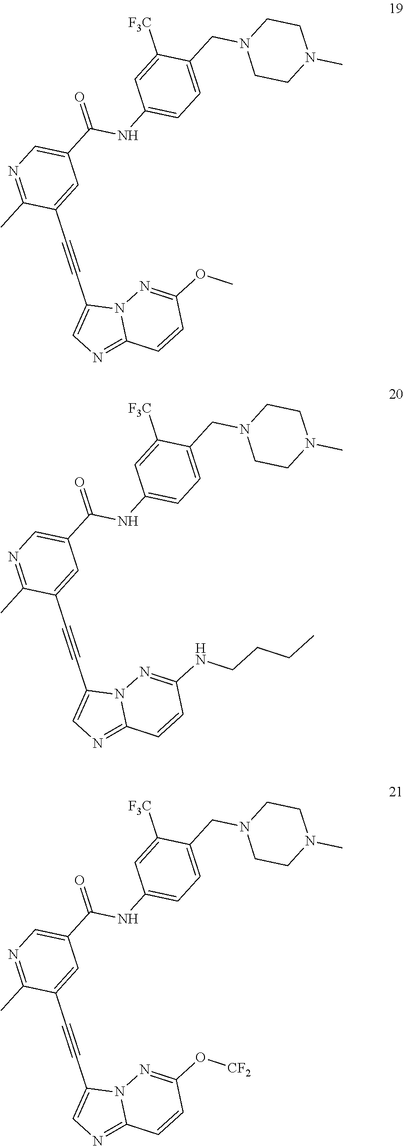

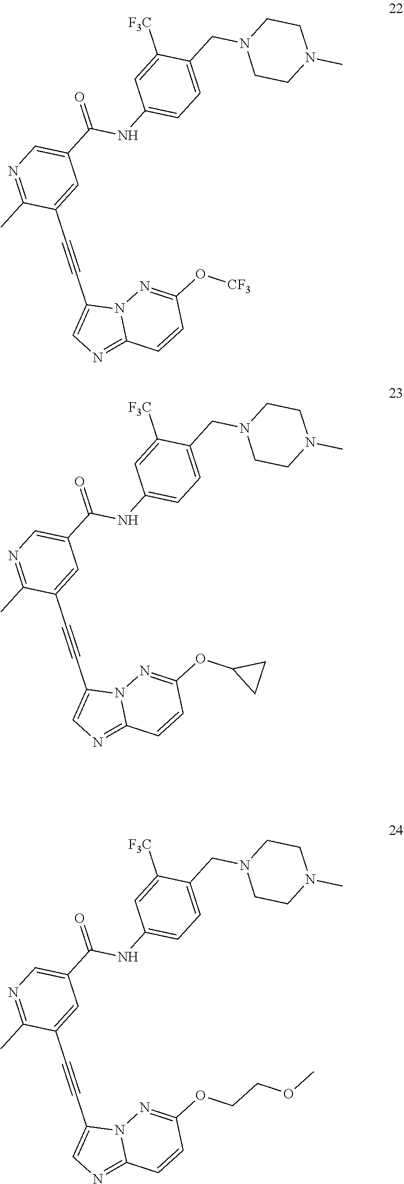

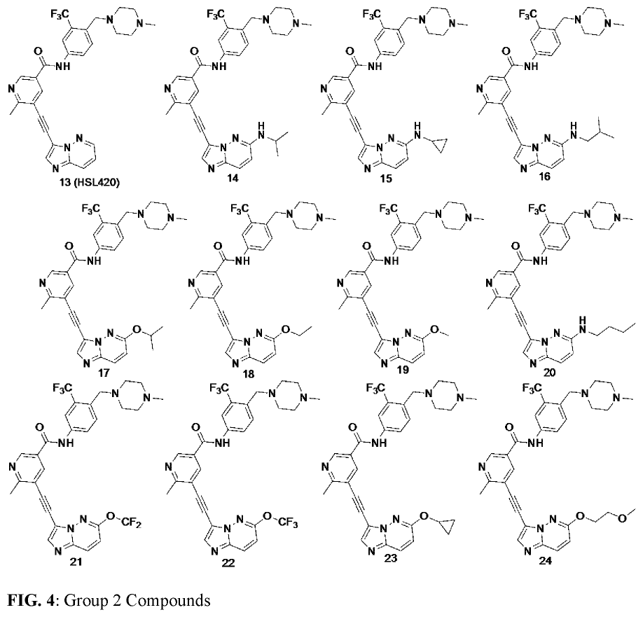

These compounds are hereinafter referred to as Group 2 compounds and are illustrated in FIG. 4.

In some embodiments, the compound of the invention is:

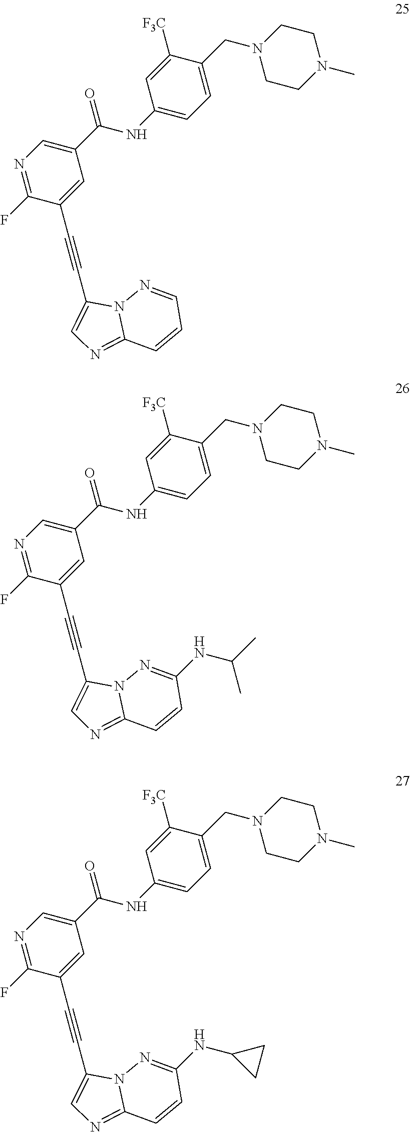 [SMILES: CN1CCN(Cc2ccc(NC(=O)c3cnc(F)c(C#Cc4cnc5cccnn45)c3)cc2C(F)(F)F)CC1 ; CC(C)Nc1ccc2ncc(C#Cc3cc(C(=O)Nc4ccc(CN5CCN(C)CC5)c(C(F)(F)F)c4)cnc3F)n2n1 ; CN1CCN(Cc2ccc(NC(=O)c3cnc(F)c(C#Cc4cnc5ccc(NC6CC6)nn45)c3)cc2C(F)(F)F)CC1]

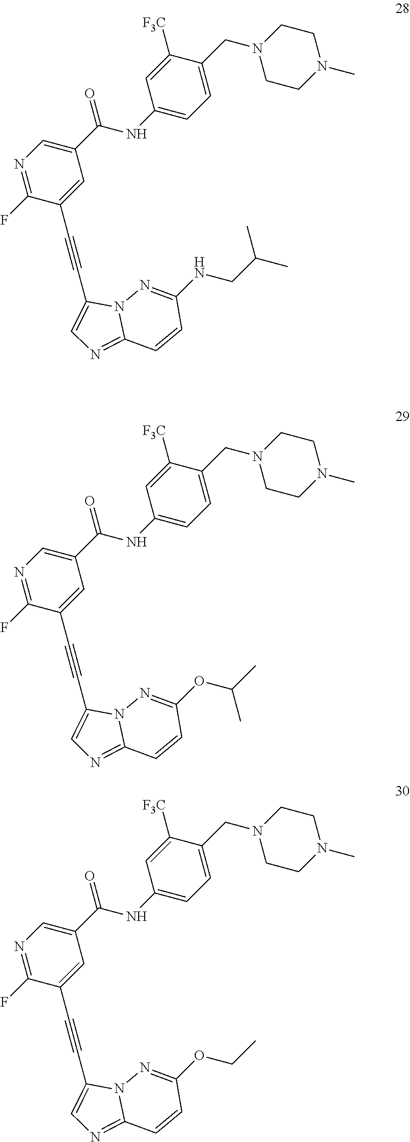 [SMILES: CC(C)CNc1ccc2ncc(C#Cc3cc(C(=O)Nc4ccc(CN5CCN(C)CC5)c(C(F)(F)F)c4)cnc3F)n2n1 ; CC(C)Oc1ccc2ncc(C#Cc3cc(C(=O)Nc4ccc(CN5CCN(C)CC5)c(C(F)(F)F)c4)cnc3F)n2n1 ; CCOc1ccc2ncc(C#Cc3cc(C(=O)Nc4ccc(CN5CCN(C)CC5)c(C(F)(F)F)c4)cnc3F)n2n1]

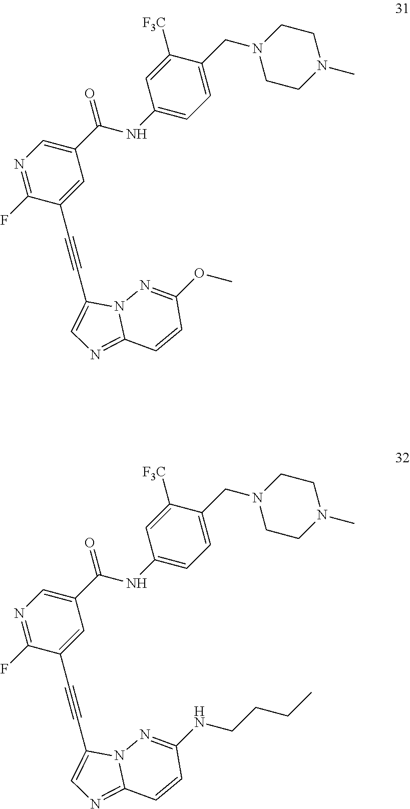 [SMILES: COc1ccc2ncc(C#Cc3cc(C(=O)Nc4ccc(CN5CCN(C)CC5)c(C(F)(F)F)c4)cnc3F)n2n1 ; CCCCNc1ccc2ncc(C#Cc3cc(C(=O)Nc4ccc(CN5CCN(C)CC5)c(C(F)(F)F)c4)cnc3F)n2n1]

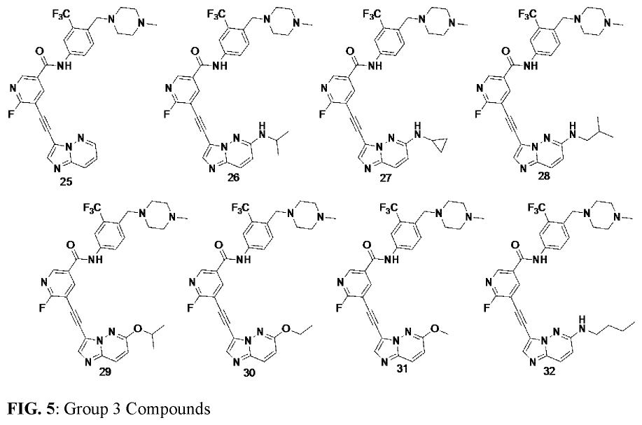

These compounds are hereinafter referred to as Group 3 compounds and are illustrated in FIG. 5.

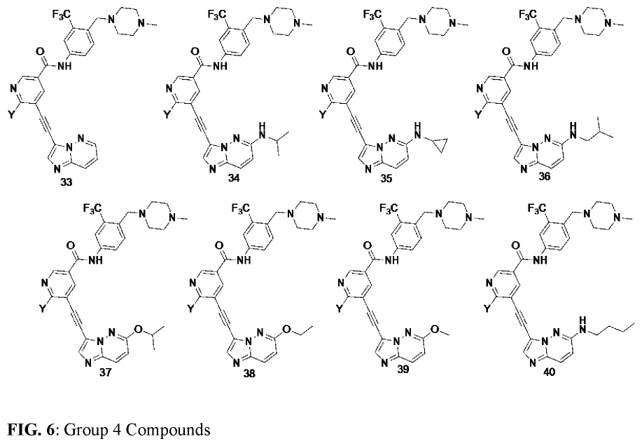

In some embodiments, the compound of the invention is:

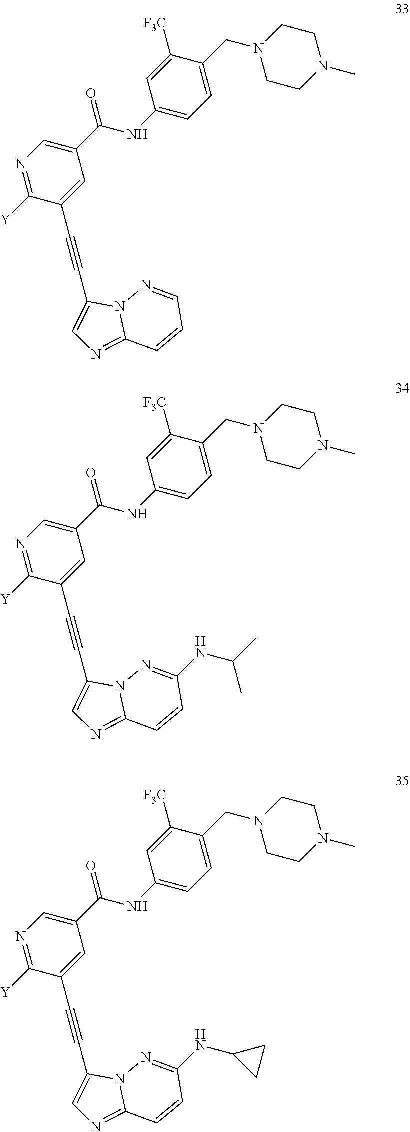

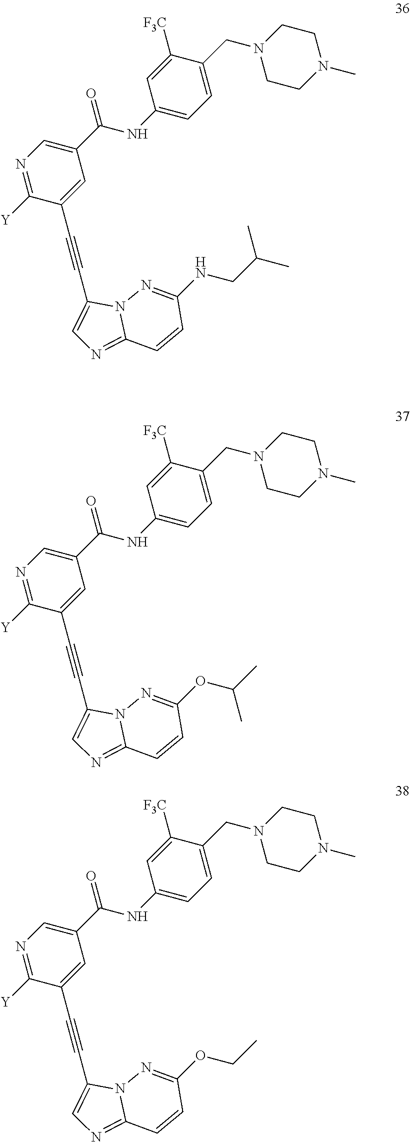

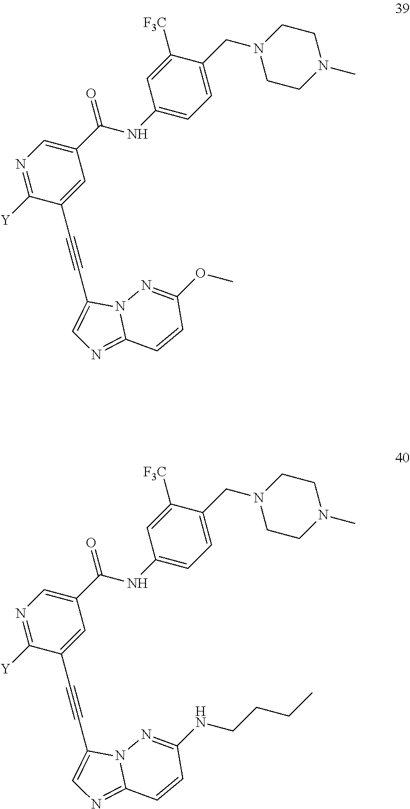

Where Y=amide; O-alkyl such as OMe, OEt, OPr, OBu, OiPr, OCF₂, OCF₃; NH₂, NH-alkyl, such as NHMe, NHEt; N-(alkyl)₂ or N-(heteroalkyl)₂, such as NMe₂, morpholino, piperazine; CN, Cl, Br, iPr, Et, cyclopropyl, butyl etc. These compounds are hereinafter referred to as Group 4 compounds and are illustrated in FIG. 6.

In some embodiments, the compound of the invention is:

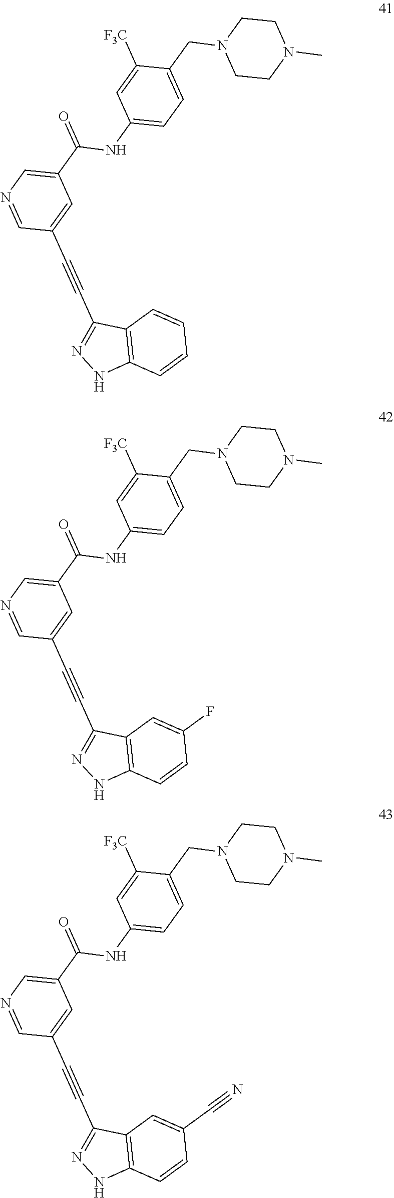 [SMILES: CN1CCN(Cc2ccc(NC(=O)c3cncc(C#Cc4n[nH]c5ccccc45)c3)cc2C(F)(F)F)CC1 ; CN1CCN(Cc2ccc(NC(=O)c3cncc(C#Cc4n[nH]c5ccc(F)cc45)c3)cc2C(F)(F)F)CC1 ; CN1CCN(Cc2ccc(NC(=O)c3cncc(C#Cc4n[nH]c5ccc(C#N)cc45)c3)cc2C(F)(F)F)CC1]

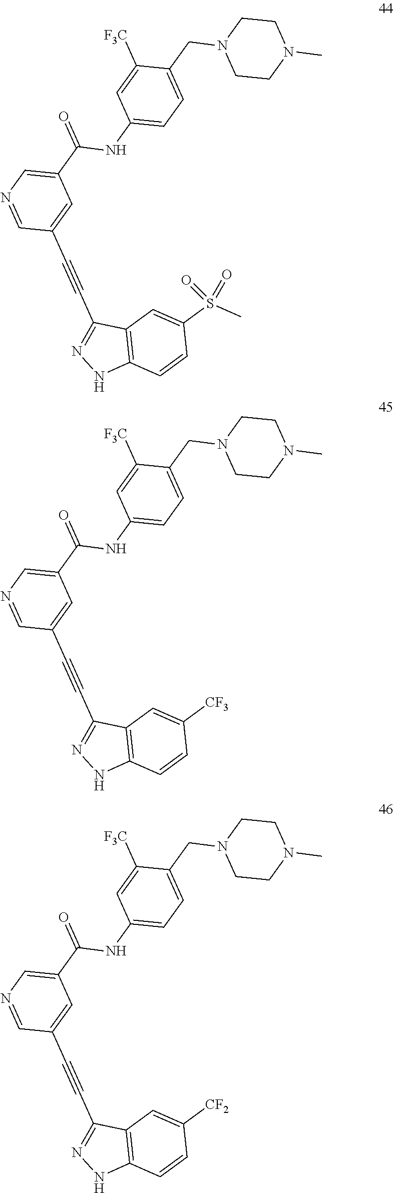

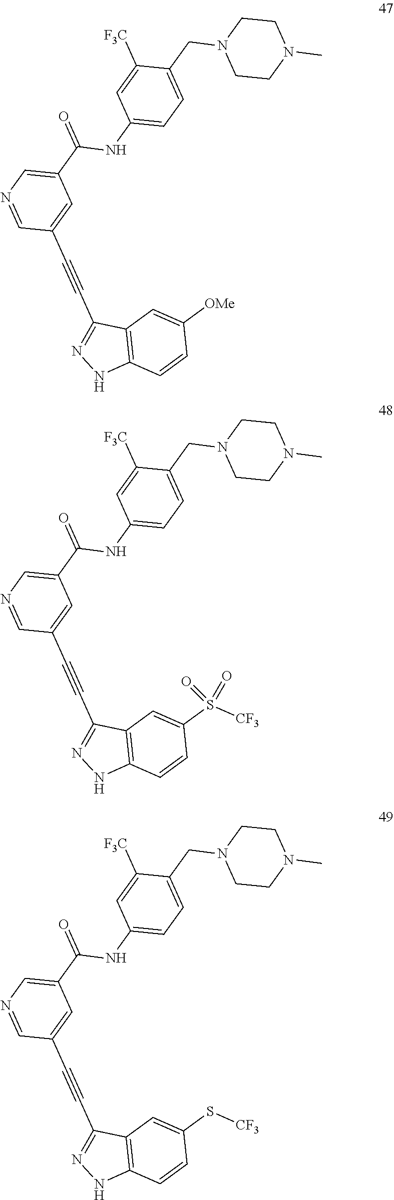 [SMILES: COc1ccc2[nH]nc(C#Cc3cncc(C(=O)Nc4ccc(CN5CCN(C)CC5)c(C(F)(F)F)c4)c3)c2c1 ; CN1CCN(Cc2ccc(NC(=O)c3cncc(C#Cc4n[nH]c5ccc(S(=O)(=O)C(F)(F)F)cc45)c3)cc2C(F)(F)F)CC1 ; CN1CCN(Cc2ccc(NC(=O)c3cncc(C#Cc4n[nH]c5ccc(SC(F)(F)F)cc45)c3)cc2C(F)(F)F)CC1]

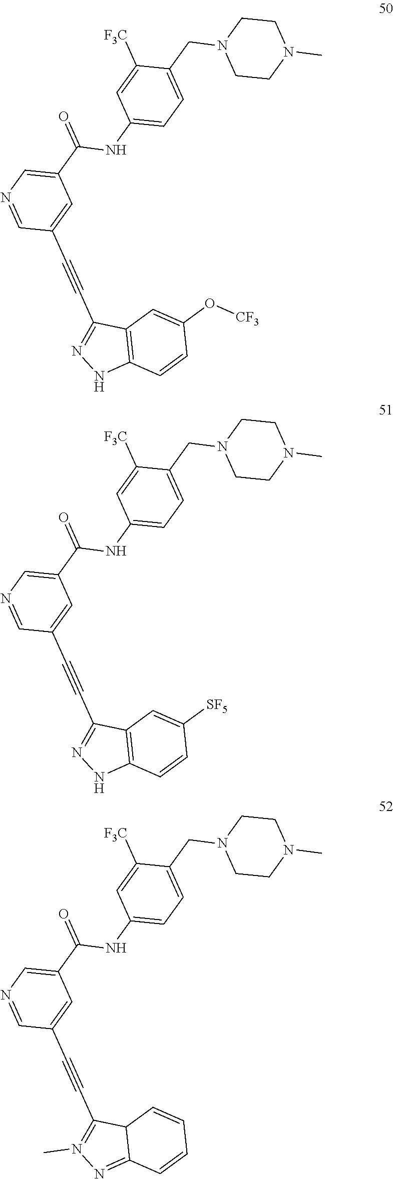

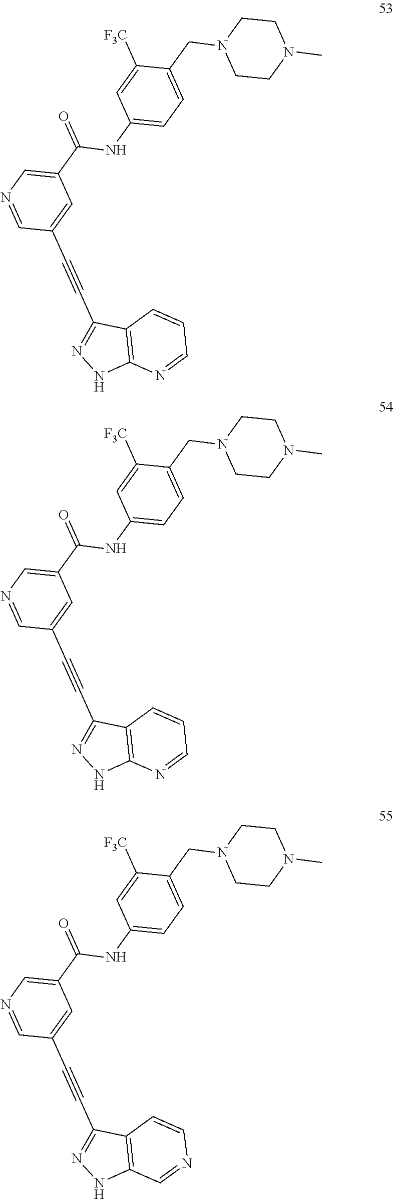 [SMILES: CN1CCN(Cc2ccc(NC(=O)c3cncc(C#Cc4n[nH]c5ncccc45)c3)cc2C(F)(F)F)CC1 ; CN1CCN(Cc2ccc(NC(=O)c3cncc(C#Cc4n[nH]c5ncccc45)c3)cc2C(F)(F)F)CC1 ; CN1CCN(Cc2ccc(NC(=O)c3cncc(C#Cc4n[nH]c5cnccc45)c3)cc2C(F)(F)F)CC1]

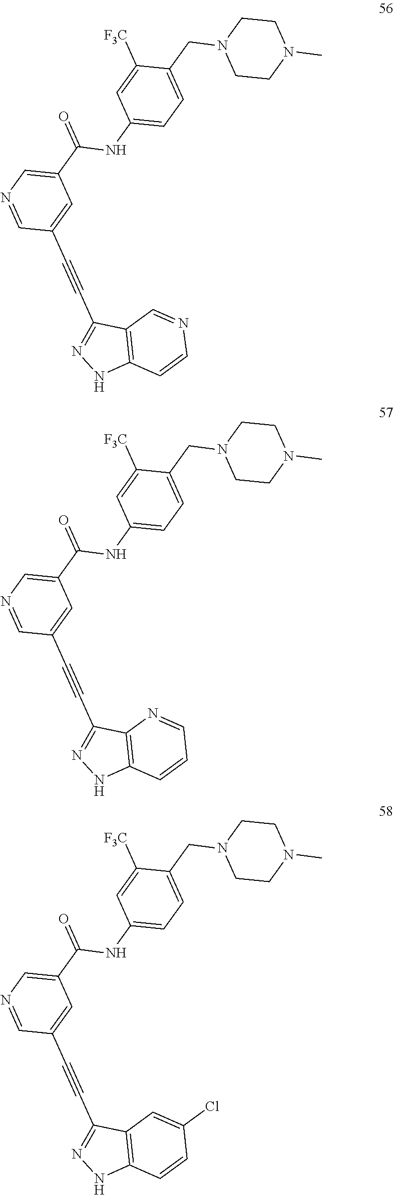 [SMILES: CN1CCN(Cc2ccc(NC(=O)c3cncc(C#Cc4n[nH]c5ccncc45)c3)cc2C(F)(F)F)CC1 ; CN1CCN(Cc2ccc(NC(=O)c3cncc(C#Cc4n[nH]c5cccnc45)c3)cc2C(F)(F)F)CC1 ; CN1CCN(Cc2ccc(NC(=O)c3cncc(C#Cc4n[nH]c5ccc(Cl)cc45)c3)cc2C(F)(F)F)CC1]

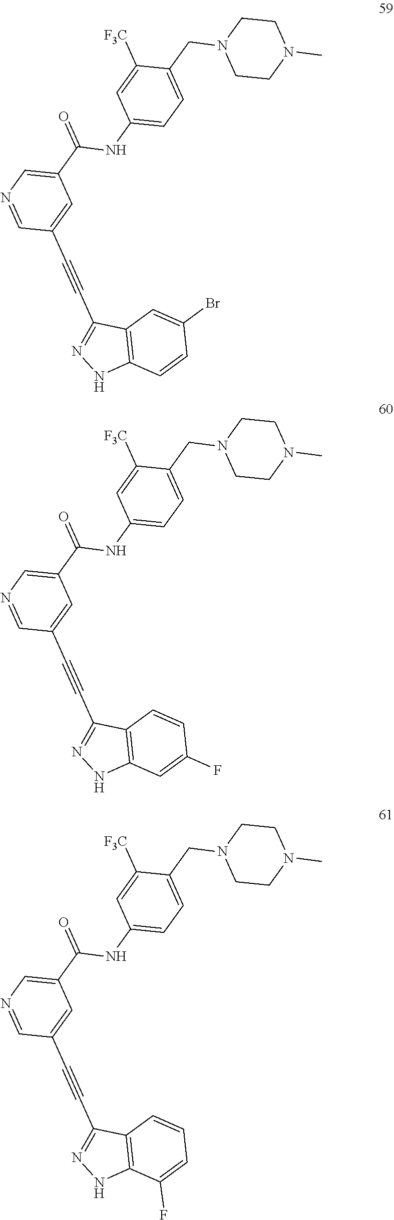 [SMILES: CN1CCN(Cc2ccc(NC(=O)c3cncc(C#Cc4n[nH]c5ccc(Br)cc45)c3)cc2C(F)(F)F)CC1 ; CN1CCN(Cc2ccc(NC(=O)c3cncc(C#Cc4n[nH]c5cc(F)ccc45)c3)cc2C(F)(F)F)CC1 ; CN1CCN(Cc2ccc(NC(=O)c3cncc(C#Cc4n[nH]c5c(F)cccc45)c3)cc2C(F)(F)F)CC1]

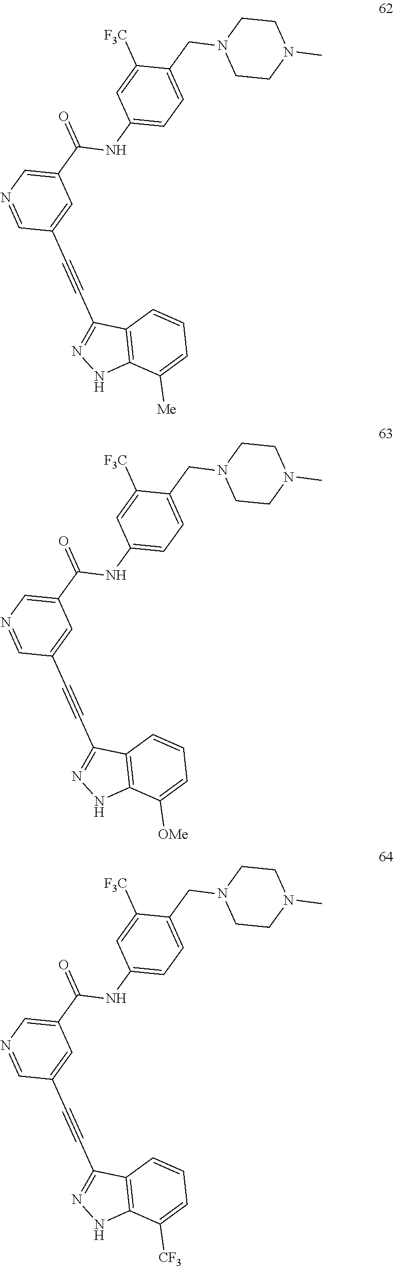 [SMILES: Cc1cccc2c(C#Cc3cncc(C(=O)Nc4ccc(CN5CCN(C)CC5)c(C(F)(F)F)c4)c3)n[nH]c12 ; COc1cccc2c(C#Cc3cncc(C(=O)Nc4ccc(CN5CCN(C)CC5)c(C(F)(F)F)c4)c3)n[nH]c12 ; CN1CCN(Cc2ccc(NC(=O)c3cncc(C#Cc4n[nH]c5c(C(F)(F)F)cccc45)c3)cc2C(F)(F)F)CC1]

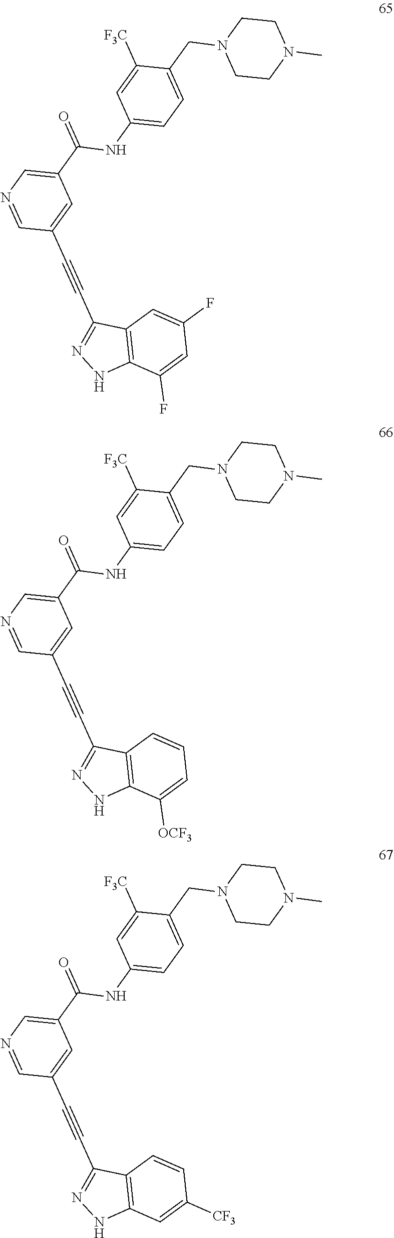 [SMILES: CN1CCN(Cc2ccc(NC(=O)c3cncc(C#Cc4n[nH]c5c(F)cc(F)cc45)c3)cc2C(F)(F)F)CC1 ; CN1CCN(Cc2ccc(NC(=O)c3cncc(C#Cc4n[nH]c5c(OC(F)(F)F)cccc45)c3)cc2C(F)(F)F)CC1 ; CN1CCN(Cc2ccc(NC(=O)c3cncc(C#Cc4n[nH]c5cc(C(F)(F)F)ccc45)c3)cc2C(F)(F)F)CC1]

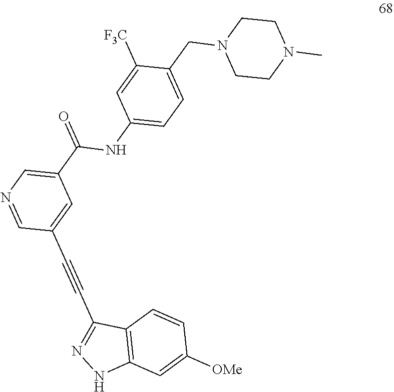 [SMILES: COc1ccc2c(C#Cc3cncc(C(=O)Nc4ccc(CN5CCN(C)CC5)c(C(F)(F)F)c4)c3)n[nH]c2c1]

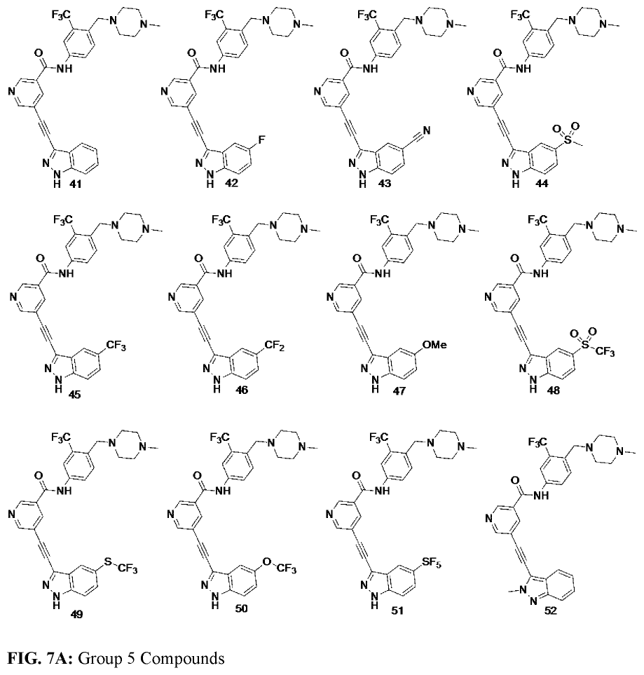

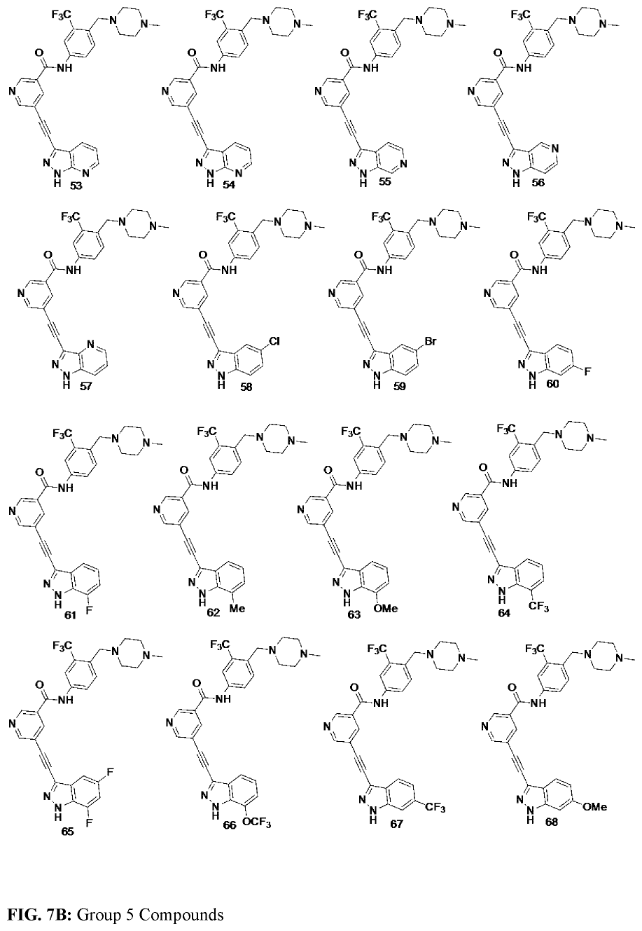

These compounds are hereinafter referred to as Group 5 compounds and are illustrated in FIGS. 7A and 7B.

In some embodiments, the compound of the invention is:

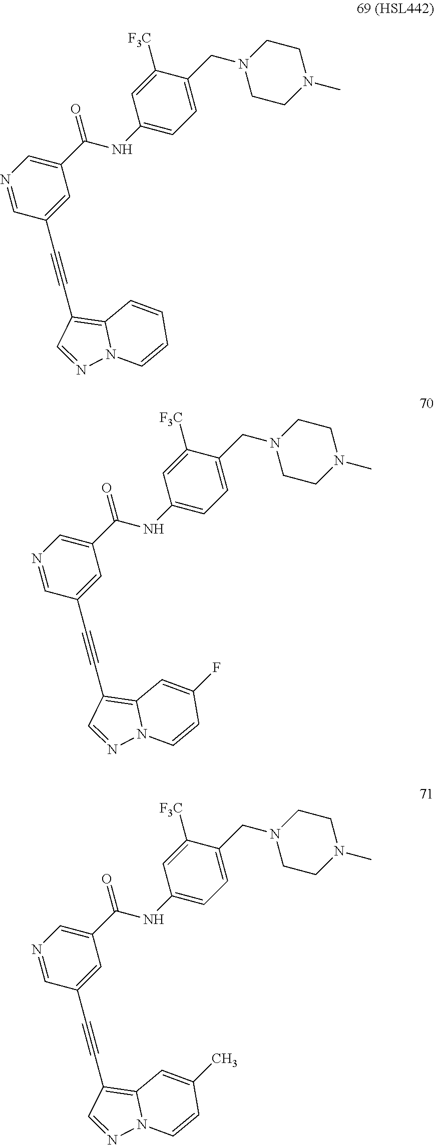 [SMILES: CN1CCN(Cc2ccc(NC(=O)c3cncc(C#Cc4cnn5ccccc45)c3)cc2C(F)(F)F)CC1 ; CN1CCN(Cc2ccc(NC(=O)c3cncc(C#Cc4cnn5ccc(F)cc45)c3)cc2C(F)(F)F)CC1 ; Cc1ccn2ncc(C#Cc3cncc(C(=O)Nc4ccc(CN5CCN(C)CC5)c(C(F)(F)F)c4)c3)c2c1]

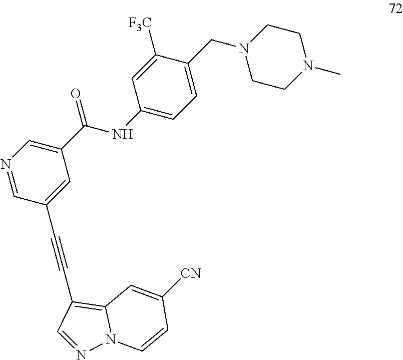 [SMILES: CN1CCN(Cc2ccc(NC(=O)c3cncc(C#Cc4cnn5ccc(C#N)cc45)c3)cc2C(F)(F)F)CC1]

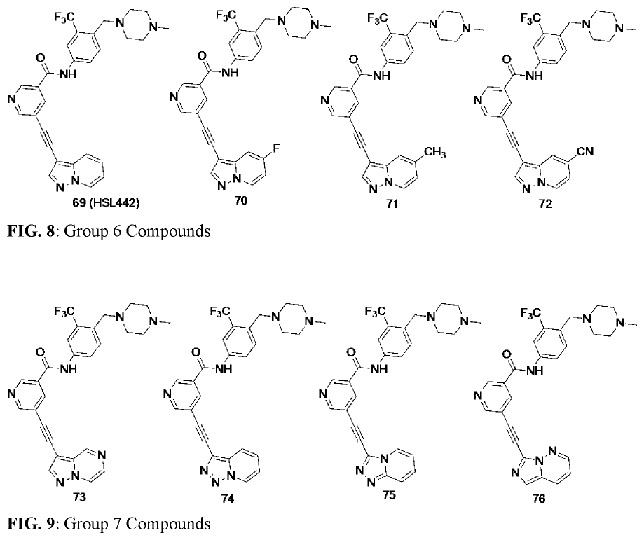

These compounds are hereinafter referred to as Group 6 compounds and are illustrated in FIG. 8.

In some embodiments, the compound of the invention is:

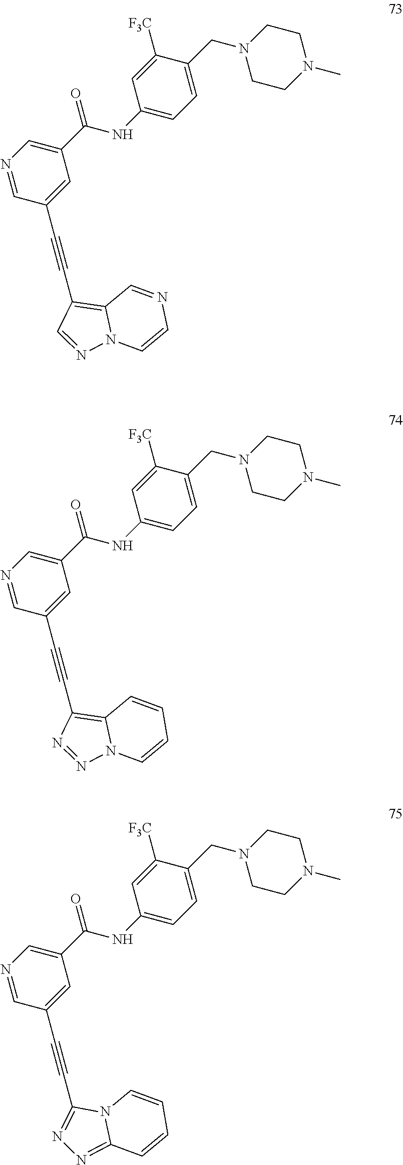 [SMILES: CN1CCN(Cc2ccc(NC(=O)c3cncc(C#Cc4cnn5ccncc45)c3)cc2C(F)(F)F)CC1 ; CN1CCN(Cc2ccc(NC(=O)c3cncc(C#Cc4nnn5ccccc45)c3)cc2C(F)(F)F)CC1 ; CN1CCN(Cc2ccc(NC(=O)c3cncc(C#Cc4nnc5ccccn45)c3)cc2C(F)(F)F)CC1]

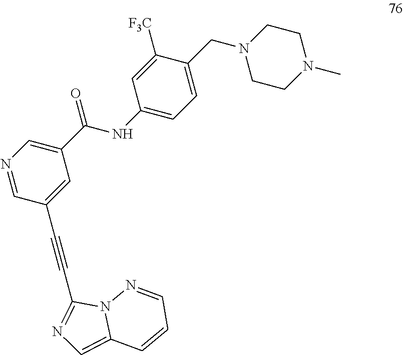 [SMILES: CN1CCN(Cc2ccc(NC(=O)c3cncc(C#Cc4ncc5cccnn45)c3)cc2C(F)(F)F)CC1]

These compounds are hereinafter referred to as Group 7 compounds and are illustrated in FIG. 9.

In some embodiments, the compound of the invention is:

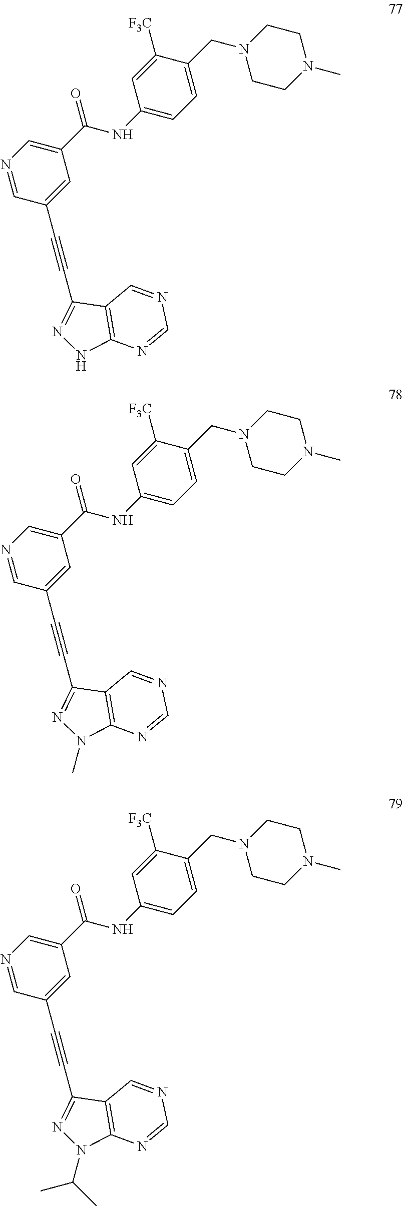 [SMILES: CN1CCN(Cc2ccc(NC(=O)c3cncc(C#Cc4n[nH]c5ncncc45)c3)cc2C(F)(F)F)CC1 ; CN1CCN(Cc2ccc(NC(=O)c3cncc(C#Cc4nn(C)c5ncncc45)c3)cc2C(F)(F)F)CC1 ; CC(C)n1nc(C#Cc2cncc(C(=O)Nc3ccc(CN4CCN(C)CC4)c(C(F)(F)F)c3)c2)c2cncnc21]

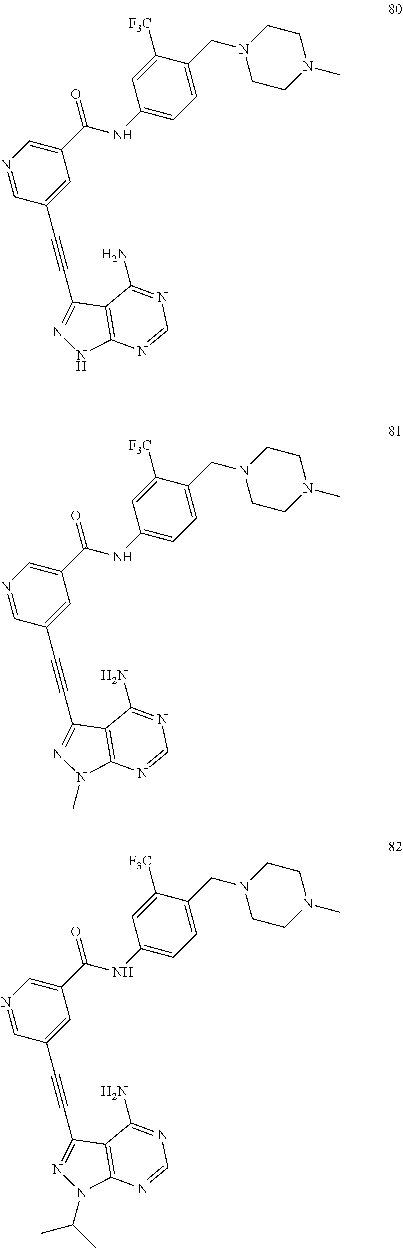 [SMILES: CN1CCN(Cc2ccc(NC(=O)c3cncc(C#Cc4n[nH]c5ncnc(N)c45)c3)cc2C(F)(F)F)CC1 ; CN1CCN(Cc2ccc(NC(=O)c3cncc(C#Cc4nn(C)c5ncnc(N)c45)c3)cc2C(F)(F)F)CC1 ; CC(C)n1nc(C#Cc2cncc(C(=O)Nc3ccc(CN4CCN(C)CC4)c(C(F)(F)F)c3)c2)c2c(N)ncnc21]

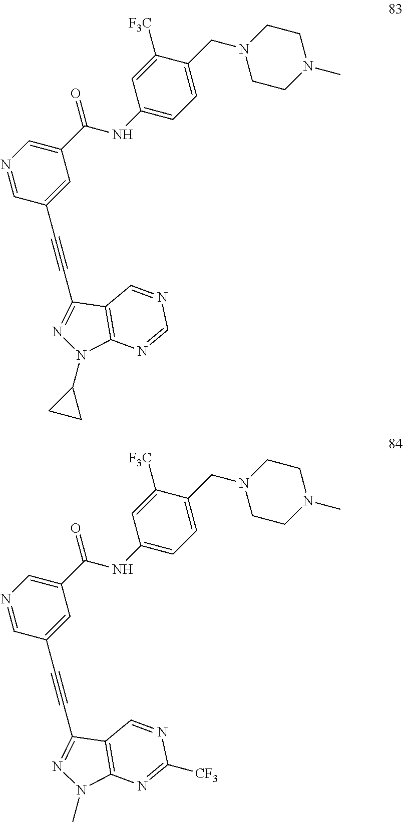 [SMILES: CN1CCN(Cc2ccc(NC(=O)c3cncc(C#Cc4nn(C5CC5)c5ncncc45)c3)cc2C(F)(F)F)CC1 ; CN1CCN(Cc2ccc(NC(=O)c3cncc(C#Cc4nn(C)c5nc(C(F)(F)F)ncc45)c3)cc2C(F)(F)F)CC1]

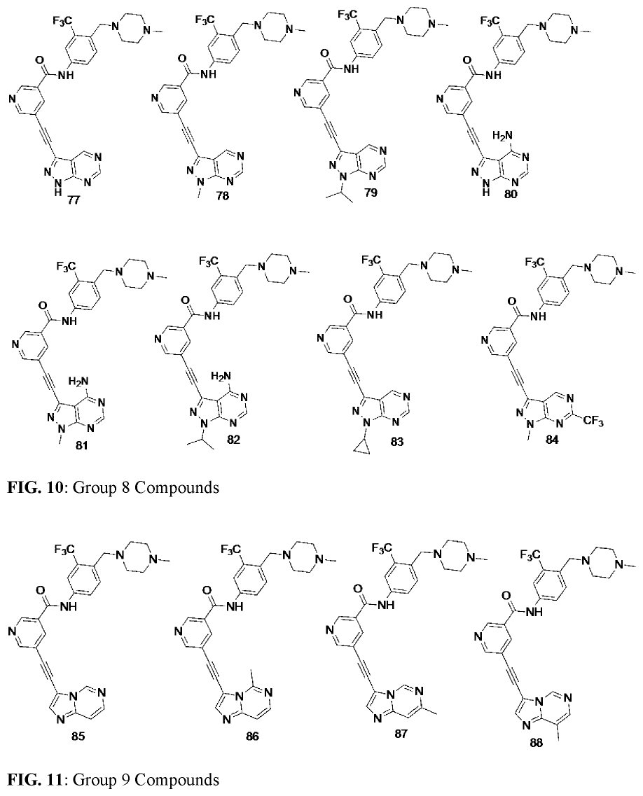

These compounds are hereinafter referred to as Group 8 compounds and are illustrated in FIG. 10.

In some embodiments, the compound of the invention is:

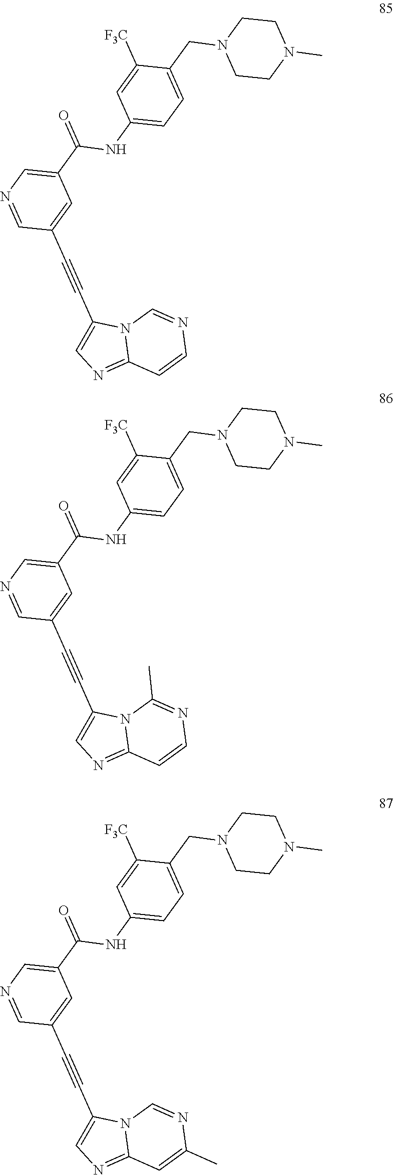 [SMILES: CN1CCN(Cc2ccc(NC(=O)c3cncc(C#Cc4cnc5ccncn45)c3)cc2C(F)(F)F)CC1 ; Cc1nccc2ncc(C#Cc3cncc(C(=O)Nc4ccc(CN5CCN(C)CC5)c(C(F)(F)F)c4)c3)n12 ; Cc1cc2ncc(C#Cc3cncc(C(=O)Nc4ccc(CN5CCN(C)CC5)c(C(F)(F)F)c4)c3)n2cn1]

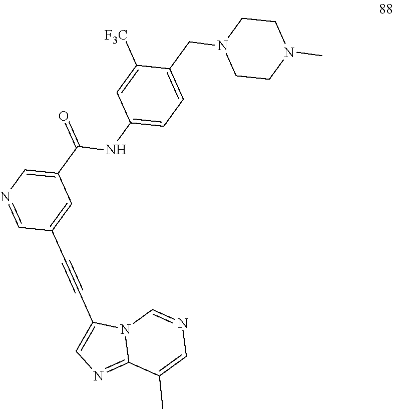 [SMILES: Cc1cncn2c(C#Cc3cncc(C(=O)Nc4ccc(CN5CCN(C)CC5)c(C(F)(F)F)c4)c3)cnc12]

These compounds are hereinafter referred to as Group 9 compounds and are illustrated in FIG. 11.

In some embodiments, the compound of the invention is:

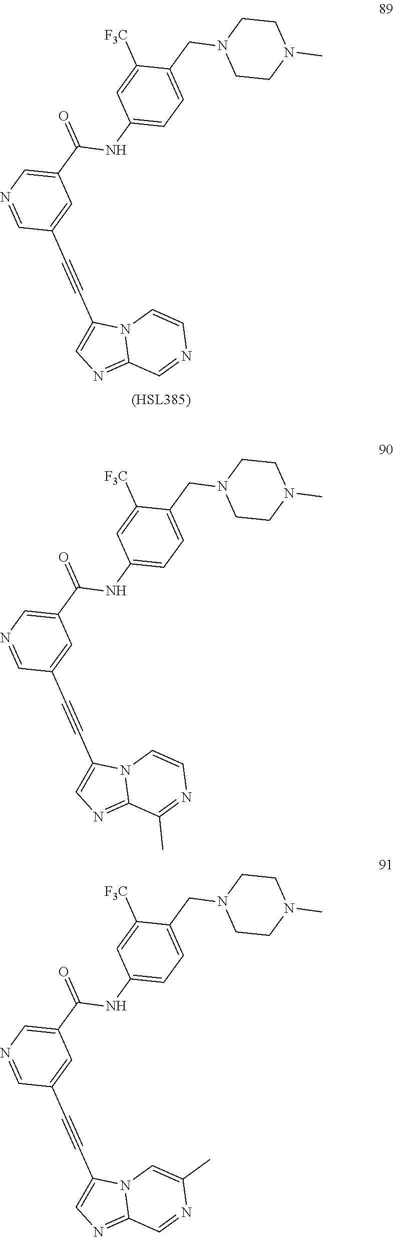 [SMILES: CN1CCN(Cc2ccc(NC(=O)c3cncc(C#Cc4cnc5cnccn45)c3)cc2C(F)(F)F)CC1 ; Cc1nccn2c(C#Cc3cncc(C(=O)Nc4ccc(CN5CCN(C)CC5)c(C(F)(F)F)c4)c3)cnc12 ; Cc1cn2c(C#Cc3cncc(C(=O)Nc4ccc(CN5CCN(C)CC5)c(C(F)(F)F)c4)c3)cnc2cn1]

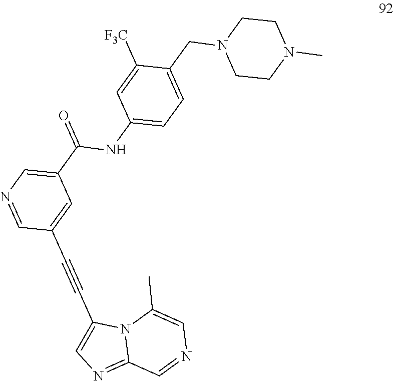 [SMILES: Cc1cncc2ncc(C#Cc3cncc(C(=O)Nc4ccc(CN5CCN(C)CC5)c(C(F)(F)F)c4)c3)n12]

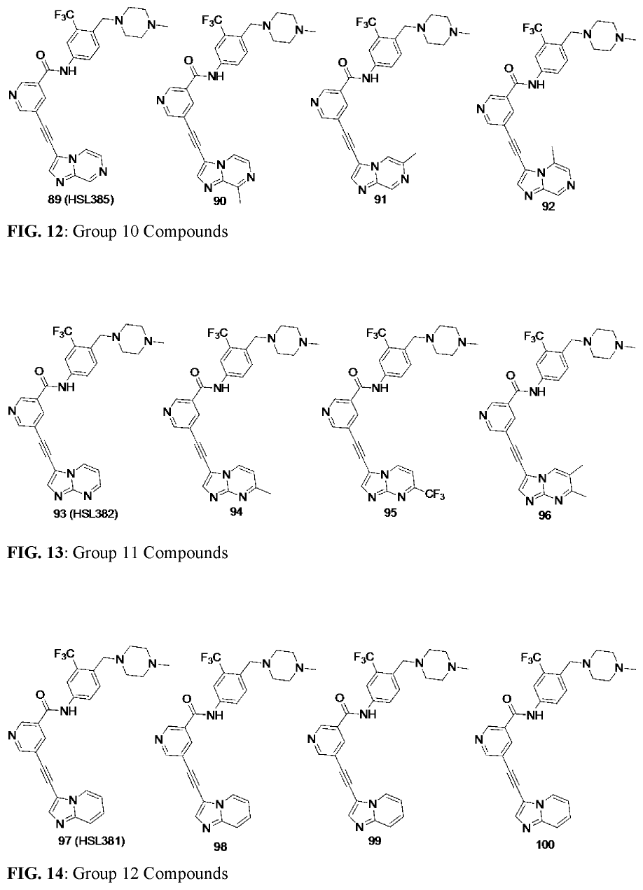

These compounds are hereinafter referred to as Group 10 compounds and are illustrated in FIG. 12.

In some embodiments, the compound of the invention is:

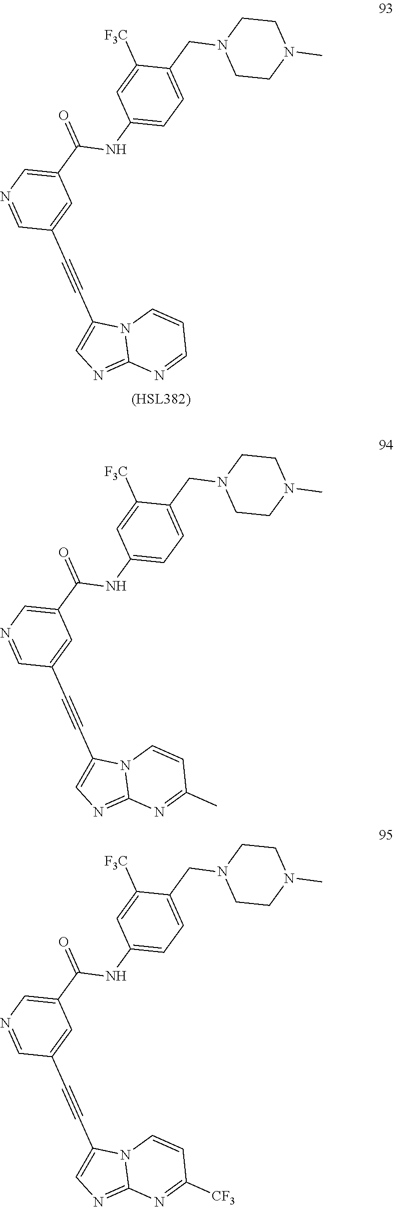

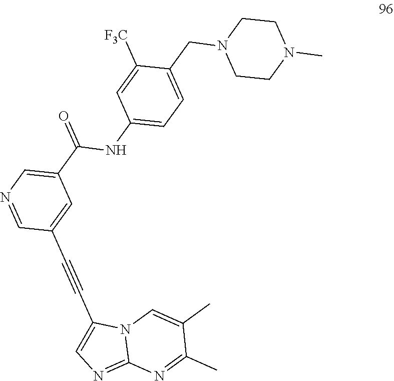 [SMILES: Cc1cn2c(C#Cc3cncc(C(=O)Nc4ccc(CN5CCN(C)CC5)c(C(F)(F)F)c4)c3)cnc2nc1C]

These compounds are hereinafter referred to as Group 11 compounds and are illustrated in FIG. 13.

In some embodiments, the compound of the invention is:

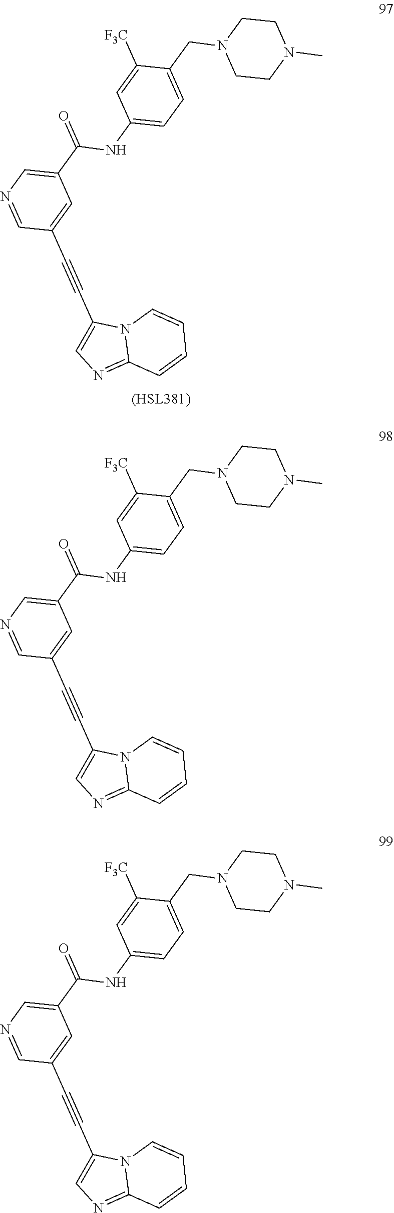 [SMILES: CN1CCN(Cc2ccc(NC(=O)c3cncc(C#Cc4cnc5ccccn45)c3)cc2C(F)(F)F)CC1 ; CN1CCN(Cc2ccc(NC(=O)c3cncc(C#Cc4cnc5ccccn45)c3)cc2C(F)(F)F)CC1 ; CN1CCN(Cc2ccc(NC(=O)c3cncc(C#Cc4cnc5ccccn45)c3)cc2C(F)(F)F)CC1]

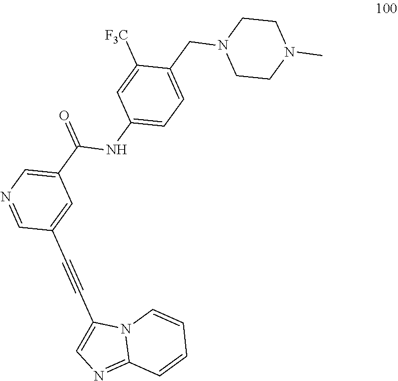 [SMILES: CN1CCN(Cc2ccc(NC(=O)c3cncc(C#Cc4cnc5ccccn45)c3)cc2C(F)(F)F)CC1]

These compounds are hereinafter referred to as Group 12 compounds and are illustrated in FIG. 14.

In some embodiments, the compound of the invention is:

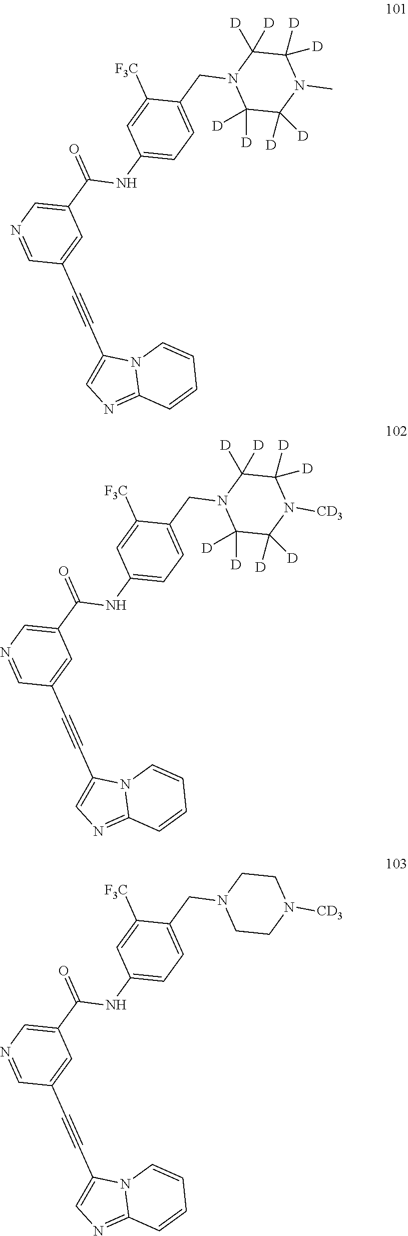 [SMILES: [2H]C1([2H])N(C)C([2H])([2H])C([2H])([2H])N(Cc2ccc(NC(=O)c3cncc(C#Cc4cnc5ccccn45)c3)cc2C(F)(F)F)C1([2H])[2H] ; [2H]C([2H])([2H])N1C([2H])([2H])C([2H])([2H])N(Cc2ccc(NC(=O)c3cncc(C#Cc4cnc5ccccn45)c3)cc2C(F)(F)F)C([2H])([2H])C1([2H])[2H] ; [2H]C([2H])([2H])N1CCN(Cc2ccc(NC(=O)c3cncc(C#Cc4cnc5ccccn45)c3)cc2C(F)(F)F)CC1]

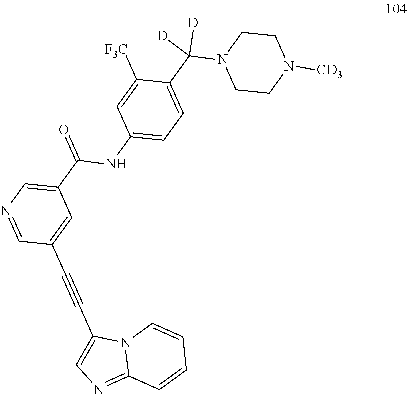 [SMILES: [2H]C([2H])([2H])N1CCN(C([2H])([2H])c2ccc(NC(=O)c3cncc(C#Cc4cnc5ccccn45)c3)cc2C(F)(F)F)CC1]

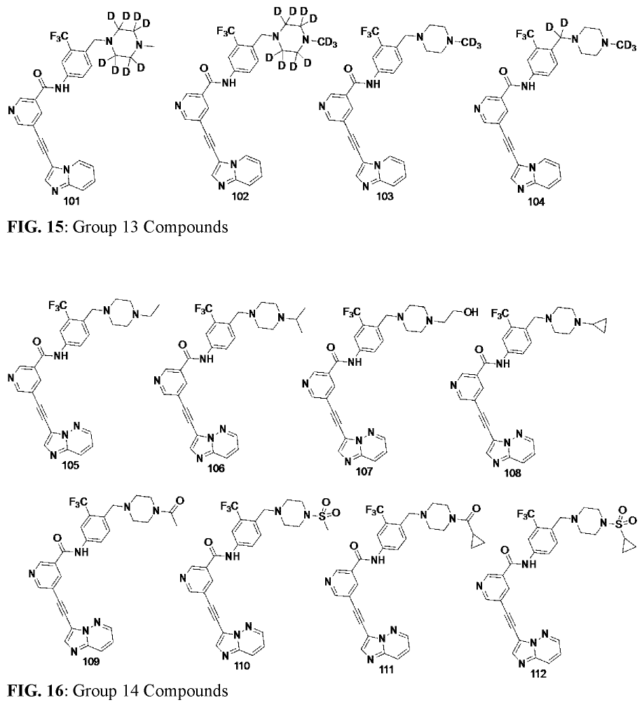

These compounds are hereinafter referred to as Group 13 compounds and are illustrated in FIG. 15.

In some embodiments, the compound of the invention is:

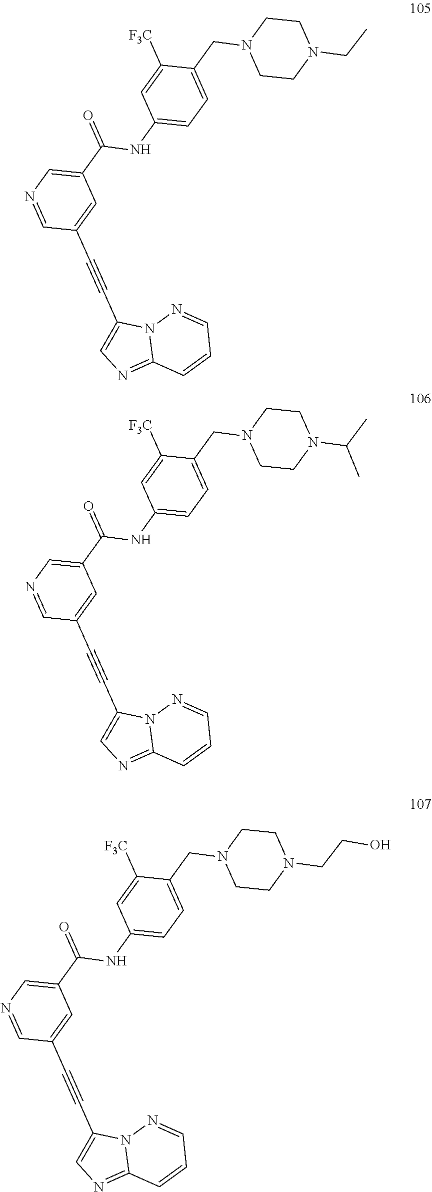 [SMILES: CCN1CCN(Cc2ccc(NC(=O)c3cncc(C#Cc4cnc5cccnn45)c3)cc2C(F)(F)F)CC1 ; CC(C)N1CCN(Cc2ccc(NC(=O)c3cncc(C#Cc4cnc5cccnn45)c3)cc2C(F)(F)F)CC1 ; O=C(Nc1ccc(CN2CCN(CCO)CC2)c(C(F)(F)F)c1)c1cncc(C#Cc2cnc3cccnn23)c1]

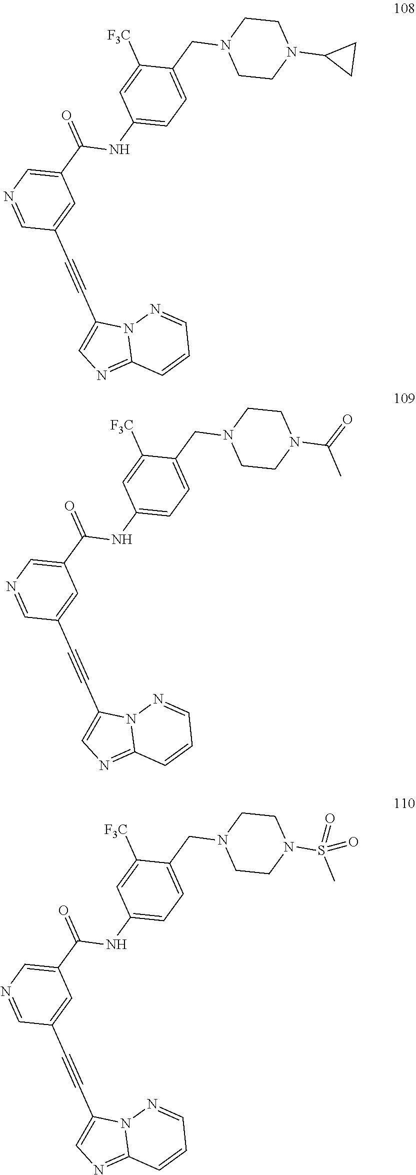 [SMILES: O=C(Nc1ccc(CN2CCN(C3CC3)CC2)c(C(F)(F)F)c1)c1cncc(C#Cc2cnc3cccnn23)c1 ; CC(=O)N1CCN(Cc2ccc(NC(=O)c3cncc(C#Cc4cnc5cccnn45)c3)cc2C(F)(F)F)CC1 ; CS(=O)(=O)N1CCN(Cc2ccc(NC(=O)c3cncc(C#Cc4cnc5cccnn45)c3)cc2C(F)(F)F)CC1]

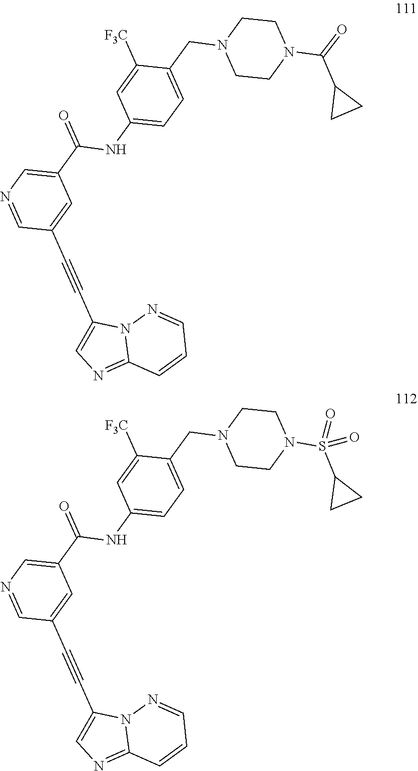 [SMILES: O=C(Nc1ccc(CN2CCN(C(=O)C3CC3)CC2)c(C(F)(F)F)c1)c1cncc(C#Cc2cnc3cccnn23)c1 ; O=C(Nc1ccc(CN2CCN(S(=O)(=O)C3CC3)CC2)c(C(F)(F)F)c1)c1cncc(C#Cc2cnc3cccnn23)c1]

These compounds are hereinafter referred to as Group 14 compounds and are illustrated in FIG. 16.

In some embodiments, the compound of the invention is:

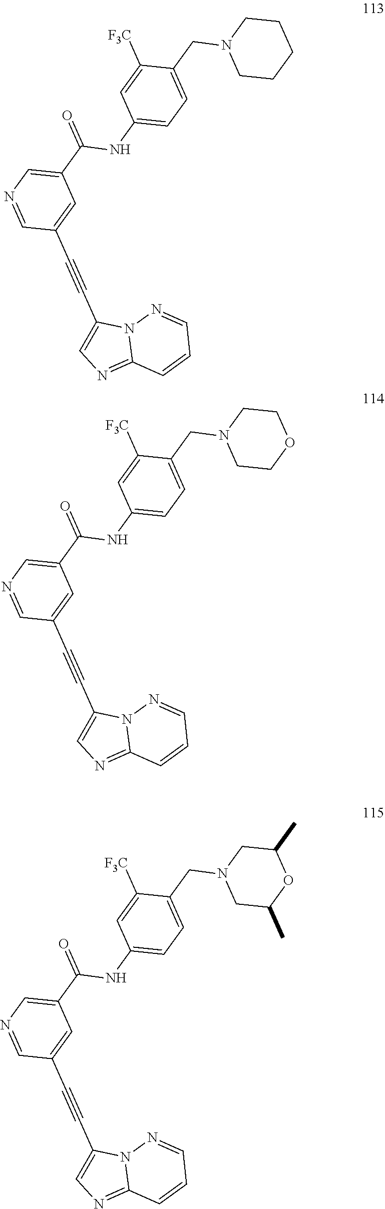 [SMILES: O=C(Nc1ccc(CN2CCCCC2)c(C(F)(F)F)c1)c1cncc(C#Cc2cnc3cccnn23)c1 ; O=C(Nc1ccc(CN2CCOCC2)c(C(F)(F)F)c1)c1cncc(C#Cc2cnc3cccnn23)c1 ; C[C@@H]1CN(Cc2ccc(NC(=O)c3cncc(C#Cc4cnc5cccnn45)c3)cc2C(F)(F)F)C[C@H](C)O1]

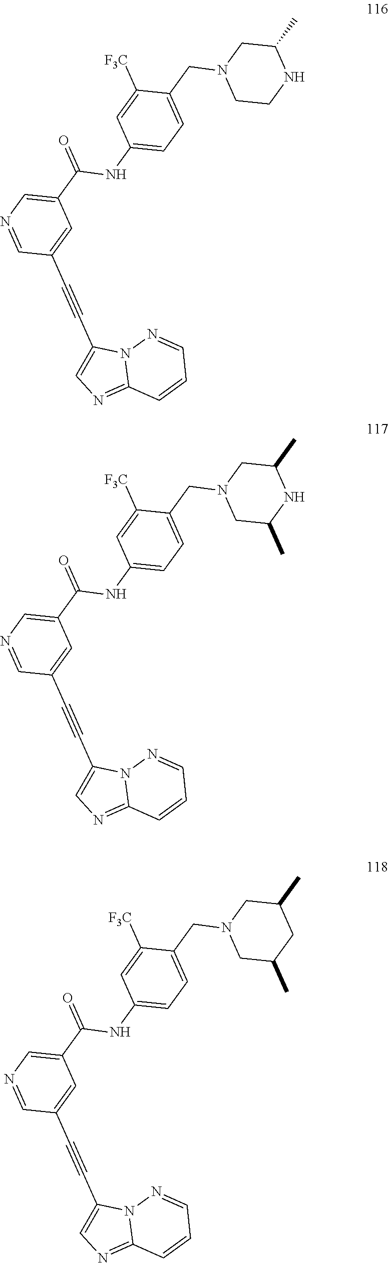 [SMILES: C[C@H]1CN(Cc2ccc(NC(=O)c3cncc(C#Cc4cnc5cccnn45)c3)cc2C(F)(F)F)CCN1 ; C[C@@H]1CN(Cc2ccc(NC(=O)c3cncc(C#Cc4cnc5cccnn45)c3)cc2C(F)(F)F)C[C@H](C)N1 ; C[C@@H]1C[C@H](C)CN(Cc2ccc(NC(=O)c3cncc(C#Cc4cnc5cccnn45)c3)cc2C(F)(F)F)C1]

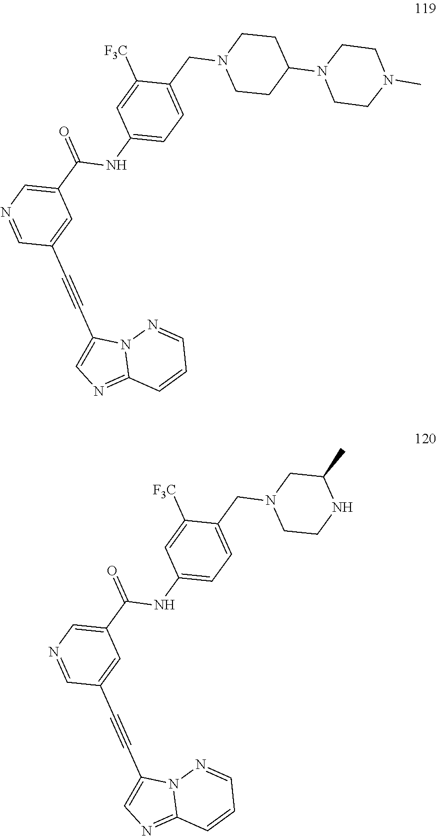 [SMILES: CN1CCN(C2CCN(Cc3ccc(NC(=O)c4cncc(C#Cc5cnc6cccnn56)c4)cc3C(F)(F)F)CC2)CC1 ; C[C@@H]1CN(Cc2ccc(NC(=O)c3cncc(C#Cc4cnc5cccnn45)c3)cc2C(F)(F)F)CCN1]

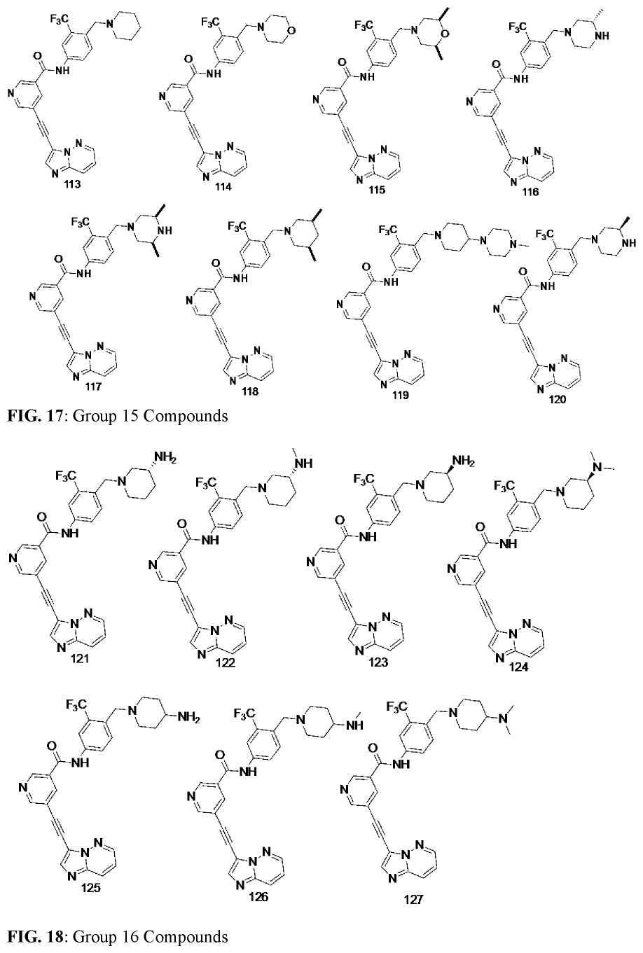

These compounds are hereinafter referred to as Group 15 compounds and are illustrated in FIG. 17.

In some embodiments, the compound of the invention is:

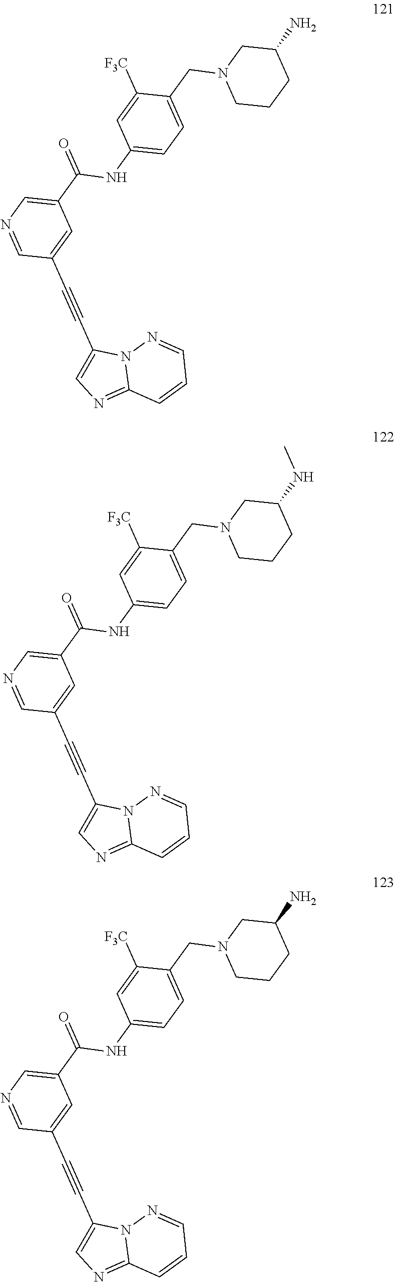 [SMILES: N[C@@H]1CCCN(Cc2ccc(NC(=O)c3cncc(C#Cc4cnc5cccnn45)c3)cc2C(F)(F)F)C1 ; CN[C@@H]1CCCN(Cc2ccc(NC(=O)c3cncc(C#Cc4cnc5cccnn45)c3)cc2C(F)(F)F)C1 ; N[C@H]1CCCN(Cc2ccc(NC(=O)c3cncc(C#Cc4cnc5cccnn45)c3)cc2C(F)(F)F)C1]

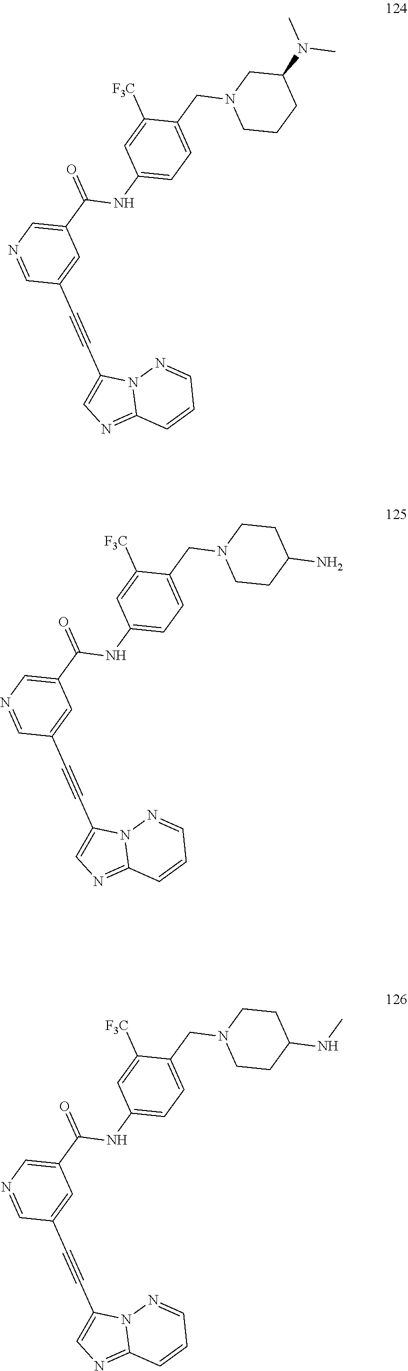 [SMILES: CN(C)[C@H]1CCCN(Cc2ccc(NC(=O)c3cncc(C#Cc4cnc5cccnn45)c3)cc2C(F)(F)F)C1 ; NC1CCN(Cc2ccc(NC(=O)c3cncc(C#Cc4cnc5cccnn45)c3)cc2C(F)(F)F)CC1 ; CNC1CCN(Cc2ccc(NC(=O)c3cncc(C#Cc4cnc5cccnn45)c3)cc2C(F)(F)F)CC1]

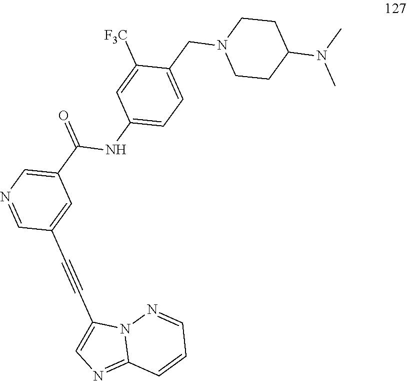 [SMILES: CN(C)C1CCN(Cc2ccc(NC(=O)c3cncc(C#Cc4cnc5cccnn45)c3)cc2C(F)(F)F)CC1]

These compounds are hereinafter referred to as Group 16 compounds and are illustrated in FIG. 18.

In some embodiments, the compound of the invention is:

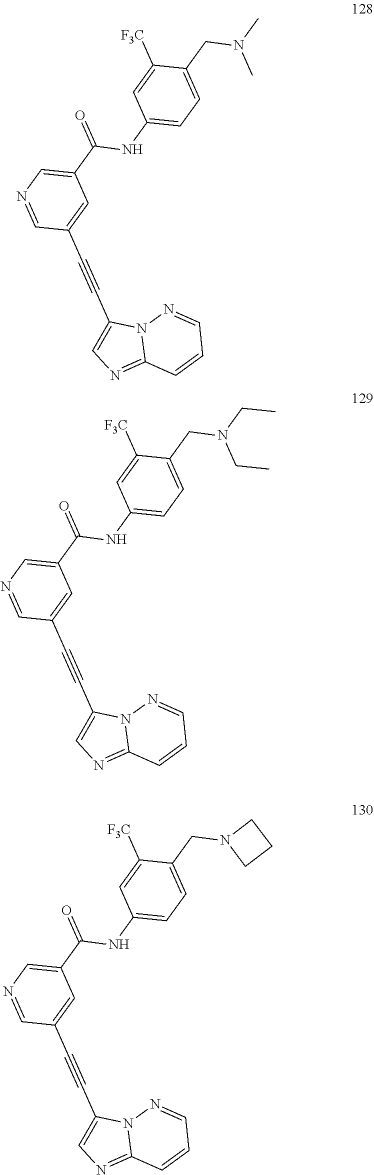 [SMILES: CN(C)Cc1ccc(NC(=O)c2cncc(C#Cc3cnc4cccnn34)c2)cc1C(F)(F)F ; CCN(CC)Cc1ccc(NC(=O)c2cncc(C#Cc3cnc4cccnn34)c2)cc1C(F)(F)F ; O=C(Nc1ccc(CN2CCC2)c(C(F)(F)F)c1)c1cncc(C#Cc2cnc3cccnn23)c1]

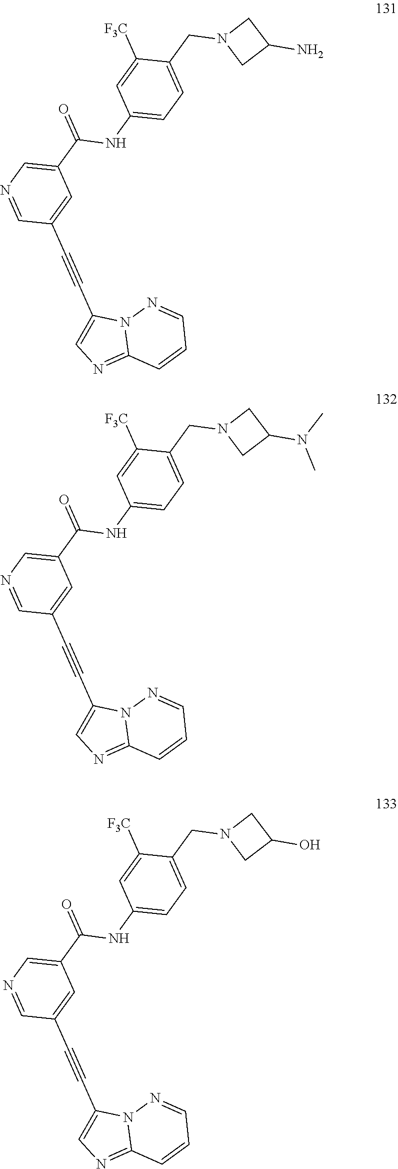 [SMILES: NC1CN(Cc2ccc(NC(=O)c3cncc(C#Cc4cnc5cccnn45)c3)cc2C(F)(F)F)C1 ; CN(C)C1CN(Cc2ccc(NC(=O)c3cncc(C#Cc4cnc5cccnn45)c3)cc2C(F)(F)F)C1 ; O=C(Nc1ccc(CN2CC(O)C2)c(C(F)(F)F)c1)c1cncc(C#Cc2cnc3cccnn23)c1]

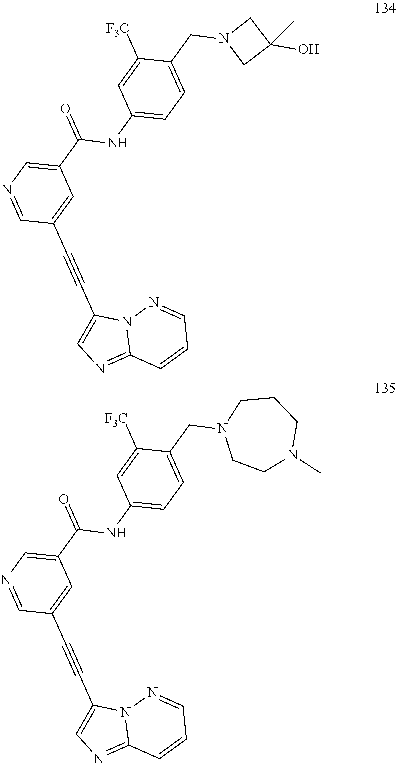 [SMILES: CC1(O)CN(Cc2ccc(NC(=O)c3cncc(C#Cc4cnc5cccnn45)c3)cc2C(F)(F)F)C1 ; CN1CCCN(Cc2ccc(NC(=O)c3cncc(C#Cc4cnc5cccnn45)c3)cc2C(F)(F)F)CC1]

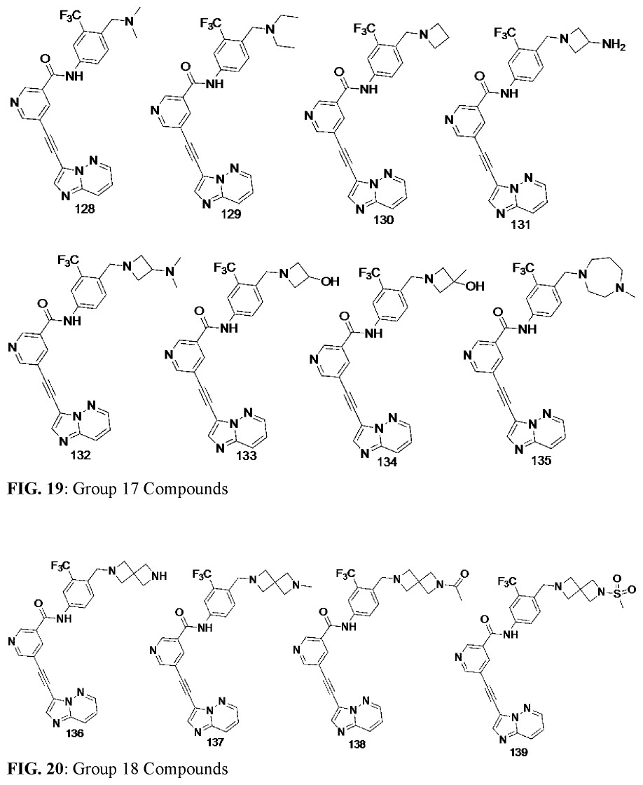

These compounds are hereinafter referred to as Group 17 compounds and are illustrated in FIG. 19.

In some embodiments, the compound of the invention is:

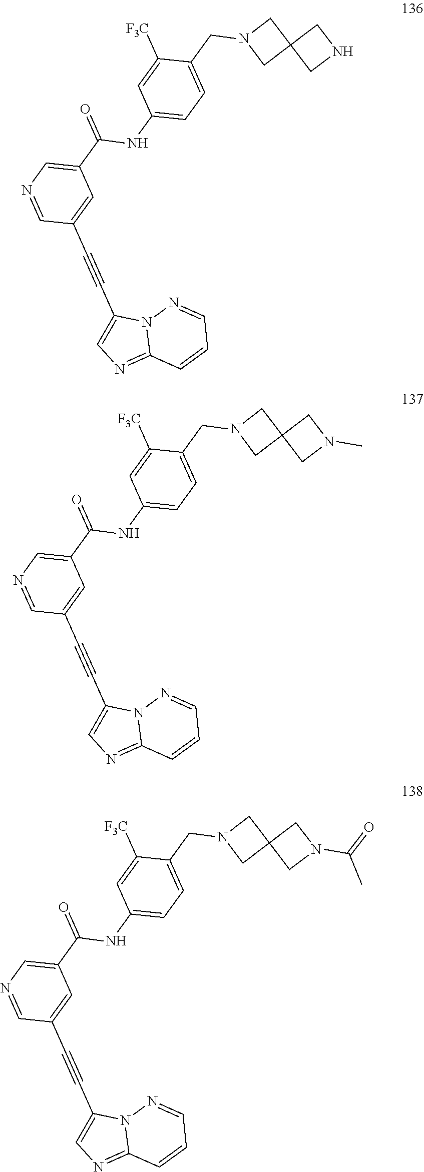 [SMILES: O=C(Nc1ccc(CN2CC3(CNC3)C2)c(C(F)(F)F)c1)c1cncc(C#Cc2cnc3cccnn23)c1 ; CN1CC2(C1)CN(Cc1ccc(NC(=O)c3cncc(C#Cc4cnc5cccnn45)c3)cc1C(F)(F)F)C2 ; CC(=O)N1CC2(CN(Cc3ccc(NC(=O)c4cncc(C#Cc5cnc6cccnn56)c4)cc3C(F)(F)F)C2)C1]

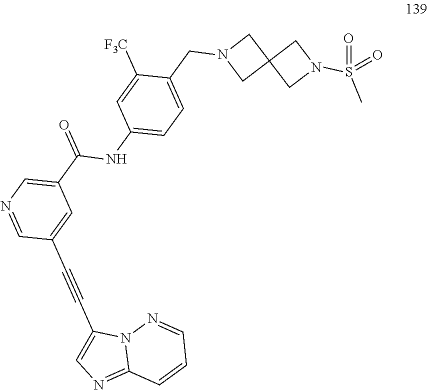 [SMILES: CS(=O)(=O)N1CC2(CN(Cc3ccc(NC(=O)c4cncc(C#Cc5cnc6cccnn56)c4)cc3C(F)(F)F)C2)C1]

These compounds are hereinafter referred to as Group 18 compounds and are illustrated in FIG. 20.

In some embodiments, the compound of the invention is:

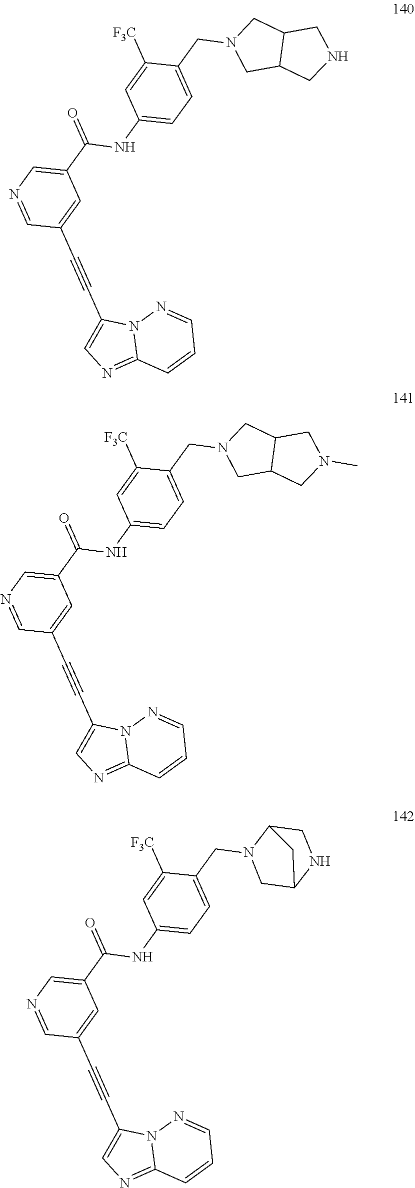 [SMILES: O=C(Nc1ccc(CN2CC3CNCC3C2)c(C(F)(F)F)c1)c1cncc(C#Cc2cnc3cccnn23)c1 ; CN1CC2CN(Cc3ccc(NC(=O)c4cncc(C#Cc5cnc6cccnn56)c4)cc3C(F)(F)F)CC2C1 ; O=C(Nc1ccc(CN2CC3CC2CN3)c(C(F)(F)F)c1)c1cncc(C#Cc2cnc3cccnn23)c1]

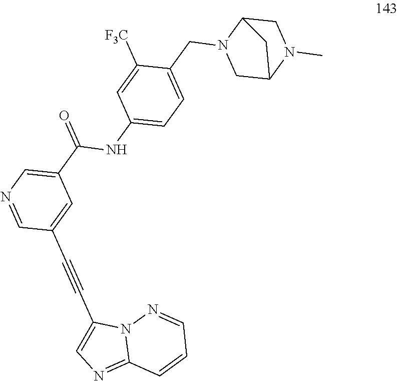 [SMILES: CN1CC2CC1CN2Cc1ccc(NC(=O)c2cncc(C#Cc3cnc4cccnn34)c2)cc1C(F)(F)F]

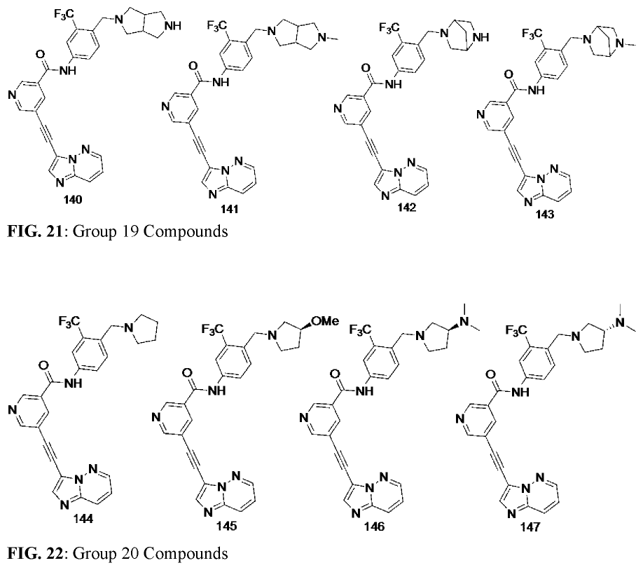

These compounds are hereinafter referred to as Group 19 compounds and are illustrated in FIG. 21.

In some embodiments, the compound of the invention is:

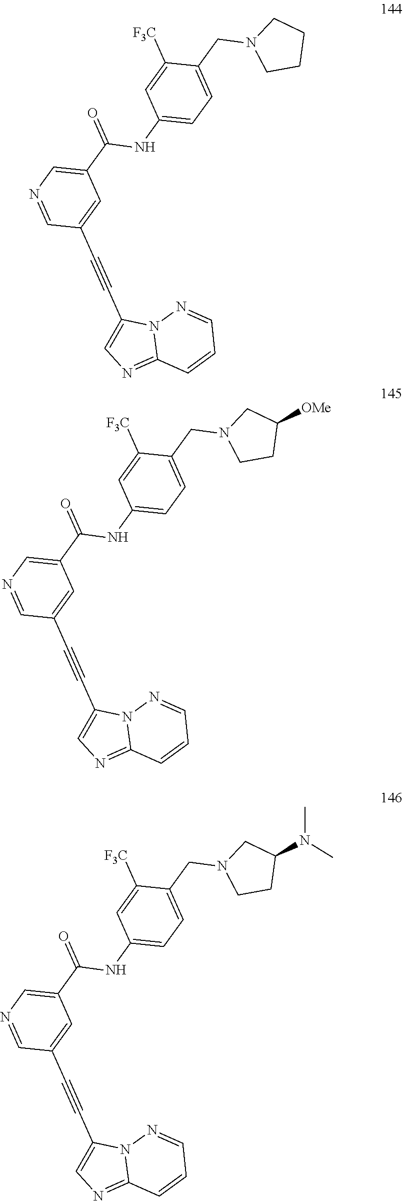 [SMILES: O=C(Nc1ccc(CN2CCCC2)c(C(F)(F)F)c1)c1cncc(C#Cc2cnc3cccnn23)c1 ; CO[C@H]1CCN(Cc2ccc(NC(=O)c3cncc(C#Cc4cnc5cccnn45)c3)cc2C(F)(F)F)C1 ; CN(C)[C@H]1CCN(Cc2ccc(NC(=O)c3cncc(C#Cc4cnc5cccnn45)c3)cc2C(F)(F)F)C1]

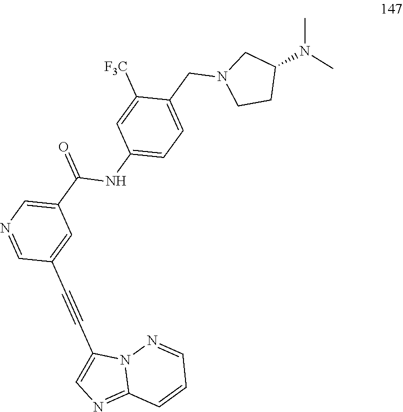 [SMILES: CN(C)[C@@H]1CCN(Cc2ccc(NC(=O)c3cncc(C#Cc4cnc5cccnn45)c3)cc2C(F)(F)F)C1]

These compounds are hereinafter referred to as Group 21 compounds and are illustrated in FIG. 22.

In some embodiments, the compound of the invention is:

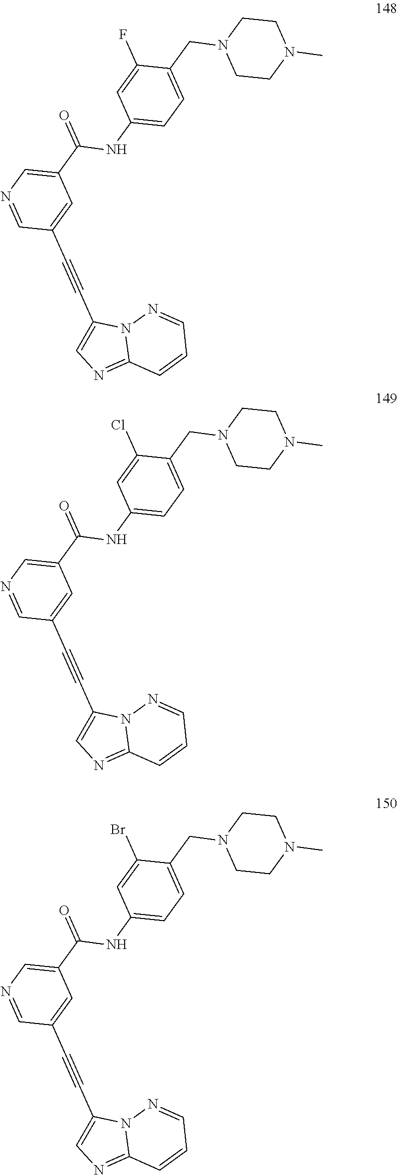 [SMILES: CN1CCN(Cc2ccc(NC(=O)c3cncc(C#Cc4cnc5cccnn45)c3)cc2F)CC1 ; CN1CCN(Cc2ccc(NC(=O)c3cncc(C#Cc4cnc5cccnn45)c3)cc2Cl)CC1 ; CN1CCN(Cc2ccc(NC(=O)c3cncc(C#Cc4cnc5cccnn45)c3)cc2Br)CC1]

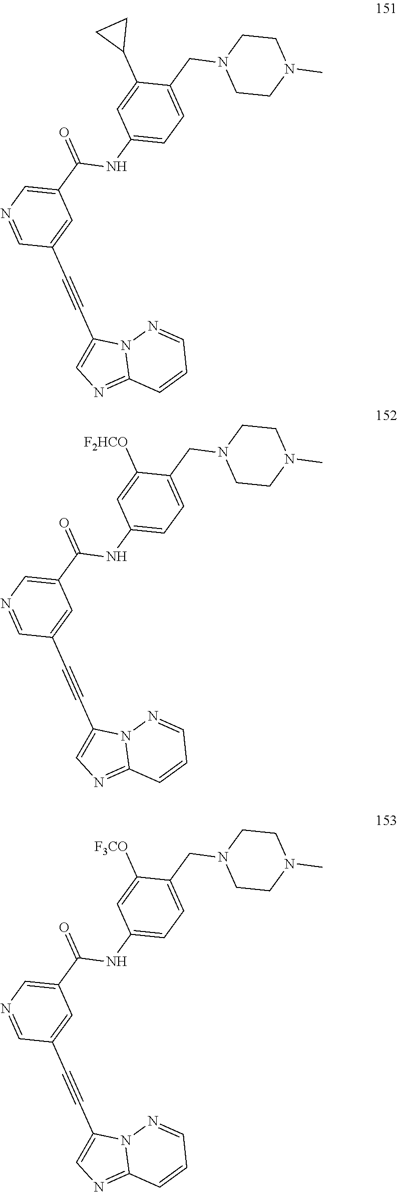 [SMILES: CN1CCN(Cc2ccc(NC(=O)c3cncc(C#Cc4cnc5cccnn45)c3)cc2C2CC2)CC1 ; CN1CCN(Cc2ccc(NC(=O)c3cncc(C#Cc4cnc5cccnn45)c3)cc2OC(F)F)CC1 ; CN1CCN(Cc2ccc(NC(=O)c3cncc(C#Cc4cnc5cccnn45)c3)cc2OC(F)(F)F)CC1]

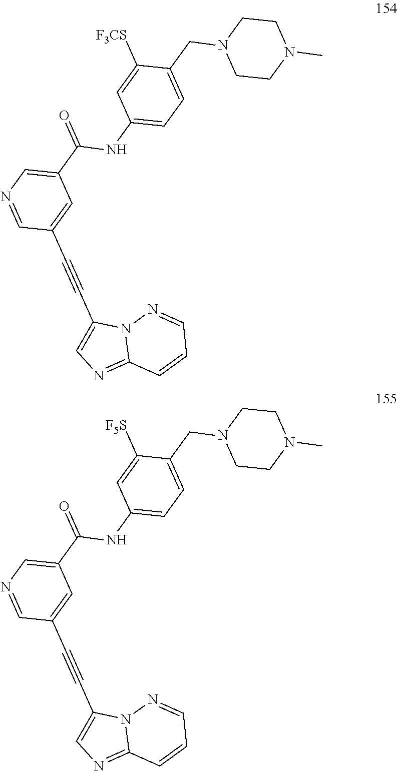

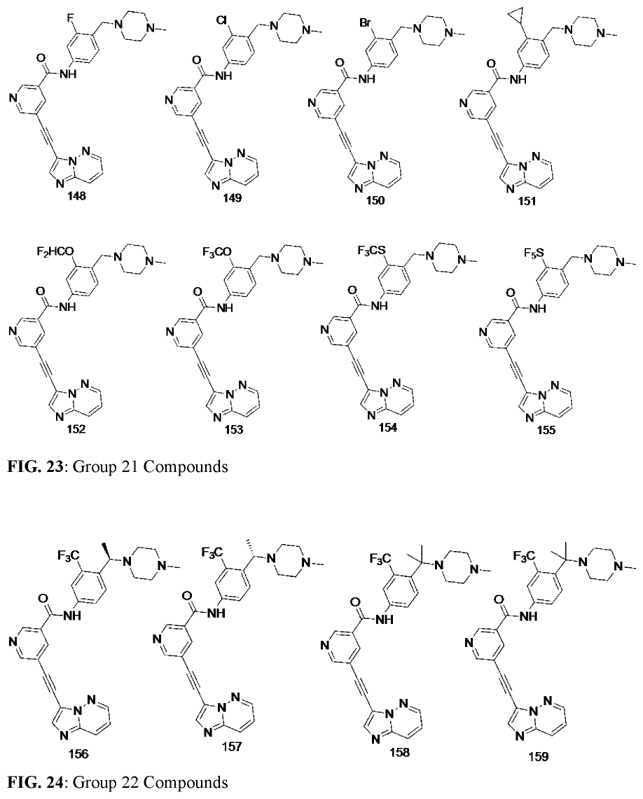

These compounds are hereinafter referred to as Group 21 compounds and are illustrated in FIG. 23.

In some embodiments, the compound of the invention is:

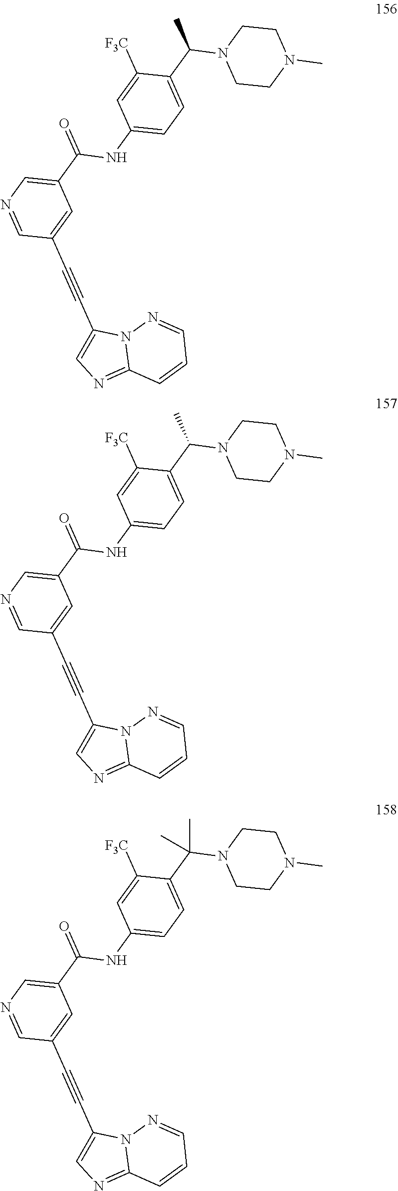

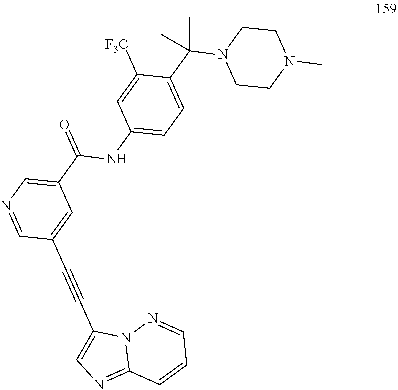 [SMILES: CN1CCN(C(C)(C)c2ccc(NC(=O)c3cncc(C#Cc4cnc5cccnn45)c3)cc2C(F)(F)F)CC1]

These compounds are hereinafter referred to as Group 22 compounds and are illustrated in FIG. 24.

In some embodiments, the compound of the invention is:

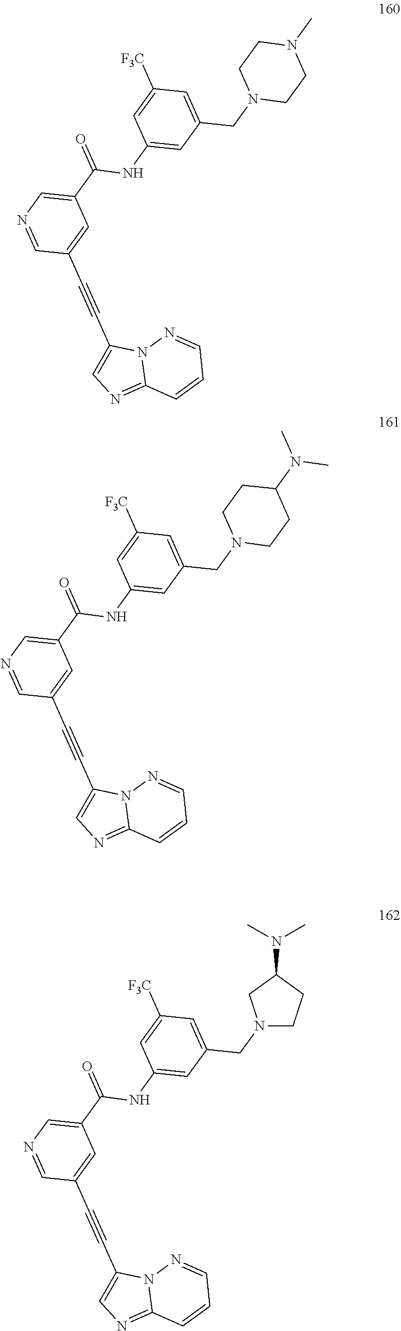 [SMILES: CN1CCN(Cc2cc(NC(=O)c3cncc(C#Cc4cnc5cccnn45)c3)cc(C(F)(F)F)c2)CC1 ; CN(C)C1CCN(Cc2cc(NC(=O)c3cncc(C#Cc4cnc5cccnn45)c3)cc(C(F)(F)F)c2)CC1 ; CN(C)[C@H]1CCN(Cc2cc(NC(=O)c3cncc(C#Cc4cnc5cccnn45)c3)cc(C(F)(F)F)c2)C1]

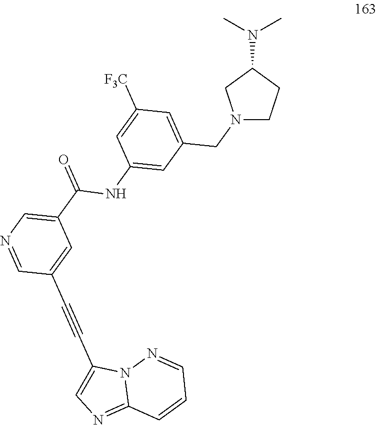 [SMILES: CN(C)[C@@H]1CCN(Cc2cc(NC(=O)c3cncc(C#Cc4cnc5cccnn45)c3)cc(C(F)(F)F)c2)C1]

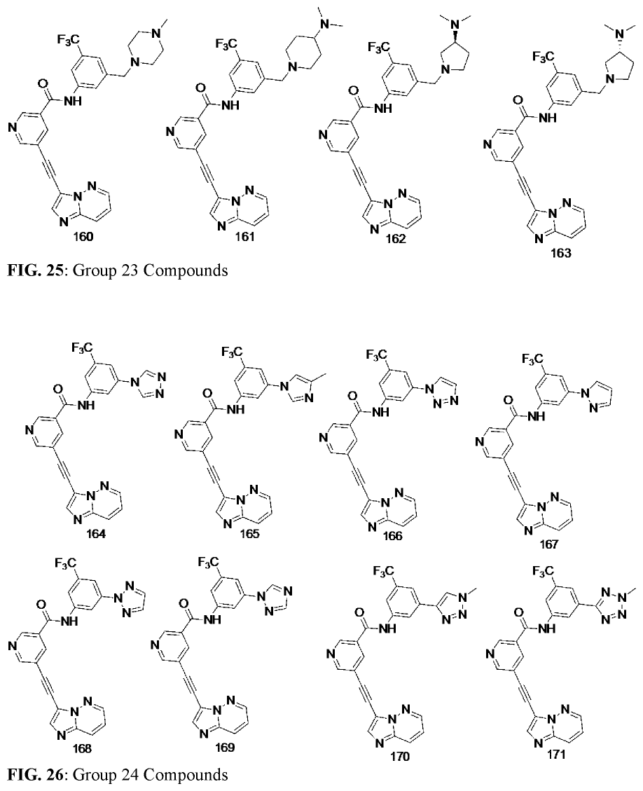

These compounds are hereinafter referred to as Group 23 compounds and are illustrated in FIG. 25.

In some embodiments, the compound of the invention is:

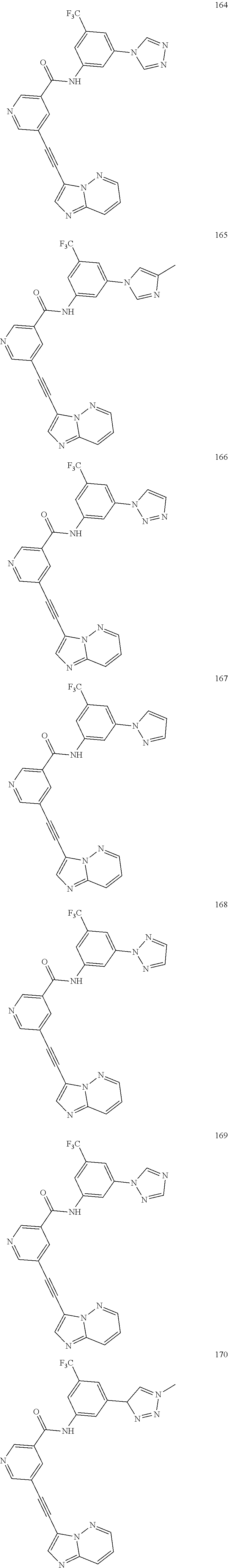

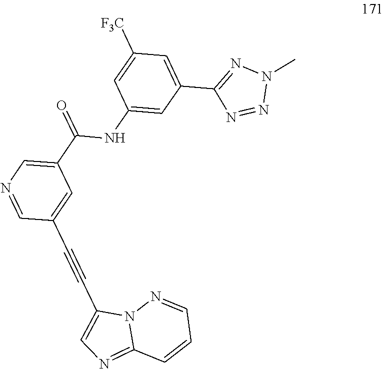 [SMILES: Cn1nnc(-c2cc(NC(=O)c3cncc(C#Cc4cnc5cccnn45)c3)cc(C(F)(F)F)c2)n1]

These compounds are hereinafter referred to as Group 24 compounds and are illustrated in FIG. 26.

In some embodiments, the compound of the invention is:

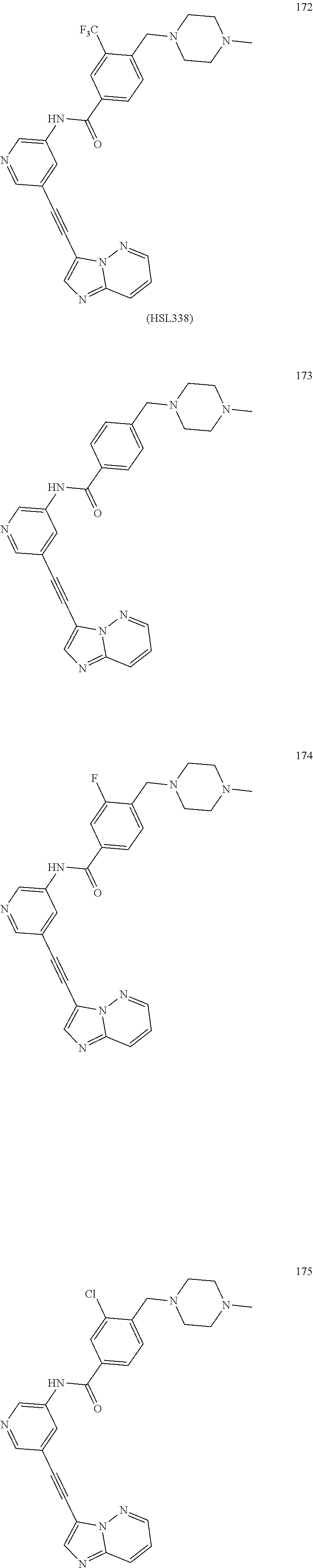 [SMILES: CN1CCN(Cc2ccc(C(=O)Nc3cncc(C#Cc4cnc5cccnn45)c3)cc2C(F)(F)F)CC1 ; CN1CCN(Cc2ccc(C(=O)Nc3cncc(C#Cc4cnc5cccnn45)c3)cc2)CC1 ; CN1CCN(Cc2ccc(C(=O)Nc3cncc(C#Cc4cnc5cccnn45)c3)cc2F)CC1 ; CN1CCN(Cc2ccc(C(=O)Nc3cncc(C#Cc4cnc5cccnn45)c3)cc2Cl)CC1]

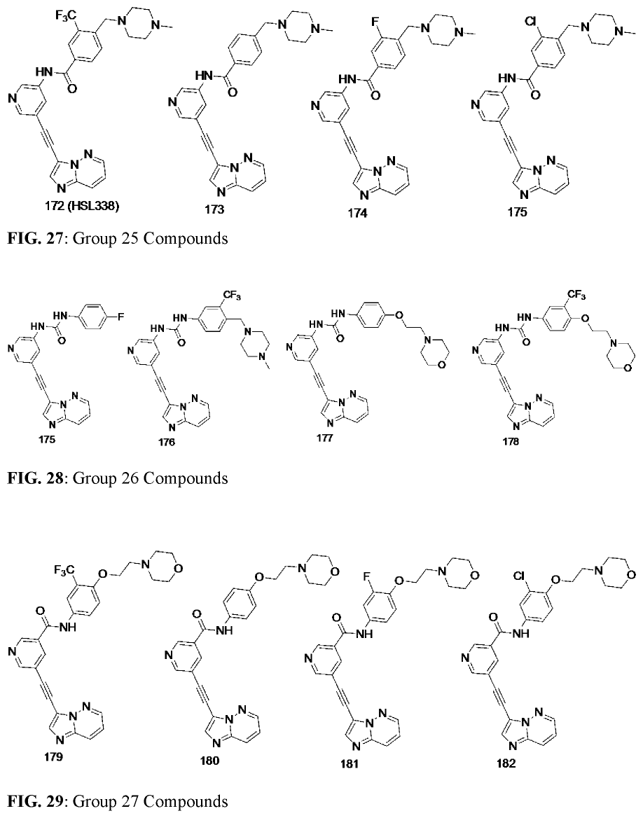

These compounds are hereinafter referred to as Group 25 compounds and are illustrated in FIG. 27.

In some embodiments, the compound of the invention is:

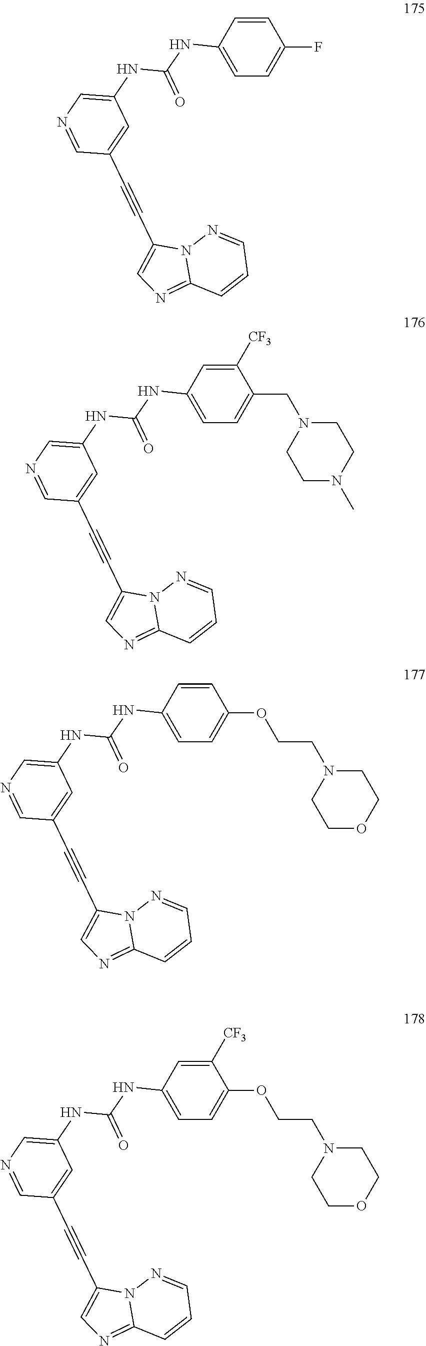

These compounds are hereinafter referred to as Group 26 compounds and are illustrated in FIG. 28.

In some embodiments, the compound of the invention is:

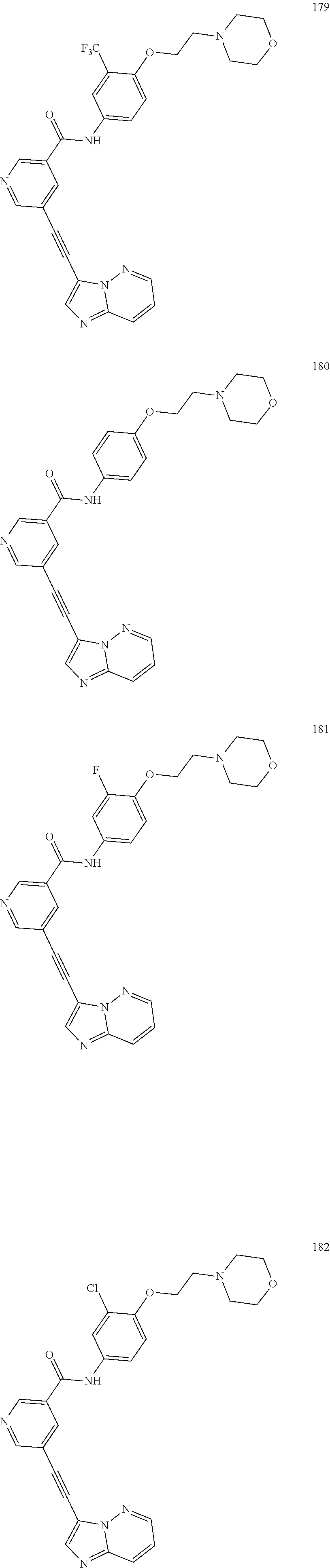 [SMILES: O=C(Nc1ccc(OCCN2CCOCC2)c(C(F)(F)F)c1)c1cncc(C#Cc2cnc3cccnn23)c1 ; O=C(Nc1ccc(OCCN2CCOCC2)cc1)c1cncc(C#Cc2cnc3cccnn23)c1 ; O=C(Nc1ccc(OCCN2CCOCC2)c(F)c1)c1cncc(C#Cc2cnc3cccnn23)c1 ; O=C(Nc1ccc(OCCN2CCOCC2)c(Cl)c1)c1cncc(C#Cc2cnc3cccnn23)c1]

These compounds are hereinafter referred to as Group 27 compounds and are illustrated in FIG. 29.

It should be appreciated that the teaching of the present invention includes Prodrugs of the above-identified compounds. Additionally, conjugates of the above-identified compounds, whereby the compounds are conjugates to a targeting agent or an agent that aids the degradation of a target, such as PROTAC strategy. It should also be appreciated that Polymorphic salt forms of the disclosed compounds, taught by WO 2015/001098 A1 (The entire contents and disclosures of this patent application are incorporated herein by reference) and variants thereof, whereas the pharmaceutically acceptable salt could be HCl, acetate, sulfate, phosphate, citrate, and other salts obvious to one skilled in the art are within the scope of the present invention.

In some embodiments, the protein kinase inhibited by claimed compounds is one known in the art. In some embodiments, the protein kinase includes, but is not limited to, FLT3 and Haspin. In some embodiments, the protein kinase is Abl, Abl2, AFK, ALK, AKT1, AMPK_group, ATM, ATR, Aurora A, Aurora B, Aurora C, Axl, BCKDK, BLK, BMPR1B, BMX, BRAF, Brk, BRSK1, BTK, CaM-Klalpha, CaM-Kllalpha, CaMKK_group, CaM-KIV, CaM-KKalpha, CaM-KKbeta, CCDPK, CCRK, CDK1, CDK11, CDK2, CDK4, CDK5, CDK6, CDK7, CDK8, CDK9, CDK18, CDK19, CDK_group, CDPK, Chak1, CHK1, CHK2, CK1 alpha, CK1 delta, CK1 epsilon, CK1_group, CK2 alpha, CK2_beta, CK2_group, CLK1, CSF1R, Csk, DAPK1, DAPK2, DAPK3, DAPK_group, DCAMKL1, DMPK_group, DNA-PK, DYRK1A, DYRK1B, DYRK2, DYRK3, eEF2K, Eg3 kinase, EGFR, EIF2AK2, EphA2, EphA3, EphA4, EphA8, EphB1, EphB2, EphB3, EphB5, ErbB2, ERK1, ERK2, ERK5, ERK7, ERN1/IRE1, FAK, Fer, Fes, FGFR1, FGFR3, FGFR4, FGFR_group, Fgr, FLT1, FLT3, FLT4, Fyn, GRK-1, GRK-2, GRK-3, GRK-4, GRK-5, GRK-6, GRK_group, GSK-3alpha, GSK-3beta, GSK-3_group, HER2, HER4, HCK, HIPK2, HIPK3, HRI, ICK, IGF1R, IKK-alpha, IKK-beta, IKK-epsilon ILK, InsR, IPL1, IRAK1, IRAK4, ITK, JAK1, JAK2, JAK3, JAK_group, JNK_group, KDR, KIS, Kit, KSR1, Lck, LIMK1, LIMK2, LKB1, LOK, LRRK2, Lyn, MAP2K1, MAP2K2, MAP2K3, MAP2K4, MAP2K6, MAP2K7, MAPK2_group, MAP3K1, MAP3K11, MAP3K14, MAP3K5, MAP3K7, MAP3K8, MAPK3_group, MAP4K1, MAP4K2, MAP4K4, MAPK1, MAPK10, MAPK11, MAPK12, MAPK13, MAPK14, MAPK3, MAPK4, MAPK6, MAPK7, MAPK8, MAPK9, MAPK_group, MAPKAPK2, MARK_group, Mer, MEK1, MEK2, Met, MERTK, MHCK, MLCK_group, MLKL, MK2, Mnk1, Mnk2, MOS, MRCKα, MST1, MST3, mTOR, NDR1, NDR2, NEK1, NEK2, NEK6, NEK9, NEK_group, NLK, NuaK1, p37 kinase, p38_group, p70S6K, p70S6Kb, PBK/TOPK, P70S6K_group, PAK1, PAK2, PAK3, PAK5, PAK6, PAK_group, PASK, P-CIP2, PCTAIRE1, PDGFR alpha, PDGFR beta, PDGFR_group, PDHK1, PDHK2, PDHK3, PDHK4, PDK-1, PDK-2, PDK_group, PHK_group, PIK3CA, PIK3CB, PIK3CD, PIK3CG, Pim-1, PKA alpha, Pka_group, PKB beta, PKB_group, PKC alpha, PKC beta, PKC delta, PKC epsilon, PKC eta, PKC gamma, PKC iota, PKC theta, PKC zeta, PKC_group, PKD1, PKD2, PKD3, PKG1/cGK-I, PKG2/cGK-II, PKG2/cGK_group, PKN1, PLK1, PLK2, PLK3, PRP4, PYK2, RAF1, Ret, RIPK1, RIPK2, RIPK3, RIPK4, ROCK1, ROCK2, Ron, ROS, RPL10, RSK-1, RSK-2, RSK-3, RSK-5, SDK1, SGK_group, SIK, Sky, Src, Src_group, STLK3, Syk, TBK1, Tec, TESK1, TESK2, TGFbR1, TGFbR2, Tie1, Tie2, Titin kinase, TNK2, TRKA, TRKB, TRKC, tropomyosin kinase, TSSK3, TXK, Tyk2, TYK2, ULK1, ULK2, VRK1, Wee1, Wnk1, WNK1, Yes, ZAP70.

The present invention is directed to a pharmaceutical composition comprising one or more compounds as described herein, or a pharmaceutically acceptable salt, N-oxide, hydrate, solvate, tautomer, or optical isomer thereof, and a pharmaceutically acceptable carrier or diluent.

In yet another aspect, the present invention is directed to a method of treating, inhibiting, suppressing, or reducing the severity of cancer in a subject in need thereof, wherein the method comprises administering to the subject a therapeutically effective amount of a compound as described herein, or a pharmaceutically acceptable salt, N-oxide, hydrate, solvate, tautomer, or optical isomer thereof, or a pharmaceutical composition containing one or more compounds as described herein.

In yet another aspect, the present invention is directed to a method of treating, inhibiting, suppressing, or reducing the severity of a disease or a disorder associated with protein kinase in a subject in need thereof, wherein the method comprises administering to the subject a therapeutically effective amount of a compound as described herein, or a pharmaceutically acceptable salt, N-oxide, hydrate, solvate, tautomer, or optical isomer thereof, or a pharmaceutical composition containing one or more compounds as described herein.

### EXAMPLES

### Example 1

Ponatinib is more promiscuous against cardiovascular related kinases than HSN748

Ponatinib and HSN748 were screened against various disease-associated kinases, which have been shown to be inhibited by ponatinib (see Table 1). Interestingly the inhibition profile of HSN748 against ABL1(T315I) and FLT3-ITD were similar to ponatinib but there were some notable differences with some kinases.

TABLE 1
IC50 of ponatinib and HSN748 against several kinases

IC50 (nM)^{a}

| Kinase | HSN748 | Ponatinib |
|---|---|---|
| ABL1 | 1.1 | 0.87 |
| ABL1 (T315I) | 11.1 | 2.5 |
| c-SRC | >1000 | 4.6 |
| FGFR1 | 24.4 | 6.9 |
| FGFR2 | 11.7 | 6.0 |
| FGFR3 | 96.4 | 25.0 |
| FGFR4 | 125.1 | 46.8 |
| FLT3 (D835Y) | 13.8 | 176 |
| FLT3 (ITD) | 1.5 | 10.03 |
| P38a/MAPK14 | 382.7 | 86.6 |
| p70S6K/RPS6KB1 | 273.1 | 200.1 |
| PDGFRa | 10.7 | 3.8 |
| PDGFRb | 10.2 | 7.01 |
| RET | 0.65 | 0.88 |
| MNK1 | 202 | 3930 |
| MNK2 | 9.36 | 268 |

^{a}IC50 was determined at Reaction Biology (Malvern, PA). [ATP] = 100 μM

HSN748 was inactive against c-Src kinase (IC50>1 μM) while ponatinib potently inhibited c-Src (IC₅₀ of 4.6 nM), see Table 1. Src has been shown to play various roles in heart function. For example, Src plays critical roles in maintaining the structure of myocyte¹⁴. Recently it was also revealed that Src regulates hERG current amplitude¹⁵. Hence the inhibition of Src might lead to the disorganization of myofibrils as well as affecting cardiac ion channels. Src is also abundantly found in human platelets and it is key to signal initiation and propagation from αIIbβ3, which is one of the most abundant receptors found on platelets^{16,17}.

Thus, despite the oncogenic role played by Src in various cancers, its inhibition could come with the dysregulation of normal cells and platelets. It is therefore noteworthy that HSN748 does not inhibit Src as potently as ponatinib does.

Ponatinib has been shown to inhibit FGFRs and it is currently undergoing clinical trials for the treatment of biliary cancer with FGFR2 fusion⁷. Although many drugs that target FGFRs are currently undergoing clinical trials, FGFRs have important cardiac and liver functions and so their inhibitions could lead to adverse events. Hyperphosphatemia is one major complication that is associated with FGFR inhibition due to interruption to FGF23 signaling¹⁸. Pan-FGFR inhibition has been linked to cardiovascular dysfunction¹⁹. Ponatinib is more active in vitro against FGFR1-4 than HSN748.

ABL1 and FLT3 are mutated in CML and AML respectively. Ponatinib and HSN748 have similar activities against ABL1, ABL1(T315I) and FLT3-ITD. Interestingly HSN748 has a significantly lower IC₅₀ against FLT3(D835Y) kinase than ponatinib (compare IC50 of 14 nM for HSN748 versus 173 nM for ponatinib, see Table 2). Most FLT3 inhibitors used in the clinic show initial efficacy but within months patients relapse due to kinase mutation, which reduces the efficacy of treatment²⁰. The D835 mutation is one of the most frequent mutations observed at in a study using the TKI quizartinib²¹. Thus for drug-resistant AML (due to kinase mutation), HSN748 could be a better treatment option than ponatinib.

### Example 2

HSN748 Potently Inhibits AML Cell Lines Better than Ponatinib

TABLE 2
Activities of HSN748 and ponatinib against
FLT3, ABL1, FGFR and RET-driven cancers.

IC50 (nM)

| Cell Line | HSN748 | Ponatinib |
|---|---|---|
| MV-4-11 | 0.07 | 0.09 |
| MOLM13-res | 1.73 | 24.1 |
| MOLM14 | 0.05 | 0.45 |
| MOLM14-D835Y | 0.69 | 52.6 |
| MOLM14-F691L | 0.18 | 6.8 |
| K562 | 0.8 | 0.6 |
| KCL22 | 1.32 | 0.14 |
| KCL22-IR | 0.23 | 0.66 |
| LC2/Ad | 41 | 35.1 |
| H520 | 838.6 | 128.2 |

We proceeded to test whether the degree of inhibition of FLT3, ABL1, RET and FGFR-driven cancers by ponatinib and HSN748 mirrored the order of kinase inhibition. The IC50 for growth inhibition by both compounds against MV4-11 (FLT3), K562 (ABL1) and LC2/ad (RET) were similar. HSN748 was better at inhibiting quizartinib-resistant AML (MOLM14-D835Y cell line) than ponatinib (compare IC50 of 0.69 nM for HSN748 and 52.6 nM for ponatinib). For gilteritinib-resistant AML cell line, Molm14 (ITD, F691L), HSN748 was also more potent than ponatinib (IC50 of 0.18 nM for HSN748 and 6.8 nM for ponatinib).

Cell activities of other nicotinamide compounds:

| | IC₅₀ (nM) | | | | | |
|---|---|---|---|---|---|---|
| | | | | | MOLM-14 | MOLM-14 |
| | | | | | (ITD, | (ITD, |
| | K562 | KCL22 | KCL22-IR | MOLM-14 | D85Y) | F691L) |
| HSL420 | 0.059 ± 0.002 | 0.06 ± 0.002 | 0.46 ± 0.01 | — | 2.24 ± 0.08 | 0.57 ± 0.004 |
| HSL381 | 0.57 ± 0.003 | 0.89 ± 0.005 | 4.24 ± 0.05 | 0.04 ± 0.006 | 7.12 ± 0.06 | 0.55 ± 0.06 |
| HSL382 | Above 100 nM | Above 100 nM | Above 100 nM | 5.15 ± 0.09 | Above 100 nM | Above 100 nM |
| HSL385 | 0.94 ± 0.002 | 2.62 ± 0.06 | 17.2 ± 0.8 | 0.15 ± 0.007 | 28.5 ± 0.9 | — |
| HSL338 | 0.45 ± 0.002 | 1.4 ± 0.02 | 4.2 ± 0.04 | 0.075 ± 0.0005 | 7.4 ± 0.04 | — |
| HSL412 | 28.1 ± 0.6 | 28.1 ± 0.4 | — | — | Above 100 nM | 17.84 ± 1.3 |
| HSL407 | 1.83 ± 0.02 | 2.93 ± 0.04 | 49.7 ± 2.5 | — | 42.9 ± 0.7 | 6.87 ± 0.13 |

### Compound Characterization

HSN748: 5-(imidazo[1,2-b]pyridazin-3-ylethynyl)-N-(4-((4-methylpiperazin-1-yl)methyl)-3-(trifluoromethyl)phenyl)nicotinamide

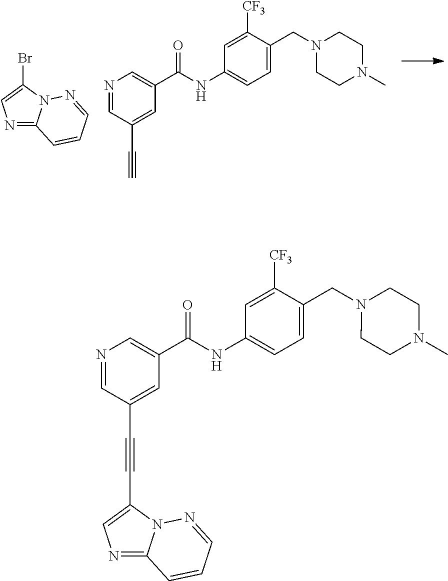

A solution of bromo compound (72 mg, 0.37 mmol, 1 equiv), Pd(PPh₃)₄ (10 mol %), CuI (5 mol %) and Triphenylphosphine (10 mg) in Triethylamine (1.5 mL, 10.78 mmol, 29.13 equiv) was deoxygenated using argon gas. A deoxygenated solution of alkyne (171 mg, 0.43 mmol, 1.2 equiv) in DMF (4 mL) was added over 10 minutes to the solution. The reaction temperature was then increased to 55° C. and allowed to stir for 12 h. The reaction was diluted with ethyl acetate (300 mL). The organic layer was washed with water (5×50 mL), saturated NH₄Cl (1×50 mL) and brine (1×50 mL). Combined organic layers were dried over anhydrous sodium sulphate, filtered and concentrated in vacuo. The pure product was obtained via flash column chromatography. Yield: 100 mg, 53%; TLC R_{f}: 0.2 (10% MeOH/CH₂Cl₂)

¹H NMR (500 MHz, DMSO-d₆) δ 10.77 (s, 1H), 9.10 (d, J=2.2 Hz, 1H), 8.97 (d, J=2.0 Hz, 1H), 8.72 (d, J=2.9 Hz, 1H), 8.55 (s, 1H), 8.27 (d, J=8.6 Hz, 2H), 8.19 (s, 1H), 8.03 (d, J=8.4 Hz, 1H), 7.72 (d, J=8.5 Hz, 1H), 7.41 (dd, J=9.2, 4.5 Hz, 1H), 3.56 (s, 2H), 2.38 (s, 8H), 2.16 (s, 3H); ¹³C NMR (126 MHz, DMSO-d₆) δ 163.74, 154.01, 149.01, 145.57, 140.32, 139.44, 138.26, 137.60, 133.01, 131.82, 130.29, 126.67, 124.00, 119.90, 119.04, 117.72, 111.59, 94.86, 81.09, 57.88, 55.13, 53.06, 46.08; HRMS (ESI⁺) calcd. for C₂₇H₂₅F₃N₇O (MH⁺) 520.2067, found 520.2066

HSL338: N-(5-(imidazo[1,2-b]pyridazin-3-ylethynyl)pyridin-3-yl)-4-((4-methylpiperazin-1-yl)methyl)-3-(trifluoromethyl)benzamide

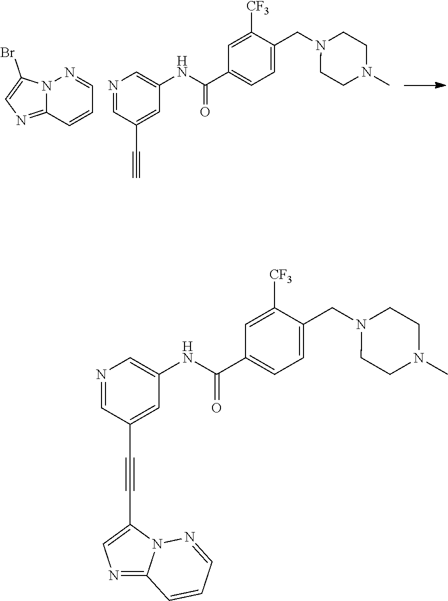

A solution of bromo compound (80 mg, 0.404 mmol, 1 equiv), Pd(PPh₃)₂Cl₂ (10 mol %), CuI (5 mol %) and Triphenylphosphine (10.5 mg, 0.04 mmol, 10 mol %) in triethylamine (1.5 mL, 10.78 mmol, 26.6 equiv) was deoxygenated using argon gas. A deoxygenated solution of alkyne (194.9 mg, 0.484 mmol, 1.2 equiv) in DMF (4 mL) was slowly added over 10 minutes. The reaction was then moved to 50° C. and allowed to run for 15 hrs. The reaction was the cooled to room temperature, diluted with ethyl acetate (150 mL) and washed with water (5×50 mL) and brine (1×50 mL). Combined organic layers were concentrated in vacuo and purified via column chromatography. Yield: 72.1 mg 28.6%

¹H NMR (500 MHz, DMSO-d₆) δ 10.76 (s, 1H), 8.97-8.90 (m, 1H), 8.71 (dd, J=4.4, 1.5 Hz, 1H), 8.57-8.51 (m, 1H), 8.45 (t, J=2.2 Hz, 1H), 8.28 (d, J=1.8 Hz, 1H), 8.25 (ddd, J=8.4, 5.4, 1.7 Hz, 3H), 7.95 (d, J=8.1 Hz, 1H), 7.40 (dd, J=9.2, 4.4 Hz, 1H), 3.69 (s, 2H), 2.50-2.37 (m, 8H), 2.26 (s, 3H). ¹³C NMR (126 MHz, DMSO-d₆) δ 165.1, 146.5, 145.5, 142.0, 139.2, 135.9, 133.5, 132.2, 131.3, 129.2, 128.0, 127.7, 126.6, 125.6, 123.5, 119.8, 119.0, 111.8, 95.3, 80.2, 57.8, 54.8, 52.7, 45.6, 40.5, 40.3, 40.1, 40.0, 39.8, 39.6, 39.5.

HSL381: 5-(imidazo[1,2-a]pyridin-3-ylethynyl)-N-(4-((4-methylpiperazin-1-yl)methyl)-3-(trifluoromethyl)phenyl)nicotinamide

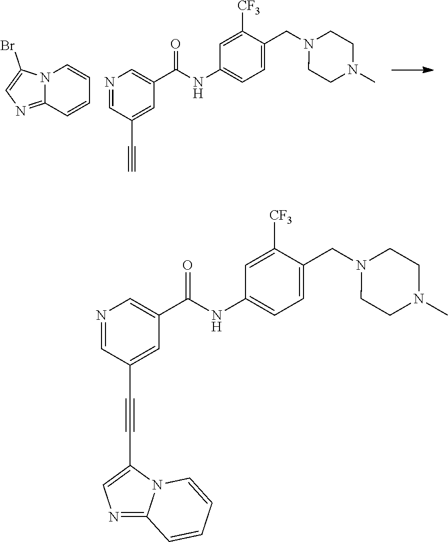

A solution of bromo compound (75 mg, 0.381 mmol, 1 equiv), Pd(PPh₃)₂Cl₂ (10 mol %), CuI (5 mol %) and Triphenylphosphine (10.5 mg, 0.04 mmol, 10 mol %) in triethylamine (1.5 mL, 10.78 mmol, 28.29 equiv) was deoxygenated using argon gas. A deoxygenated solution of alkyne (183.6 mg, 0.456 mmol, 1.2 equiv) in DMF (4 mL) was slowly added over 10 minutes. The reaction was then moved to 50° C. and allowed to run for 15 hrs. The reaction was the cooled to room temperature, diluted with ethyl acetate (150 mL) and washed with water (5×50 mL) and brine (1×50 mL). Combined organic layers were dried over anhydrous sodium sulphate, concentrated in vacuo, and purified via column chromatography. Yield: 47.8 mg, 24.2%

¹H NMR (500 MHz, DMSO-d₆) δ 10.81 (s, 1H), 9.11-9.02 (m, 2H), 8.80 (d, J=6.7 Hz, 1H), 8.62 (d, J=2.2 Hz, 1H), 8.21 (d, J=2.2 Hz, 1H), 8.09 (s, 1H), 8.05 (dd, J=8.5, 2.2 Hz, 1H), 7.73 (t, J=9.4 Hz, 2H), 7.46 (dd, J=9.0, 6.7 Hz, 1H), 7.17 (t, J=6.7 Hz, 1H), 3.58 (s, 3H), 2.48 (s, J=1.7 Hz, 8H), 2.25 (s, 3H). ¹³C NMR (126 MHz, DMSO-d₆) δ 164.0, 153.9, 148.4, 139.5, 138.3, 137.4, 132.8, 131.8, 130.3, 128.1, 127.8, 127.5, 126.7, 125.8, 124.0, 123.6, 119.4, 118.0, 117.7, 114.5, 96.0, 81.4, 57.7, 54.8, 52.5, 45.5, 40.5, 40.3, 40.1, 40.0, 39.8, 39.6, 39.5.

HSL382: 5-(imidazo[1,2-a]pyrimidin-3-ylethynyl)-N-(4-((4-methylpiperazin-1-yl)methyl)-3-(trifluoromethyl)phenyl)nicotinamide

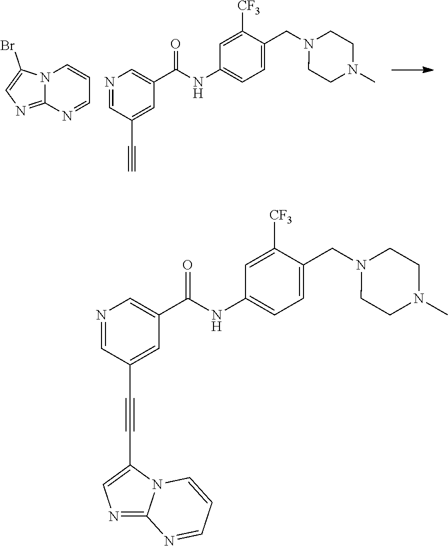

A solution of bromo compound (75 mg, 0.378 mmol, 1 equiv), Pd(PPh₃)₂Cl₂ (10 mol %), CuI (5 mol %) and Triphenylphosphine (10.5 mg, 0.04 mmol, 10 mol %) in triethylamine (1.5 mL, 10.78 mmol, 28.5 equiv) was deoxygenated using argon gas. A deoxygenated solution of alkyne (194.9 mg, 0.484 mmol, 1.2 equiv) in DMF (4 mL) was slowly added over 10 minutes. The reaction was then moved to 50° C. and allowed to run for 15 hrs. The reaction was the cooled to room temperature, diluted with ethyl acetate (150 mL) and washed with water (5×50 mL) and brine (1×50 mL). Combined organic layers were dried over anhydrous sodium sulphate, concentrated in vacuo and purified via column chromatography. Yield: 57.6 mg, 29.3%

¹H NMR (500 MHz, DMSO-d₆) δ 10.92 (s, 1H), 9.28 (dd, J=6.8, 2.0 Hz, 1H), 9.12 (d, J=2.2 Hz, 1H), 9.06 (d, J=2.0 Hz, 1H), 8.71 (dt, J=4.2, 2.4 Hz, 2H), 8.24 (d, J=2.2 Hz, 2H), 8.08 (dd, J=8.5, 2.2 Hz, 1H), 7.72 (d, J=8.5 Hz, 1H), 7.30 (dd, J=6.7, 4.2 Hz, 1H), 3.57 (s, 3H), 2.42 (s, 8H), 2.22 (s, 3H). ¹³C NMR (126 MHz, DMSO-d₆) δ 163.9, 153.9, 152.6, 149.0, 148.7, 140.4, 138.4, 137.8, 135.3, 132.8, 131.8, 130.2, 128.0, 127.8, 124.1, 123.6, 119.1, 117.8, 110.8, 106.4, 95.7, 80.6, 57.8, 54.9, 52.7, 45.7, 40.5, 40.3, 40.1, 40.0, 39.8, 39.6, 39.5.

HSL385: 5-(imidazo[1,2-a]pyrazin-3-ylethynyl)-N-(4-((4-methylpiperazin-1-yl)methyl)-3-(trifluoromethyl)phenyl)nicotinamide

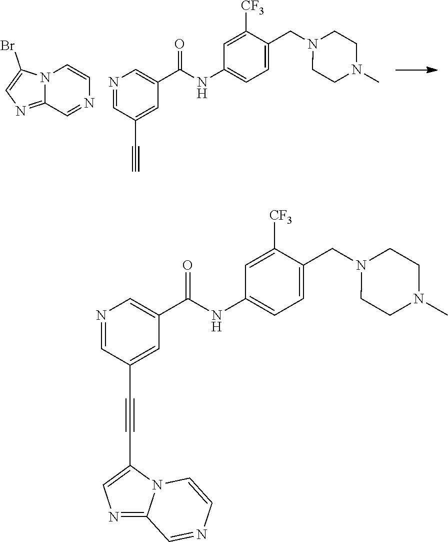

A solution of bromo compound (70 mg, 0.353 mmol, 1 equiv), Pd(PPh₃)₂Cl₂ (10 mol %), CuI (5 mol %) and Triphenylphosphine (10.5 mg, 0.04 mmol, 10 mol %) in triethylamine (1.5 mL, 10.78 mmol, 30.5 equiv) was deoxygenated using argon gas. A deoxygenated solution of alkyne (170 mg, 0.424 mmol, 1.2 equiv) in DMF (4 mL) was slowly added over 10 minutes. The reaction was then moved to 50° C. and allowed to run for 15 hrs. The reaction was the cooled to room temperature, diluted with ethyl acetate (150 mL) and washed with water (5×50 mL) and brine (1×50 mL). Combined organic layers were dried over anhydrous sodium sulphate, concentrated in vacuo and purified via column chromatography. Yield: 71.9 mg, 39.2%

¹H NMR (500 MHz, DMSO-d₆) δ 10.85 (s, 1H), 9.21 (d, J=1.5 Hz, 1H), 9.11 (dd, J=18.4, 2.1 Hz, 2H), 8.88 (dd, J=4.5, 1.5 Hz, 1H), 8.69 (d, J=2.2 Hz, 1H), 8.28 (s, 1H), 8.21 (d, J=2.2 Hz, 1H), 8.14 (d, J=4.5 Hz, 1H), 8.05 (dd, J=8.4, 2.2 Hz, 1H), 7.72 (d, J=8.5 Hz, 1H), 3.57 (s, 3H), 2.48-2.28 (m, 8H), 2.23 (s, 3H). ¹³C NMR (126 MHz, DMSO-d₆) δ 163.9, 154.1, 148.9, 143.7, 140.9, 140.4, 138.3, 137.9, 132.9, 131.8, 131.3, 130.3, 128.0, 127.8, 125.8, 124.0, 123.6, 119.9, 118.8, 117.8, 108.9, 96.7, 79.8, 57.8, 54.9, 52.7, 45.7, 40.5, 40.3, 40.1, 40.0, 39.8, 39.6, 39.5.

HSL407: 5-(imidazo[1,2-a]pyrazin-5-ylethynyl)-N-(4-((4-methylpiperazin-1-yl)methyl)-3-(trifluoromethyl)phenyl)nicotinamide

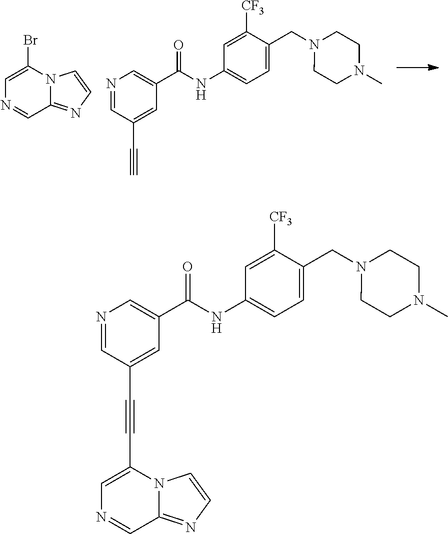

A solution of bromo compound (56 mg, 0.283 mmol, 1 equiv), Pd(PPh₃)₂Cl₂ (10 mol %), CuI (5 mol %) and Triphenylphosphine (7.86 mg, 0.03 mmol, 10 mol %) in triethylamine (1.5 mL, 10.78 mmol, 38.1 equiv) was deoxygenated using argon gas. A deoxygenated solution of alkyne (137 mg, 0.341 mmol, 1.2 equiv) in DMF (4 mL) was slowly added over 10 minutes. The reaction was then moved to 50° C. and allowed to run for 15 hrs. The reaction was the cooled to room temperature, diluted with ethyl acetate (150 mL) and washed with water (5×50 mL) and brine (1×50 mL). Combined organic layers were dried over anhydrous sodium sulphate, concentrated in vacuo and purified via column chromatography. Yield:125.8 mg, 85.6%

¹H NMR (500 MHz, DMSO-d₆) δ 10.87 (s, 1H), 9.17 (d, J=2.0 Hz, 3H), 8.76 (q, J=2.5 Hz, 1H), 8.57 (s, 1H), 8.32 (s, 1H), 8.21 (d, J=2.2 Hz, 1H), 8.07-8.00 (m, 2H), 7.72 (d, J=8.5 Hz, 1H), 3.57 (s, 3H), 2.47-2.27 (m, 8H), 2.22 (s, 3H). ¹³C NMR (126 MHz, DMSO-d₆) δ 163.8, 154.8, 149.7, 143.7, 140.0, 138.7, 138.3, 136.8, 134.2, 133.0, 131.8, 130.4, 128.1, 125.8, 124.0, 117.9, 117.7, 115.4, 114.9, 96.8, 82.9, 57.8, 54.9, 52.7, 45.7, 40.5, 40.3, 40.1, 40.0, 39.8, 39.6.

HSL420: 5-(imidazo[1,2-b]pyridazin-3-ylethynyl)-6-methyl-N-(4-((4-methylpiperazin-1-yl)methyl)-3-(trifluoromethyl)phenyl)nicotinamide

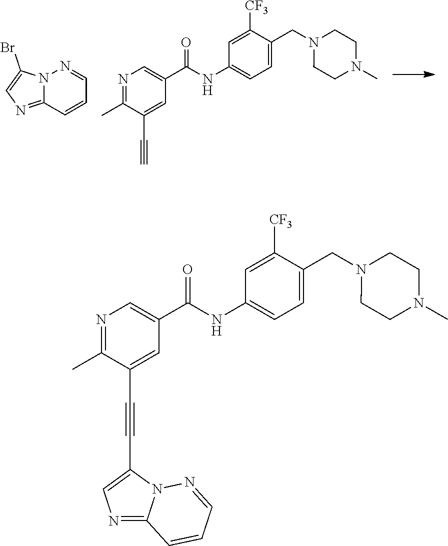

A solution of bromo compound (100 mg, 0.510 mmol, 1 equiv), Pd(PPh₃)₂Cl₂ (10 mol %), CuI (5 mol %) and Triphenylphosphine (13.1 mg, 0.05 mmol, 10 mol %) in triethylamine (1.5 mL, 10.78 mmol, 21.1 equiv) was deoxygenated using argon gas. A deoxygenated solution of alkyne (254.6 mg, 0.612 mmol, 1.2 equiv) in DMF (4 mL) was slowly added over 10 minutes. The reaction was then moved to 50° C. and allowed to run for 15 hrs. The reaction was the cooled to room temperature, diluted with ethyl acetate (150 mL) and washed with water (5×50 mL) and brine (1×50 mL). Combined organic layers were dried over anhydrous sodium sulphate, concentrated in vacuo and purified via column chromatography. Yield: 104.5 mg, 38.4%

¹H NMR (500 MHz, DMSO-d₆) δ 10.69 (s, 1H), 8.99 (d, J=2.2 Hz, 1H), 8.73 (dd, J=4.3, 2.2 Hz, 1H), 8.49 (d, J=2.3 Hz, 1H), 8.28-8.24 (m, 2H), 8.19 (d, J=2.5 Hz, 1H), 8.04 (d, J=8.4 Hz, 1H), 7.70 (d, J=8.5 Hz, 1H), 7.40 (ddd, J=9.1, 4.5, 1.9 Hz, 1H), 3.56 (s, 2H), 2.80 (s, 3H), 2.41 (s, 8H), 2.21 (s, 3H). ¹³C NMR (126 MHz, DMSO-d₆) δ 163.8, 162.7, 148.4, 145.6, 140.3, 139.1, 138.4, 137.7, 132.8, 131.8, 128.0, 127.8, 126.7, 124.0, 119.8, 117.8, 111.8, 95.2, 83.7, 57.8, 55.0, 52.8, 45.8, 40.5, 40.3, 40.2, 40.0, 39.8, 39.7, 39.5, 24.0.

HSL432: 5-((6-chloroimidazo[1,2-b]pyridazin-3-yl)ethynyl)-N-(4-((4-methylpiperazin-1-yl)methyl)-3-(trifluoromethyl)phenyl)nicotinamide

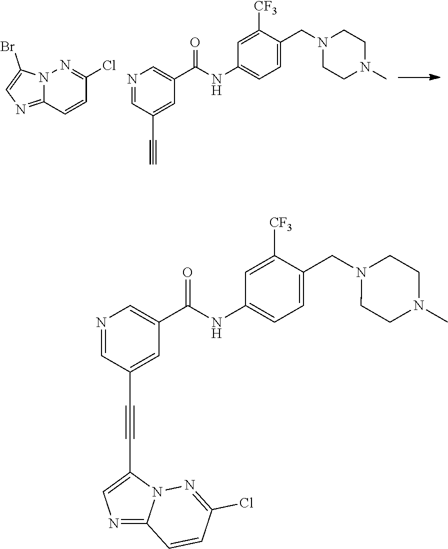

A solution of bromo compound (100 mg, 0.43 mmol, 1 equiv), Pd(PPh₃)₂Cl₂ (10 mol %), CuI (5 mol %) and Triphenylphosphine (10.5 mg, 0.04 mmol, 10 mol %) in triethylamine (1.5 mL, 10.78 mmol, 25.1 equiv) was deoxygenated using argon gas. A deoxygenated solution of alkyne (207.5 mg, 0.52 mmol, 1.2 equiv) in DMF (4 mL) was slowly added over 10 minutes. The reaction was then moved to 50° C. and allowed to run for 15 hrs. The reaction was the cooled to room temperature, diluted with ethyl acetate (150 mL) and washed with water (5×50 mL) and brine (1×50 mL). Combined organic layers were dried over anhydrous sodium sulphate, concentrated in vacuo and purified via column chromatography. Yield: 96.3 mg, 40.4%

¹H NMR (500 MHz, DMSO-d₆) δ 10.81 (s, 1H), 9.12 (d, J=2.2 Hz, 1H), 8.98 (d, J=2.0 Hz, 1H), 8.57 (t, J=2.1 Hz, 1H), 8.34 (d, J=9.5 Hz, 1H), 8.29 (s, 1H), 8.20 (d, J=2.2 Hz, 1H), 8.06-8.01 (m, 1H), 7.71 (d, J=8.5 Hz, 1H), 7.54 (d, J=9.4 Hz, 1H), 3.57 (s, 2H), 2.44 (s, 8H), 2.24 (s, 3H).

HSL442: N-(4-((4-methylpiperazin-1-yl)methyl)-3-(trifluoromethyl)phenyl)-5-(pyrazolo[1,5-a]pyridin-3-ylethynyl)nicotinamide

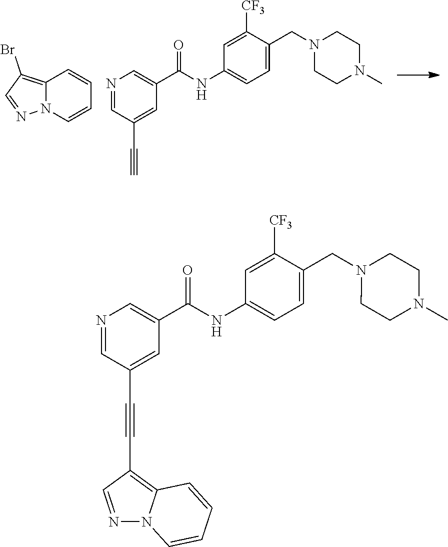

A solution of bromo compound (80 mg, 0.406 mmol, 1 equiv), Pd(PPh₃)₂Cl₂ (10 mol %), CuI (5 mol %) and Triphenylphosphine (10.5 mg, 0.04 mmol, 10 mol %) in triethylamine (1.5 mL, 10.78 mmol, 26.5 equiv) was deoxygenated using argon gas. A deoxygenated solution of alkyne (195.9 mg, 0.487 mmol, 1.2 equiv) in DMf (4 mL) was slowly added over 10 minutes. The reaction was then moved to 50° C. and allowed to run for 15 hrs. The reaction was the cooled to room temperature, diluted with ethyl acetate (150 mL) and washed with water (5×50 mL) and brine (1×50 mL). Combined organic layers were concentrated in vacuo and purified via column chromatography. Yield: 30.2 mg, 14.3%

¹H NMR (500 MHz, DMSO-d₆) δ 10.79 (s, 1H), 9.04 (d, J=2.2 Hz, 1H), 8.95 (d, J=2.0 Hz, 1H), 8.82 (d, J=7.0 Hz, 1H), 8.52 (t, J=2.1 Hz, 1H), 8.36 (s, 1H), 8.21 (d, J=2.2 Hz, 1H), 8.05 (dd, J=8.5, 2.2 Hz, 1H), 7.94 (d, J=8.8 Hz, 1H), 7.72 (d, J=8.5 Hz, 1H), 7.50-7.43 (m, 1H), 7.08 (td, J=6.9, 1.4 Hz, 1H), 3.57 (s, 2H), 2.46-2.30 (m, 8H), 2.23 (s, 3H).

HSL412: N-(4-((4-methylpiperazin-1-yl)methyl)-3-(trifluoromethyl)phenyl)-5-(pyrazolo[1,5-a]pyrimidin-6-ylethynyl)nicotinamide

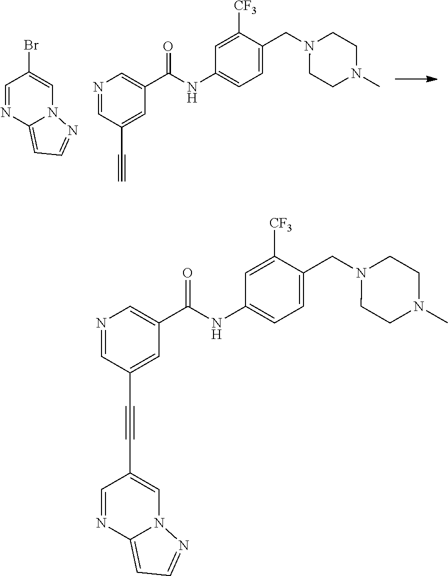

A solution of bromo compound (80 mg, 0.404 mmol, 1 equiv), Pd(PPh₃)₂Cl₂ (10 mol %), CuI (5 mol %) and Triphenylphosphine (10.5 mg, 0.04 mmol, 10 mol %) in triethylamine (1.5 mL, 10.78 mmol, 26.6 equiv) was deoxygenated using argon gas. A deoxygenated solution of alkyne (194.9 mg, 0.484 mmol, 1.2 equiv) in DMF (4 mL) was slowly added over 10 minutes. The reaction was then moved to 50° C. and allowed to run for 15 hrs. The reaction was the cooled to room temperature, diluted with ethyl acetate (150 mL) and washed with water (5×50 mL) and brine (1×50 mL). Combined organic layers were dried over anhydrous sodium sulphate, concentrated in vacuo and purified via column chromatography. Yield: 67.9 mg, 32.2%

¹H NMR (500 MHz, DMSO-d₆) δ 10.86 (s, 1H), 9.58 (d, J=2.0 Hz, 1H), 9.13 (d, J=2.2 Hz, 1H), 8.97 (d, J=2.0 Hz, 1H), 8.71 (d, J=2.1 Hz, 1H), 8.55 (t, J=2.2 Hz, 1H), 8.34 (d, J=2.3 Hz, 1H), 8.21 (d, J=2.2 Hz, 1H), 8.05 (dd, J=8.5, 2.2 Hz, 1H), 7.72 (d, J=8.5 Hz, 1H), 6.84 (d, J=2.2 Hz, 1H), 2.50-2.34 (m, 8H), 2.28 (s, 3H). ¹³C NMR (126 MHz, DMSO-d₆) δ 163.8, 154.4, 151.3, 149.0, 147.1, 147.0, 139.2, 138.3, 138.1, 132.8, 131.9, 130.3, 127.8, 125.8, 124.0, 119.0, 117.8, 104.5, 98.0, 89.2, 87.6, 57.7, 54.7, 52.4, 45.3, 40.5, 40.3, 40.1, 40.0, 39.8, 39.6, 39.5.

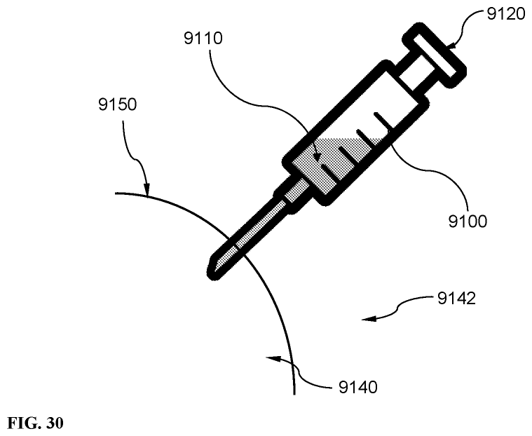

According to some embodiments, the compositions disclosed herein may be delivered to a subject via injection. A composition of one or more of the group compounds selected from group 1 through group ore modified species of the cluster of lncRNA transcripts Mhrt RNAs may be prepared in a dosage form of an injection fluid and be loaded into an injectable device (e.g., a syringe), to inject into a subject's body. FIG. 30 is an illustration of a treatment delivery apparatus (9100) comprising an injectable drug delivery device (9120) and a composition, or combinations of compositions, disclosed herein in the dosage form of an injection fluid (9110). The composition disclosed herein may be delivered through an injection through a wall (9150) of a body part or an organ (9140) of a subject and go into body part or organ (9140) of the subject. In select embodiments, the injectable drug delivery device may stay outside (9142) of body party or organ (9140) of the subject's body.

FIG. 30 represents merely one illustrative embodiment for delivering the disclosed pharmaceutical composition into a subject's body. The delivery instrument may not be limited to a syringe-type device. One of ordinary skill in the art would readily appreciate that any injectable device suitable for delivering the disclose product(s) or agent of a patient's body may be utilized according to aspects of the present invention. For example, the treatment delivery apparatus may be a capsule, polypill, tablet, transdermal patch, or dietary supplement, or a combination of the above, etc. It should also be appreciated that the delivery instrument may be designed for controlled release or delayed release of the disclosed compounds.

All publications, patents and patent applications referred to herein are incorporated by reference in their entirety to the same extent as if each individual publication, patent or patent application was specifically and individually indicated to be incorporated by reference in its entirety.

While the present disclosure has been disclosed with references to certain embodiments, numerous modification, alterations, and changes to the described embodiments are possible without departing from the sphere and scope of the present disclosure, as defined in the appended claims. Accordingly, it is intended that the present disclosure not be limited to the described embodiments, but that it has the full scope defined by the language of the following claims, and equivalents thereof.

### REFERENCES

1. FDA Approves Pronatinib, U.S. Food & Drug Administration https://www.fda.gov/drugs/informationondrugs/approveddrugs/ucm381452.ht m
2. Mingqiang, R. et al. Noel FGFR inhibitor ponatinib suppresses growth of non-small cell lung cancer cells overexpressing FGFR1. Oncol. Rep. 29, 2181-2190 (2013).
3. De Falco, V. et al. Ponatinib (AP24534) is a novel potent inhibitor of oncogenic RET mutants associated with thyroid cancer. J. Clin. Endocrinol. Metab. 98, E811-E819 (2013).
4. Gibbons, D. L., Pricl, S. Kantargian, H., Cortes, J., & Quintas-Cardama, A. The rise and fall of gatekeeper mutations? The BCR-ABL T315I paradigm. Cancer. 118, 293-299 (2011).
5. NCT02428543 Ponatinib for FLT3-ITD acute myelogenous leukemia (PONATINIB-AML), NIH U.S. National Library of Medicine https://clinicaltrials.gov/ct2/show/NCT02428543
6. NCT02265341 Ponatinib hydrochloride in treating patients with advanced biliary cancer with fgfr2 fusions, NIH U.S. National Library of Medicine https://clinicaltrials.gov/ct2/NCT02265341
7. NCT01813734 ponatinib in advanced NSCLC w/ RET translocation, NIH U.S. National Library of Medicine https://clinicaltrials.gov/ct2/show/NCT01813734
8. Aghel, N., Delgado, D. H., & Lipton, J. H. cardiovascular toxicities of BCR-ABL tyrosine kinase inhibitors in chronic myeloid leukemia: preventive strategies and caridovasuclar surveillance. Vasc. Health. Risk. Manag. 13, 293-303 (2017).
9. Pasvolsky, O. et al. Tyrosine kinase inhibitor associated vascular toxicity in chronic myeloid leukemia. Cardio-Oncology. 1; 10.1186/s40959-015-0008-5 (2015).
10. Banavath, H. N., Sharma, O. P., Kumar, M. S., & Baskaran, R. Identification of novel tyrosine kinase inhibitors for drug resistant T315I muatant BCR-ABL: a virtual screening and molecular dynamics simulations study. Sci. Rep. 4, 6948; DOI: 10.1038/srep06948 (2014).
11. Moslehi, J. J., & Meininger, M. Tyrosine kinase inhibitor-associated cardiovascular toxicity in chronic myeloid leukemia. J. Clin. Oncol., 35, 4210-7218 (2015).
12. Hou J et al., Targeting Mnks for Cancer Therapy. Oncotarget, 118-131 (2012).
13. Log P was calculated using the Swiss Institute of Bioinformatics SwissADME online software.
14. Kuppuswamy, D. et al. Association of tyrosine-phosphorylated c-Src with the cytoskeleton of hypertrophying myocardium. J. Biol. Chem. 272, 4500-4501 (1997).
15. Schlichter, L. C. et al. Regulation of hERG and hEAG channels by Src and by SHP-1 tyrosine phosphatase via an ITIM region in the cyclic nucleotide binding domain. Plos. One. 9, e90024; doi: 10.1371/journal.pone.0090024 (2014).
16. Golden, A., Nemeth S. P., & Brugge, J. S. Blood platelets express high levels of the pp60c-src-specific tyrosine kinase activity. PNAS. 83, 852-856 (1986).
17. Shattil, S. J., Kim, C., & Ginsberg, M. H. The final steps of integrin activation: the end game. Nat. Rev. Mol. Cell. Biol. 11, 288-300 (2010).
18. Hierro, H., Roden, J., & Tabernero, J. Fibroblast Growth Factor (FGF) Receptor/FGF inhibitors: novel targets and strategies for optimization of response of solid tumors. Semin. Oncol. 42, 801-819 (2015).
19. Yanochko, G. M. et al. Pan-FGFR inhibition leads to blockade of FGF23 signaling, soft tissue mineralization, and cardiovascular dysfunction. Toxicol. Sci. 135, 451-464 (2013).
20. Daver, N. et al. Secondary mutations as mediators of resistance to targeted therapy in leukemia. Blood. 125, 3236-3245 (2015).
21. Levis, M. FLT3 mutations in acute myeloid leukemia: what is the best approach in 2013? Hematology. Am. Soc. Hematol. Educ. Program. 2013, 220-226 (2013).

1. A compound represented by a compound of Formula (I): 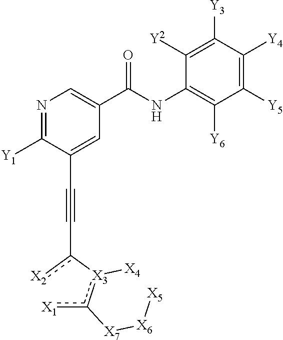
Wherein Y1-Y6==amide; O-alkyl such as OMe, OEt, OPr, OBu, OiPr, OCF₂, OCF₃; NH₂, NH-alkyl, such as NHMe, NHEt; N-(alkyl)₂ or N-(heteroalkyl)₂, such as NMe₂, morpholino, piperazine; CN, Cl, Br, iPr, Et, cyclopropyl, butyl, CF₃, CHF₂, CH₂-piperazine analog, CH₂-morpholine analog, CH₂-piperidine analog, CH₂-pyrrolidine analog, CH₂-azetidine analog, etc. and X1-X7=CH, CY or N.

2. The compound of claim 1, wherein said compound is a compound of the following formula:

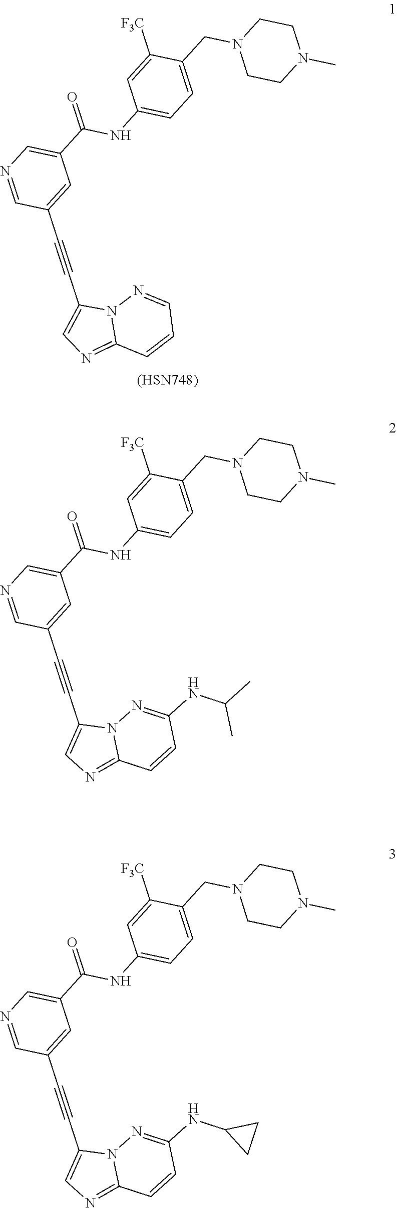

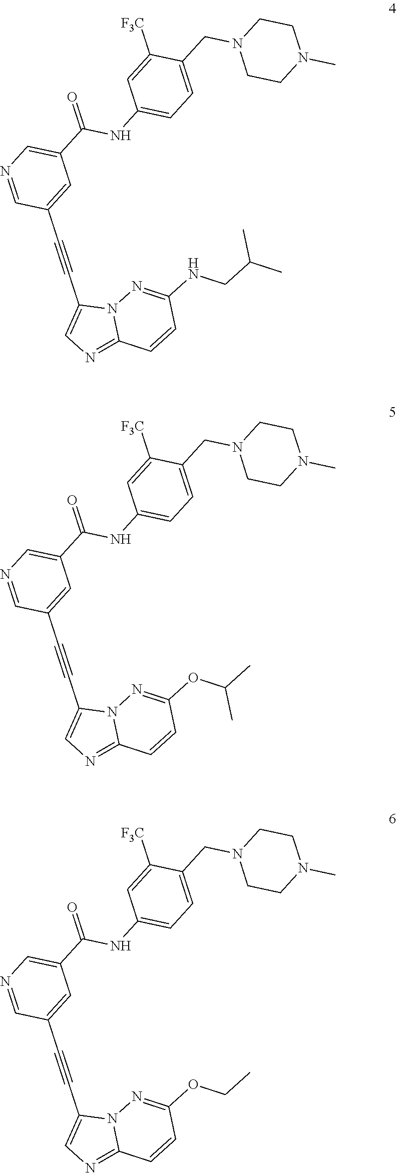 [SMILES: CC(C)CNc1ccc2ncc(C#Cc3cncc(C(=O)Nc4ccc(CN5CCN(C)CC5)c(C(F)(F)F)c4)c3)n2n1 ; CC(C)Oc1ccc2ncc(C#Cc3cncc(C(=O)Nc4ccc(CN5CCN(C)CC5)c(C(F)(F)F)c4)c3)n2n1 ; CCOc1ccc2ncc(C#Cc3cncc(C(=O)Nc4ccc(CN5CCN(C)CC5)c(C(F)(F)F)c4)c3)n2n1]

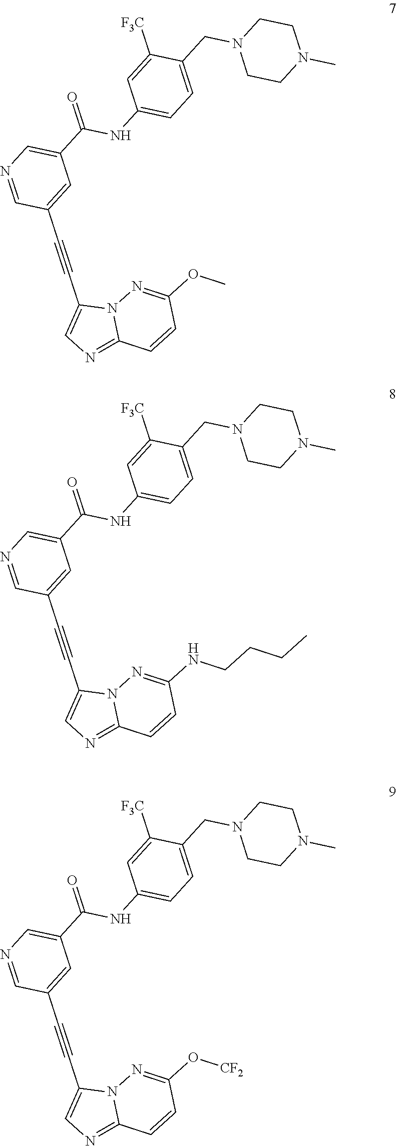

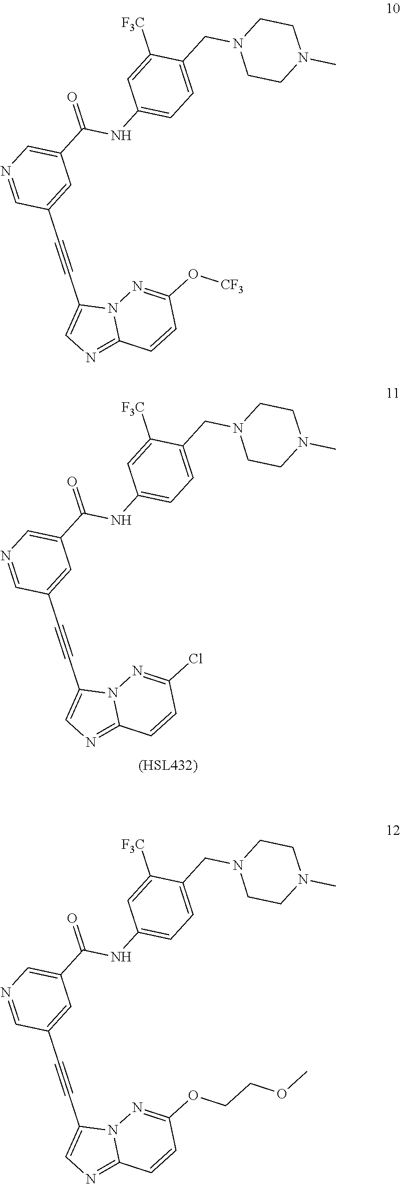 [SMILES: CN1CCN(Cc2ccc(NC(=O)c3cncc(C#Cc4cnc5ccc(OC(F)(F)F)nn45)c3)cc2C(F)(F)F)CC1 ; CN1CCN(Cc2ccc(NC(=O)c3cncc(C#Cc4cnc5ccc(Cl)nn45)c3)cc2C(F)(F)F)CC1 ; COCCOc1ccc2ncc(C#Cc3cncc(C(=O)Nc4ccc(CN5CCN(C)CC5)c(C(F)(F)F)c4)c3)n2n1]

3. The compound of claim 1, wherein said compound is a compound of the following formula:

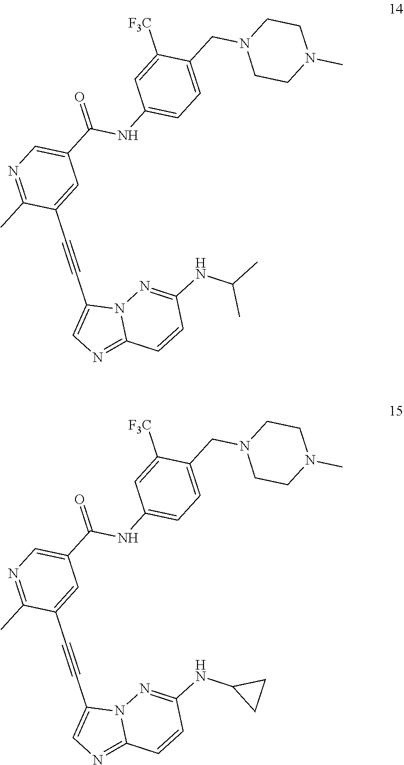

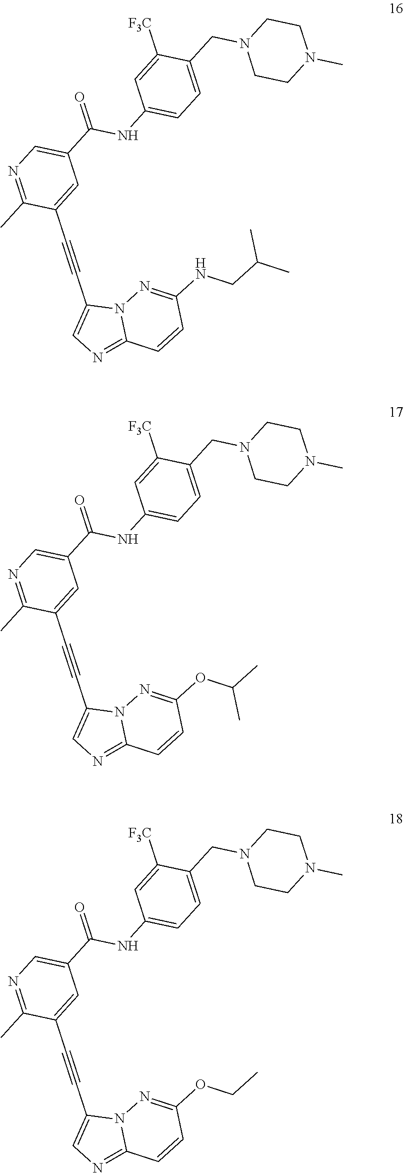

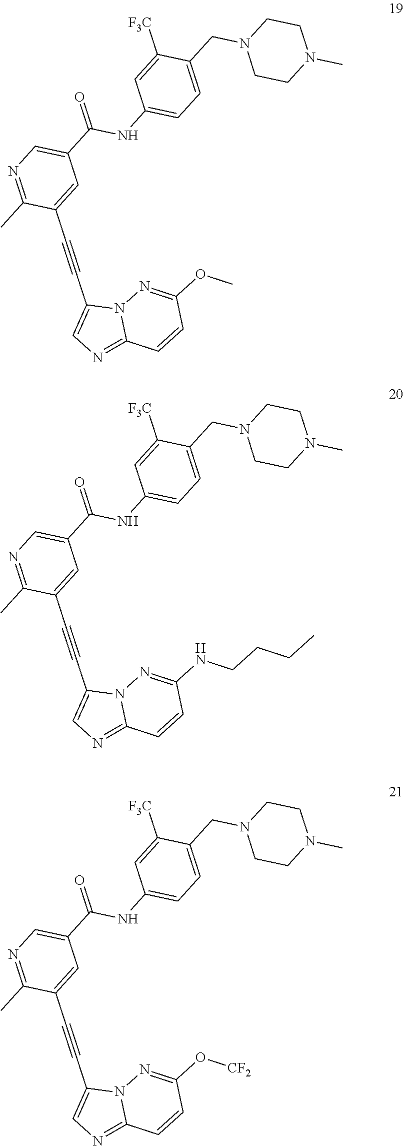

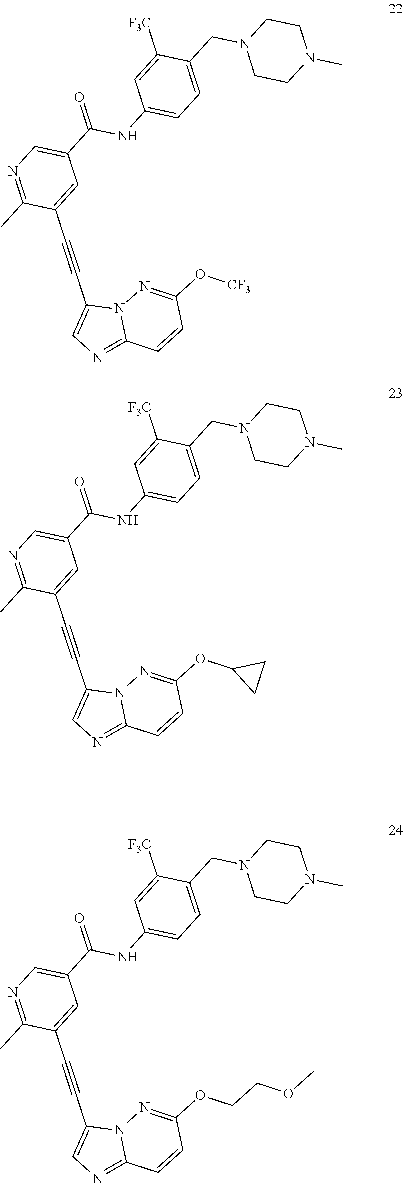

4. The compound of claim 1, wherein said compound is a compound of the following formula:

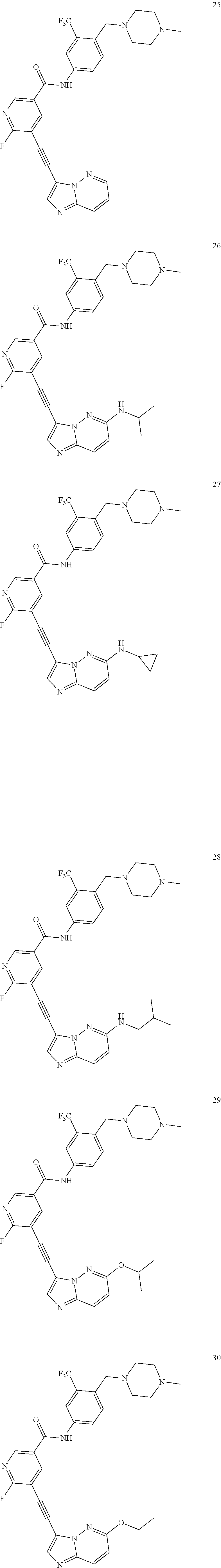 [SMILES: CN1CCN(Cc2ccc(NC(=O)c3cnc(F)c(C#Cc4cnc5cccnn45)c3)cc2C(F)(F)F)CC1 ; CC(C)Nc1ccc2ncc(C#Cc3cc(C(=O)Nc4ccc(CN5CCN(C)CC5)c(C(F)(F)F)c4)cnc3F)n2n1 ; CN1CCN(Cc2ccc(NC(=O)c3cnc(F)c(C#Cc4cnc5ccc(NC6CC6)nn45)c3)cc2C(F)(F)F)CC1 ; CC(C)CNc1ccc2ncc(C#Cc3cc(C(=O)Nc4ccc(CN5CCN(C)CC5)c(C(F)(F)F)c4)cnc3F)n2n1 ; CC(C)Oc1ccc2ncc(C#Cc3cc(C(=O)Nc4ccc(CN5CCN(C)CC5)c(C(F)(F)F)c4)cnc3F)n2n1 ; CCOc1ccc2ncc(C#Cc3cc(C(=O)Nc4ccc(CN5CCN(C)CC5)c(C(F)(F)F)c4)cnc3F)n2n1]

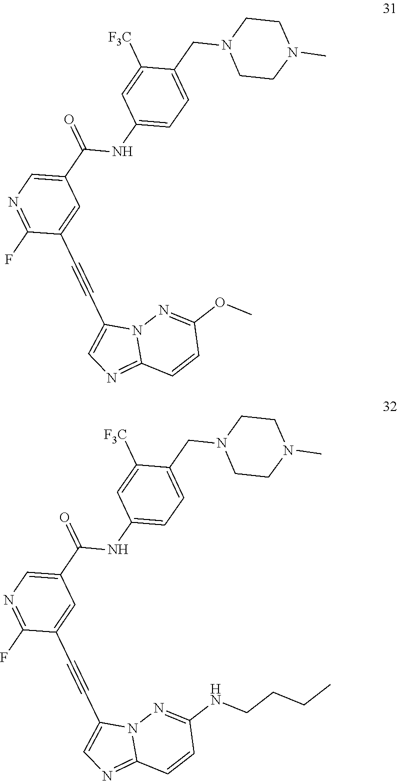 [SMILES: COc1ccc2ncc(C#Cc3cc(C(=O)Nc4ccc(CN5CCN(C)CC5)c(C(F)(F)F)c4)cnc3F)n2n1 ; CCCCNc1ccc2ncc(C#Cc3cc(C(=O)Nc4ccc(CN5CCN(C)CC5)c(C(F)(F)F)c4)cnc3F)n2n1]

5. The compound of claim 1, wherein said compound is a compound of the following formula:

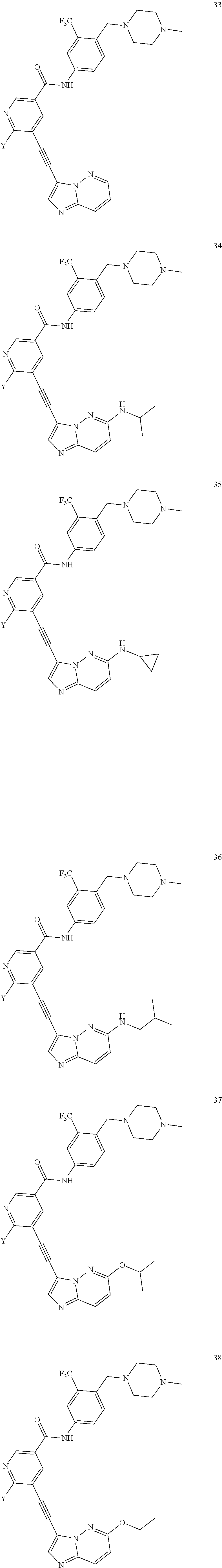

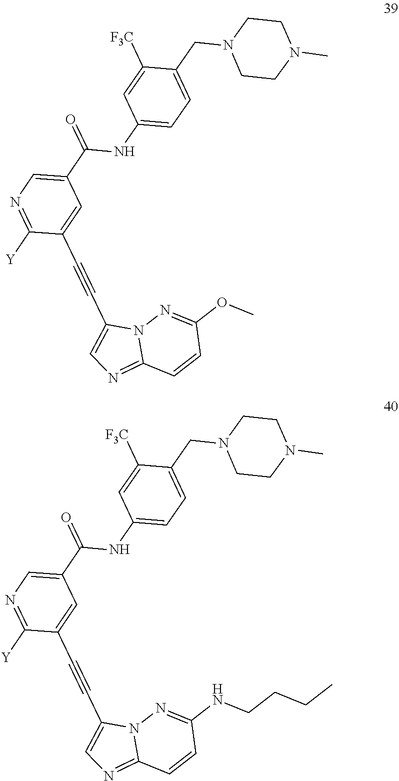

6. The compound of claim 1, wherein said compound is a compound of the following formula:

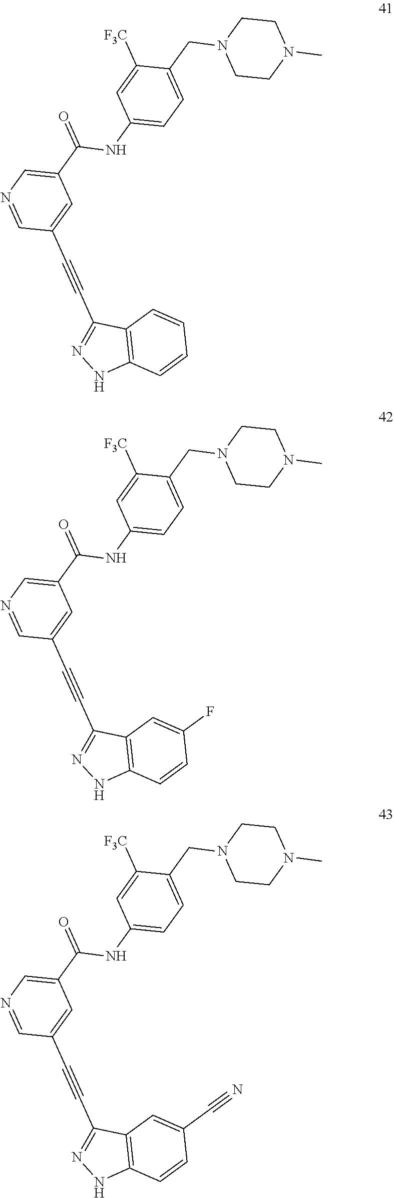 [SMILES: CN1CCN(Cc2ccc(NC(=O)c3cncc(C#Cc4n[nH]c5ccccc45)c3)cc2C(F)(F)F)CC1 ; CN1CCN(Cc2ccc(NC(=O)c3cncc(C#Cc4n[nH]c5ccc(F)cc45)c3)cc2C(F)(F)F)CC1 ; CN1CCN(Cc2ccc(NC(=O)c3cncc(C#Cc4n[nH]c5ccc(C#N)cc45)c3)cc2C(F)(F)F)CC1]

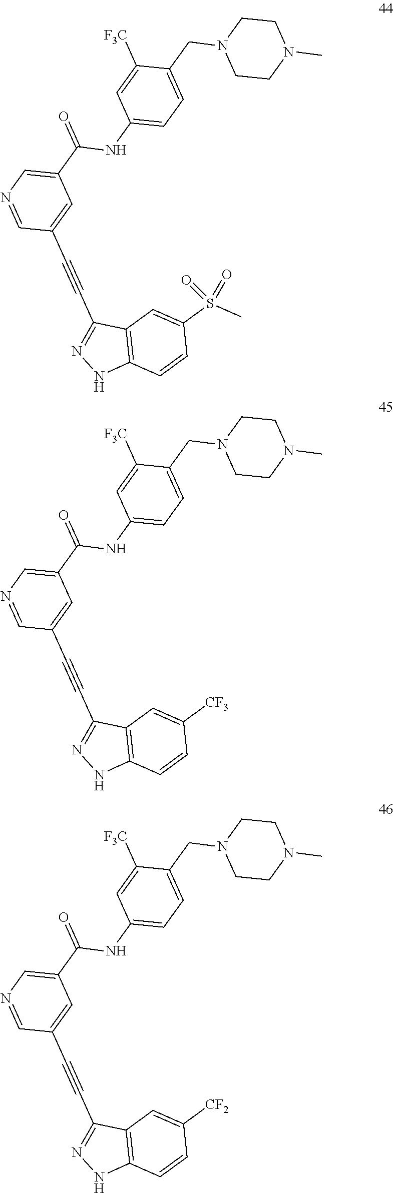

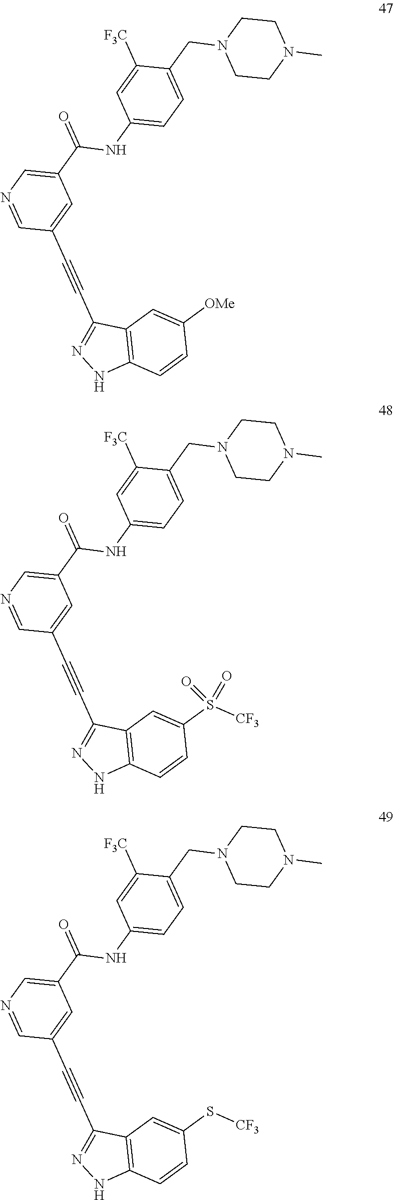 [SMILES: COc1ccc2[nH]nc(C#Cc3cncc(C(=O)Nc4ccc(CN5CCN(C)CC5)c(C(F)(F)F)c4)c3)c2c1 ; CN1CCN(Cc2ccc(NC(=O)c3cncc(C#Cc4n[nH]c5ccc(S(=O)(=O)C(F)(F)F)cc45)c3)cc2C(F)(F)F)CC1 ; CN1CCN(Cc2ccc(NC(=O)c3cncc(C#Cc4n[nH]c5ccc(SC(F)(F)F)cc45)c3)cc2C(F)(F)F)CC1]

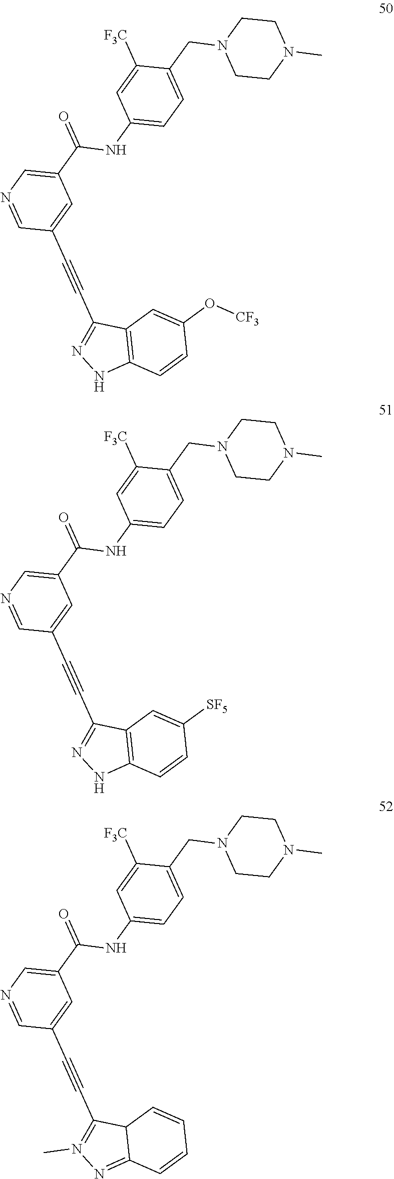

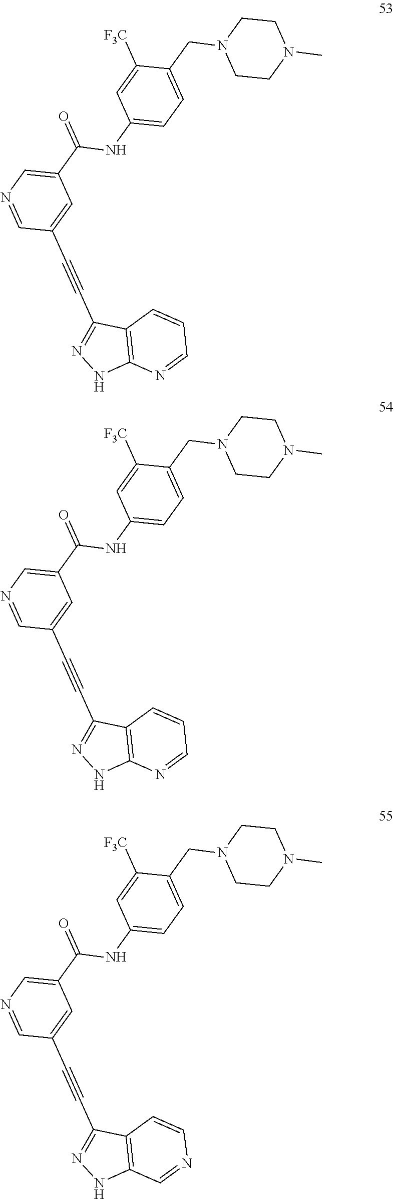 [SMILES: CN1CCN(Cc2ccc(NC(=O)c3cncc(C#Cc4n[nH]c5ncccc45)c3)cc2C(F)(F)F)CC1 ; CN1CCN(Cc2ccc(NC(=O)c3cncc(C#Cc4n[nH]c5ncccc45)c3)cc2C(F)(F)F)CC1 ; CN1CCN(Cc2ccc(NC(=O)c3cncc(C#Cc4n[nH]c5cnccc45)c3)cc2C(F)(F)F)CC1]

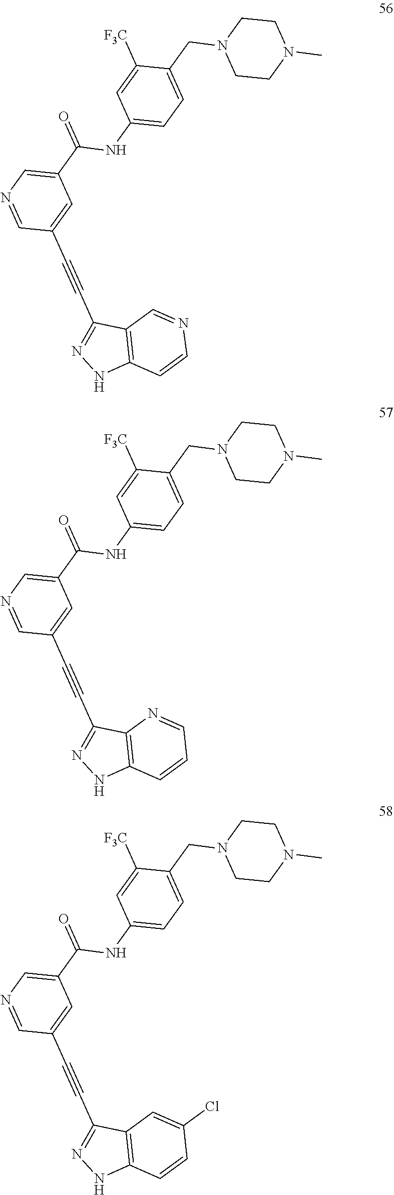 [SMILES: CN1CCN(Cc2ccc(NC(=O)c3cncc(C#Cc4n[nH]c5ccncc45)c3)cc2C(F)(F)F)CC1 ; CN1CCN(Cc2ccc(NC(=O)c3cncc(C#Cc4n[nH]c5cccnc45)c3)cc2C(F)(F)F)CC1 ; CN1CCN(Cc2ccc(NC(=O)c3cncc(C#Cc4n[nH]c5ccc(Cl)cc45)c3)cc2C(F)(F)F)CC1]

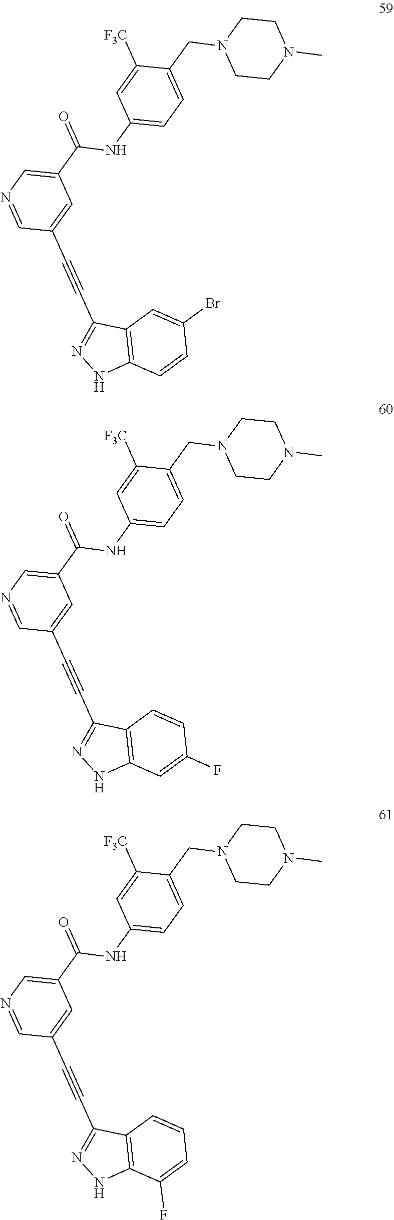 [SMILES: CN1CCN(Cc2ccc(NC(=O)c3cncc(C#Cc4n[nH]c5ccc(Br)cc45)c3)cc2C(F)(F)F)CC1 ; CN1CCN(Cc2ccc(NC(=O)c3cncc(C#Cc4n[nH]c5cc(F)ccc45)c3)cc2C(F)(F)F)CC1 ; CN1CCN(Cc2ccc(NC(=O)c3cncc(C#Cc4n[nH]c5c(F)cccc45)c3)cc2C(F)(F)F)CC1]

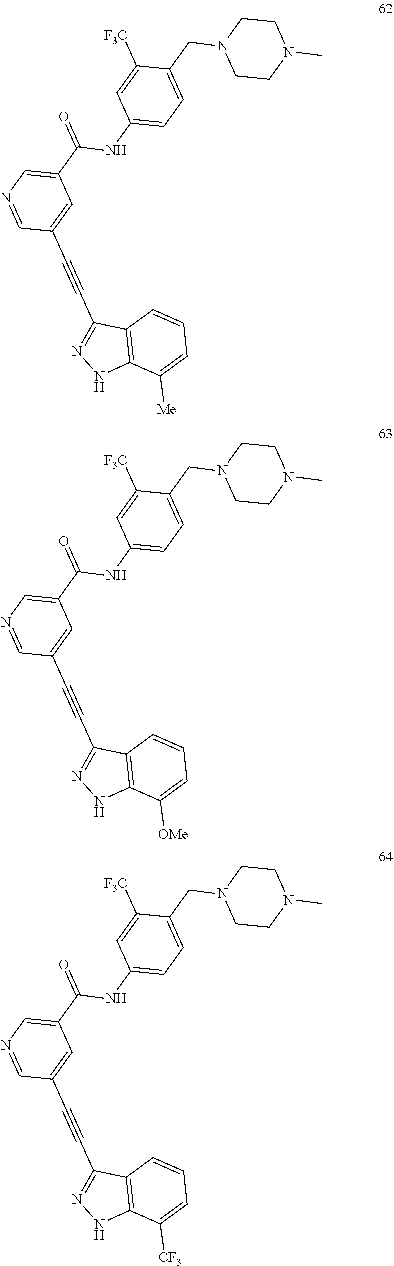 [SMILES: Cc1cccc2c(C#Cc3cncc(C(=O)Nc4ccc(CN5CCN(C)CC5)c(C(F)(F)F)c4)c3)n[nH]c12 ; COc1cccc2c(C#Cc3cncc(C(=O)Nc4ccc(CN5CCN(C)CC5)c(C(F)(F)F)c4)c3)n[nH]c12 ; CN1CCN(Cc2ccc(NC(=O)c3cncc(C#Cc4n[nH]c5c(C(F)(F)F)cccc45)c3)cc2C(F)(F)F)CC1]

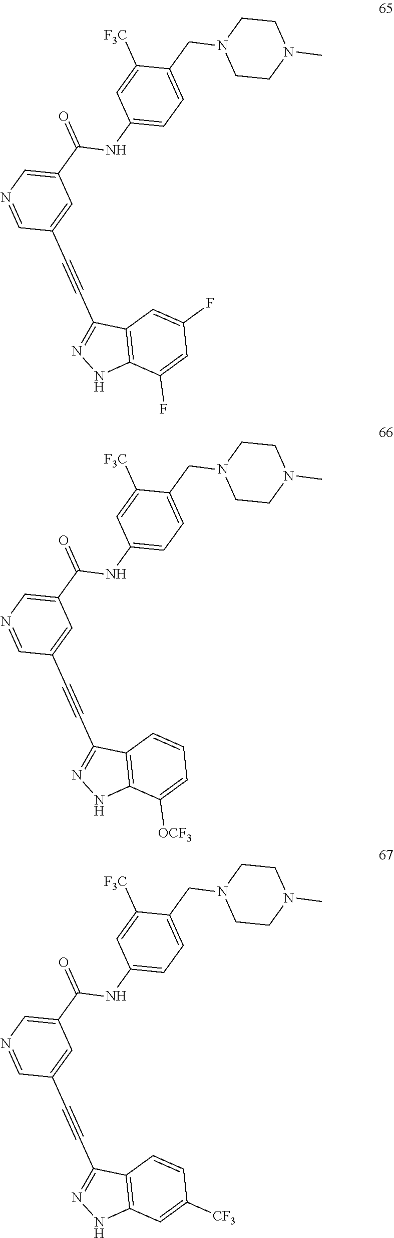 [SMILES: CN1CCN(Cc2ccc(NC(=O)c3cncc(C#Cc4n[nH]c5c(F)cc(F)cc45)c3)cc2C(F)(F)F)CC1 ; CN1CCN(Cc2ccc(NC(=O)c3cncc(C#Cc4n[nH]c5c(OC(F)(F)F)cccc45)c3)cc2C(F)(F)F)CC1 ; CN1CCN(Cc2ccc(NC(=O)c3cncc(C#Cc4n[nH]c5cc(C(F)(F)F)ccc45)c3)cc2C(F)(F)F)CC1]

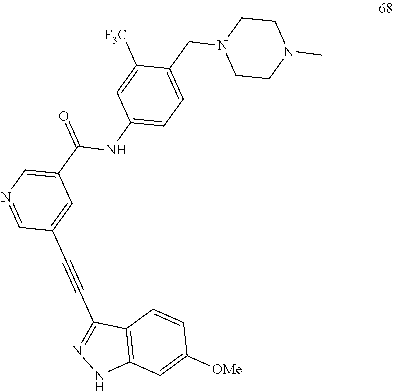 [SMILES: COc1ccc2c(C#Cc3cncc(C(=O)Nc4ccc(CN5CCN(C)CC5)c(C(F)(F)F)c4)c3)n[nH]c2c1]

7. The compound of claim 1, wherein said compound is a compound of the following formula:

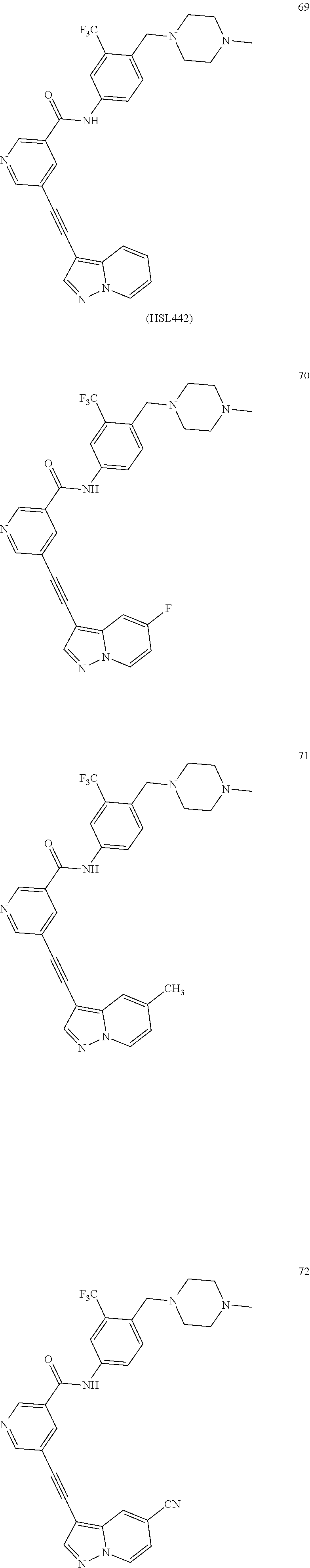 [SMILES: CN1CCN(Cc2ccc(NC(=O)c3cncc(C#Cc4cnn5ccccc45)c3)cc2C(F)(F)F)CC1 ; CN1CCN(Cc2ccc(NC(=O)c3cncc(C#Cc4cnn5ccc(F)cc45)c3)cc2C(F)(F)F)CC1 ; Cc1ccn2ncc(C#Cc3cncc(C(=O)Nc4ccc(CN5CCN(C)CC5)c(C(F)(F)F)c4)c3)c2c1 ; CN1CCN(Cc2ccc(NC(=O)c3cncc(C#Cc4cnn5ccc(C#N)cc45)c3)cc2C(F)(F)F)CC1]

8. The compound of claim 1, wherein said compound is a compound of the following formula:

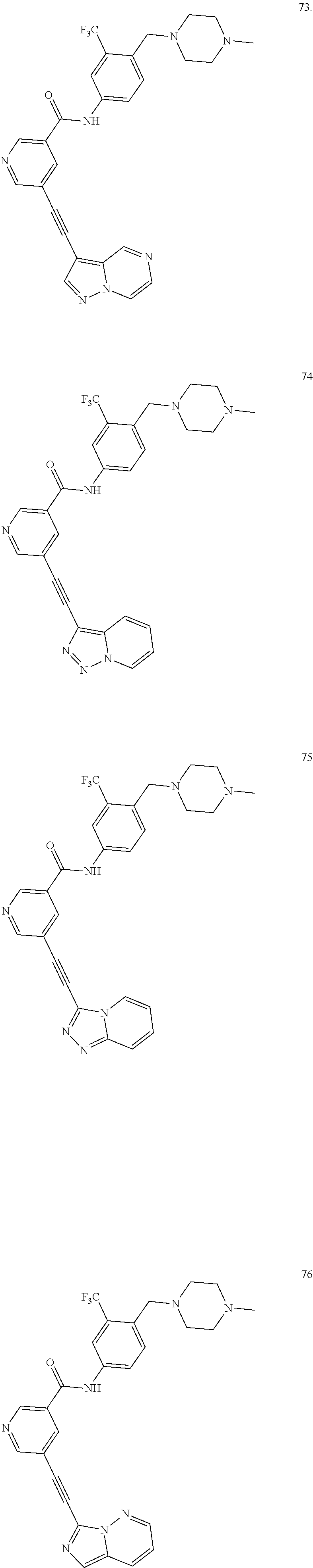 [SMILES: CN1CCN(Cc2ccc(NC(=O)c3cncc(C#Cc4cnn5ccncc45)c3)cc2C(F)(F)F)CC1 ; CN1CCN(Cc2ccc(NC(=O)c3cncc(C#Cc4nnn5ccccc45)c3)cc2C(F)(F)F)CC1 ; CN1CCN(Cc2ccc(NC(=O)c3cncc(C#Cc4nnc5ccccn45)c3)cc2C(F)(F)F)CC1 ; CN1CCN(Cc2ccc(NC(=O)c3cncc(C#Cc4ncc5cccnn45)c3)cc2C(F)(F)F)CC1]

9. The compound of claim 1, wherein said compound is a compound of the following formula:

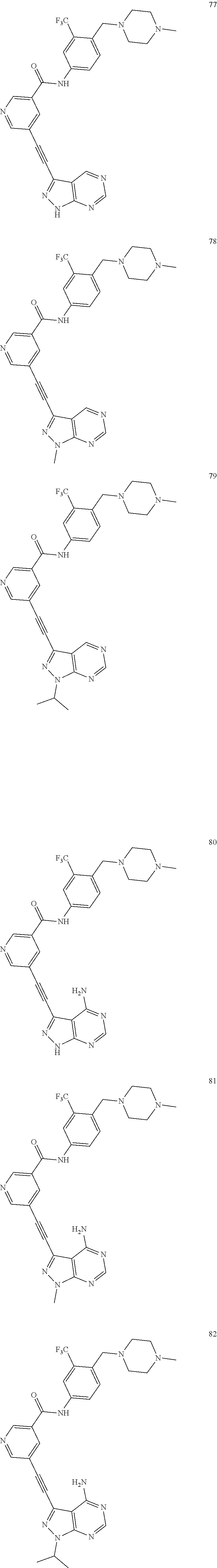 [SMILES: CN1CCN(Cc2ccc(NC(=O)c3cncc(C#Cc4n[nH]c5ncncc45)c3)cc2C(F)(F)F)CC1 ; CN1CCN(Cc2ccc(NC(=O)c3cncc(C#Cc4nn(C)c5ncncc45)c3)cc2C(F)(F)F)CC1 ; CC(C)n1nc(C#Cc2cncc(C(=O)Nc3ccc(CN4CCN(C)CC4)c(C(F)(F)F)c3)c2)c2cncnc21 ; CN1CCN(Cc2ccc(NC(=O)c3cncc(C#Cc4n[nH]c5ncnc(N)c45)c3)cc2C(F)(F)F)CC1 ; CN1CCN(Cc2ccc(NC(=O)c3cncc(C#Cc4nn(C)c5ncnc(N)c45)c3)cc2C(F)(F)F)CC1 ; CC(C)n1nc(C#Cc2cncc(C(=O)Nc3ccc(CN4CCN(C)CC4)c(C(F)(F)F)c3)c2)c2c(N)ncnc21]

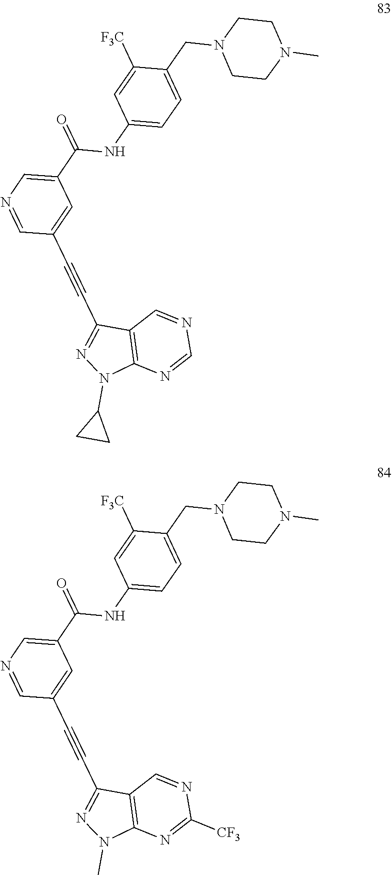 [SMILES: CN1CCN(Cc2ccc(NC(=O)c3cncc(C#Cc4nn(C5CC5)c5ncncc45)c3)cc2C(F)(F)F)CC1 ; CN1CCN(Cc2ccc(NC(=O)c3cncc(C#Cc4nn(C)c5nc(C(F)(F)F)ncc45)c3)cc2C(F)(F)F)CC1]

10. The compound of claim 1, wherein said compound is a compound of the following formula:

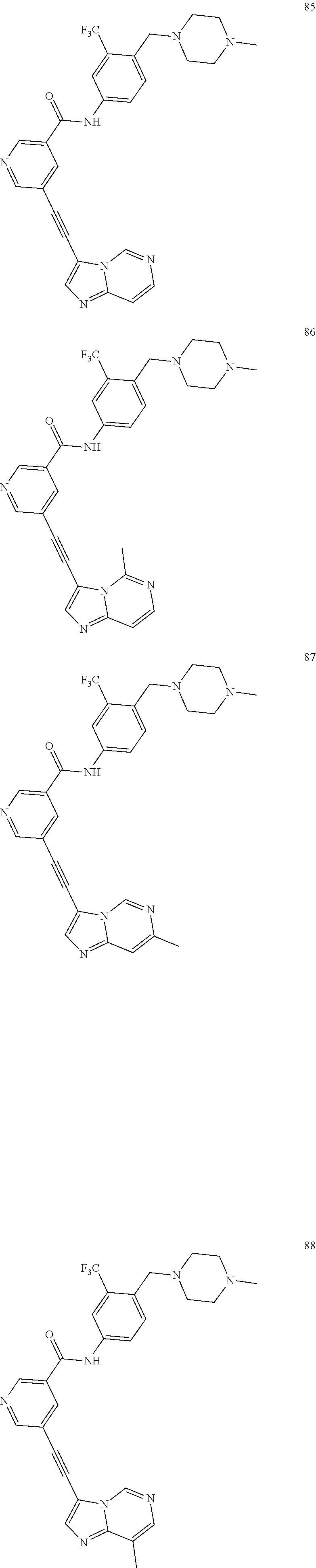 [SMILES: CN1CCN(Cc2ccc(NC(=O)c3cncc(C#Cc4cnc5ccncn45)c3)cc2C(F)(F)F)CC1 ; Cc1nccc2ncc(C#Cc3cncc(C(=O)Nc4ccc(CN5CCN(C)CC5)c(C(F)(F)F)c4)c3)n12 ; Cc1cc2ncc(C#Cc3cncc(C(=O)Nc4ccc(CN5CCN(C)CC5)c(C(F)(F)F)c4)c3)n2cn1 ; Cc1cncn2c(C#Cc3cncc(C(=O)Nc4ccc(CN5CCN(C)CC5)c(C(F)(F)F)c4)c3)cnc12]

11. The compound of claim 1, wherein said compound is a compound of the following formula:

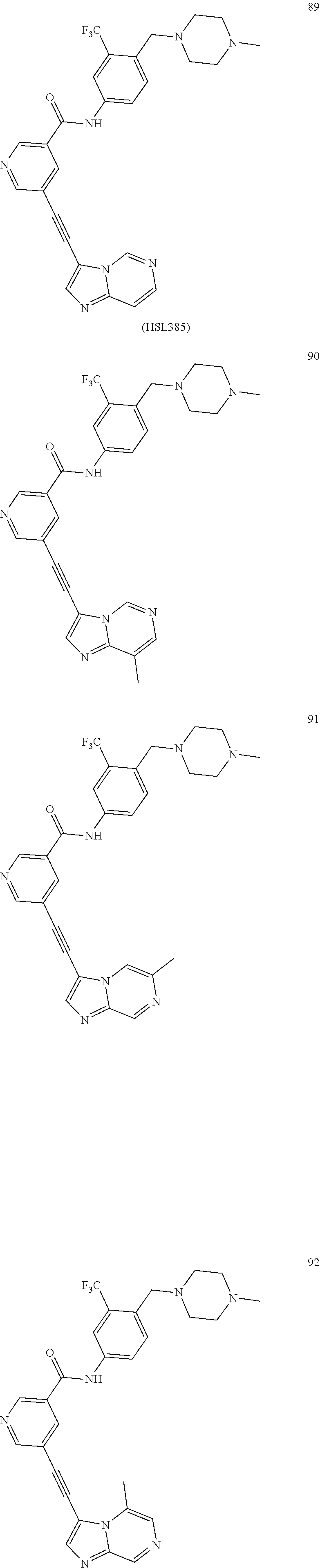 [SMILES: CN1CCN(Cc2ccc(NC(=O)c3cncc(C#Cc4cnc5ccncn45)c3)cc2C(F)(F)F)CC1 ; Cc1cncn2c(C#Cc3cncc(C(=O)Nc4ccc(CN5CCN(C)CC5)c(C(F)(F)F)c4)c3)cnc12 ; Cc1cn2c(C#Cc3cncc(C(=O)Nc4ccc(CN5CCN(C)CC5)c(C(F)(F)F)c4)c3)cnc2cn1 ; Cc1cncc2ncc(C#Cc3cncc(C(=O)Nc4ccc(CN5CCN(C)CC5)c(C(F)(F)F)c4)c3)n12]

12. The compound of claim 1, wherein said compound is a compound of the following formula:

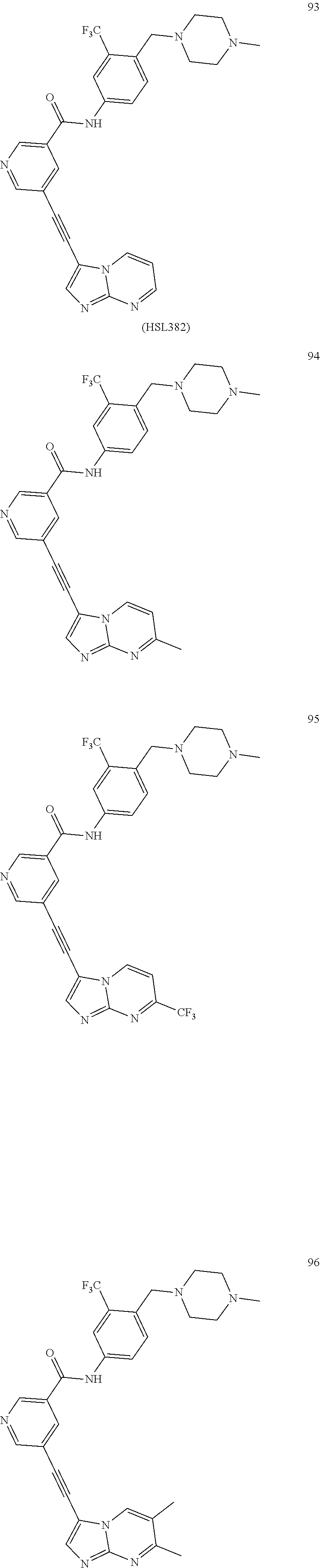 [SMILES: CN1CCN(Cc2ccc(NC(=O)c3cncc(C#Cc4cnc5ncccn45)c3)cc2C(F)(F)F)CC1 ; Cc1ccn2c(C#Cc3cncc(C(=O)Nc4ccc(CN5CCN(C)CC5)c(C(F)(F)F)c4)c3)cnc2n1 ; CN1CCN(Cc2ccc(NC(=O)c3cncc(C#Cc4cnc5nc(C(F)(F)F)ccn45)c3)cc2C(F)(F)F)CC1 ; Cc1cn2c(C#Cc3cncc(C(=O)Nc4ccc(CN5CCN(C)CC5)c(C(F)(F)F)c4)c3)cnc2nc1C]

13. The compound of claim 1, wherein said compound is a compound of the following formula:

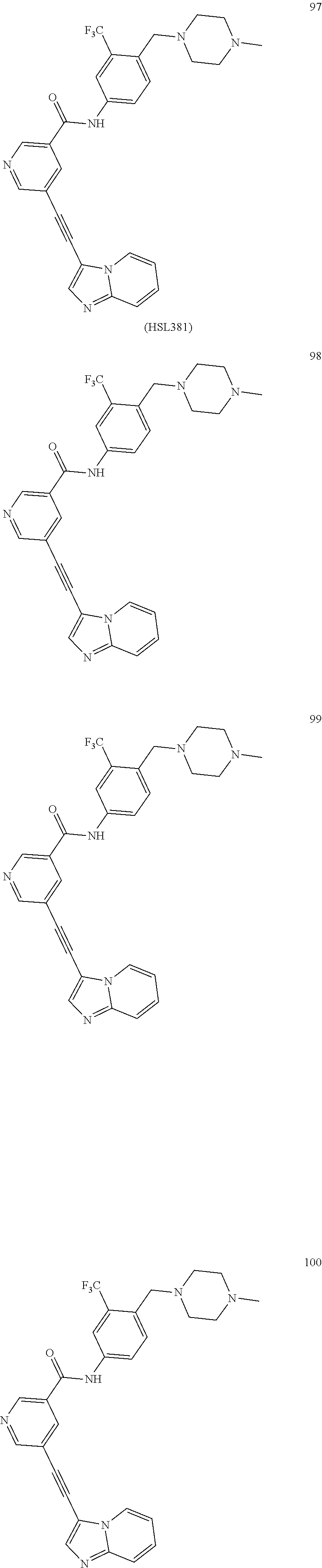 [SMILES: CN1CCN(Cc2ccc(NC(=O)c3cncc(C#Cc4cnc5ccccn45)c3)cc2C(F)(F)F)CC1 ; CN1CCN(Cc2ccc(NC(=O)c3cncc(C#Cc4cnc5ccccn45)c3)cc2C(F)(F)F)CC1 ; CN1CCN(Cc2ccc(NC(=O)c3cncc(C#Cc4cnc5ccccn45)c3)cc2C(F)(F)F)CC1 ; CN1CCN(Cc2ccc(NC(=O)c3cncc(C#Cc4cnc5ccccn45)c3)cc2C(F)(F)F)CC1]

14. The compound of claim 1, wherein said compound is a compound of the following formula:

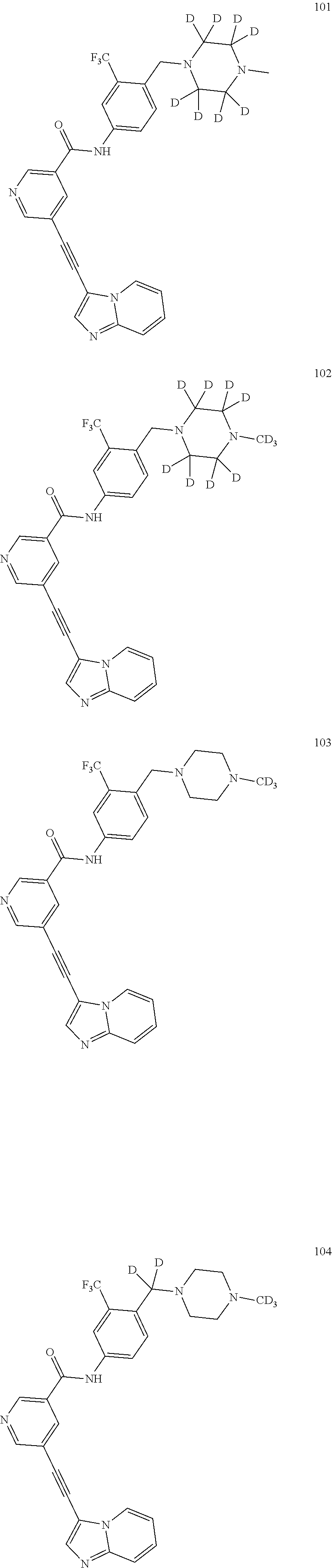 [SMILES: [2H]C1([2H])N(C)C([2H])([2H])C([2H])([2H])N(Cc2ccc(NC(=O)c3cncc(C#Cc4cnc5ccccn45)c3)cc2C(F)(F)F)C1([2H])[2H] ; [2H]C([2H])([2H])N1C([2H])([2H])C([2H])([2H])N(Cc2ccc(NC(=O)c3cncc(C#Cc4cnc5ccccn45)c3)cc2C(F)(F)F)C([2H])([2H])C1([2H])[2H] ; [2H]C([2H])([2H])N1CCN(Cc2ccc(NC(=O)c3cncc(C#Cc4cnc5ccccn45)c3)cc2C(F)(F)F)CC1 ; [2H]C([2H])([2H])N1CCN(C([2H])([2H])c2ccc(NC(=O)c3cncc(C#Cc4cnc5ccccn45)c3)cc2C(F)(F)F)CC1]

15. The compound of claim 1, wherein said compound is a compound of the following formula:

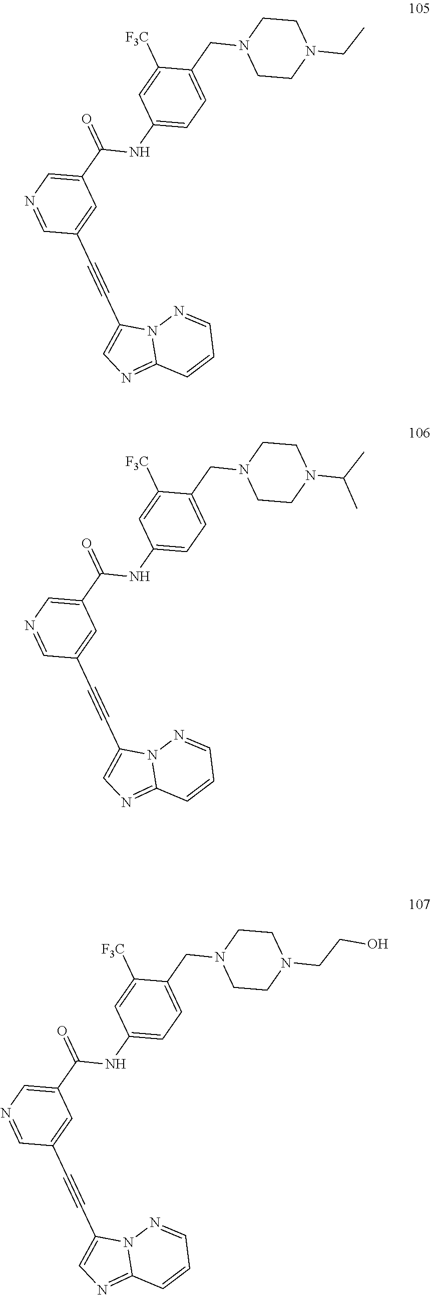 [SMILES: CCN1CCN(Cc2ccc(NC(=O)c3cncc(C#Cc4cnc5cccnn45)c3)cc2C(F)(F)F)CC1 ; CC(C)N1CCN(Cc2ccc(NC(=O)c3cncc(C#Cc4cnc5cccnn45)c3)cc2C(F)(F)F)CC1 ; O=C(Nc1ccc(CN2CCN(CCO)CC2)c(C(F)(F)F)c1)c1cncc(C#Cc2cnc3cccnn23)c1]

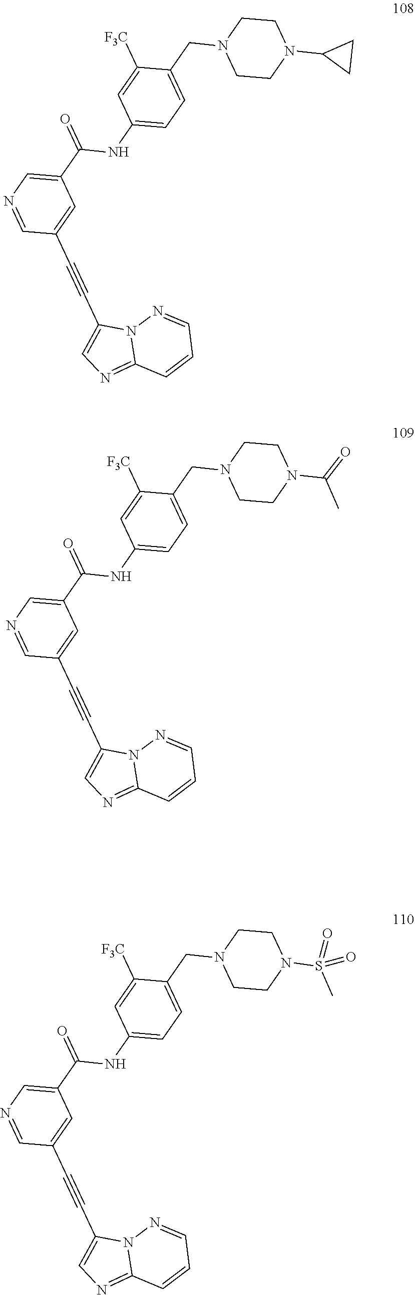 [SMILES: O=C(Nc1ccc(CN2CCN(C3CC3)CC2)c(C(F)(F)F)c1)c1cncc(C#Cc2cnc3cccnn23)c1 ; CC(=O)N1CCN(Cc2ccc(NC(=O)c3cncc(C#Cc4cnc5cccnn45)c3)cc2C(F)(F)F)CC1 ; CS(=O)(=O)N1CCN(Cc2ccc(NC(=O)c3cncc(C#Cc4cnc5cccnn45)c3)cc2C(F)(F)F)CC1]

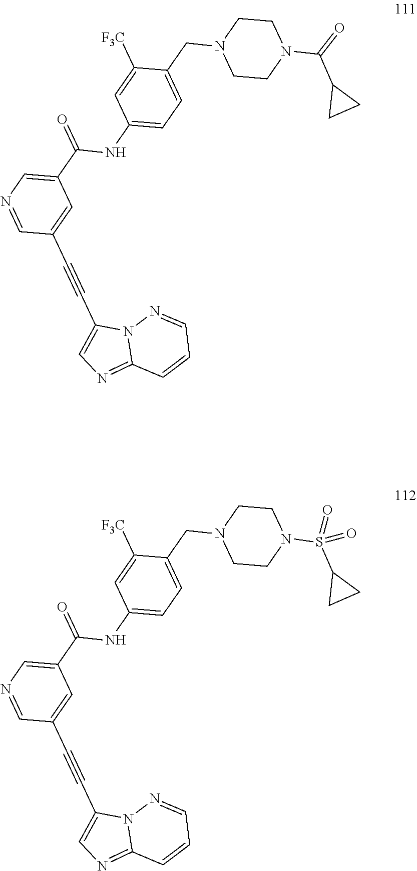 [SMILES: O=C(Nc1ccc(CN2CCN(C(=O)C3CC3)CC2)c(C(F)(F)F)c1)c1cncc(C#Cc2cnc3cccnn23)c1 ; O=C(Nc1ccc(CN2CCN(S(=O)(=O)C3CC3)CC2)c(C(F)(F)F)c1)c1cncc(C#Cc2cnc3cccnn23)c1]

16. The compound of claim 1, wherein said compound is a compound of the following formula:

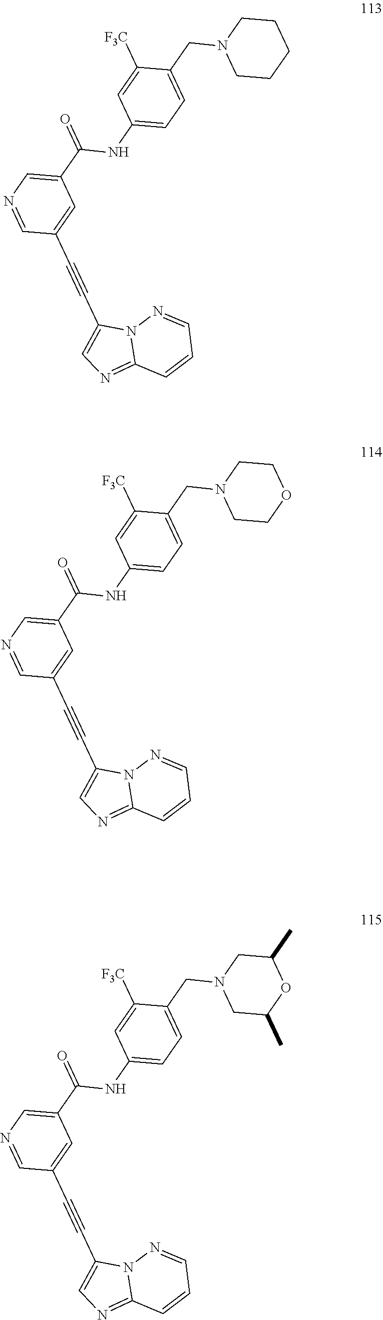 [SMILES: O=C(Nc1ccc(CN2CCCCC2)c(C(F)(F)F)c1)c1cncc(C#Cc2cnc3cccnn23)c1 ; O=C(Nc1ccc(CN2CCOCC2)c(C(F)(F)F)c1)c1cncc(C#Cc2cnc3cccnn23)c1 ; C[C@@H]1CN(Cc2ccc(NC(=O)c3cncc(C#Cc4cnc5cccnn45)c3)cc2C(F)(F)F)C[C@H](C)O1]

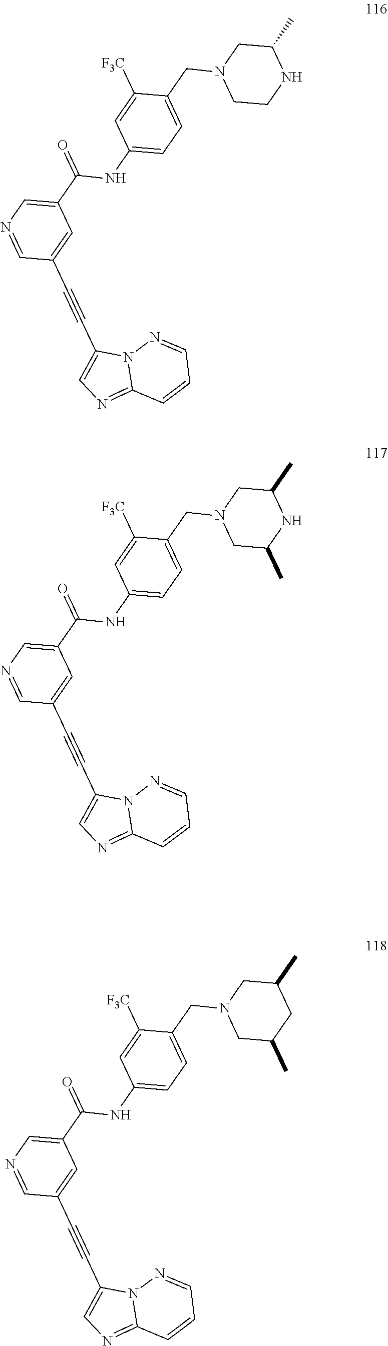 [SMILES: C[C@H]1CN(Cc2ccc(NC(=O)c3cncc(C#Cc4cnc5cccnn45)c3)cc2C(F)(F)F)CCN1 ; C[C@@H]1CN(Cc2ccc(NC(=O)c3cncc(C#Cc4cnc5cccnn45)c3)cc2C(F)(F)F)C[C@H](C)N1 ; C[C@@H]1C[C@H](C)CN(Cc2ccc(NC(=O)c3cncc(C#Cc4cnc5cccnn45)c3)cc2C(F)(F)F)C1]

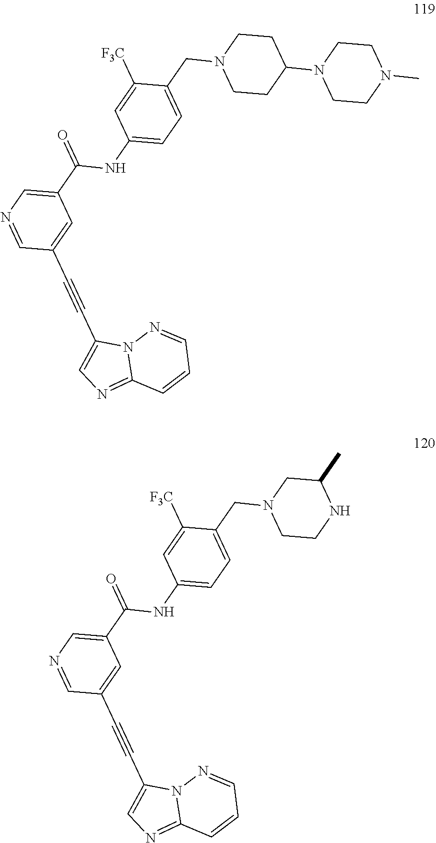 [SMILES: CN1CCN(C2CCN(Cc3ccc(NC(=O)c4cncc(C#Cc5cnc6cccnn56)c4)cc3C(F)(F)F)CC2)CC1 ; C[C@@H]1CN(Cc2ccc(NC(=O)c3cncc(C#Cc4cnc5cccnn45)c3)cc2C(F)(F)F)CCN1]

17. The compound of claim 1, wherein said compound is a compound of the following formula:

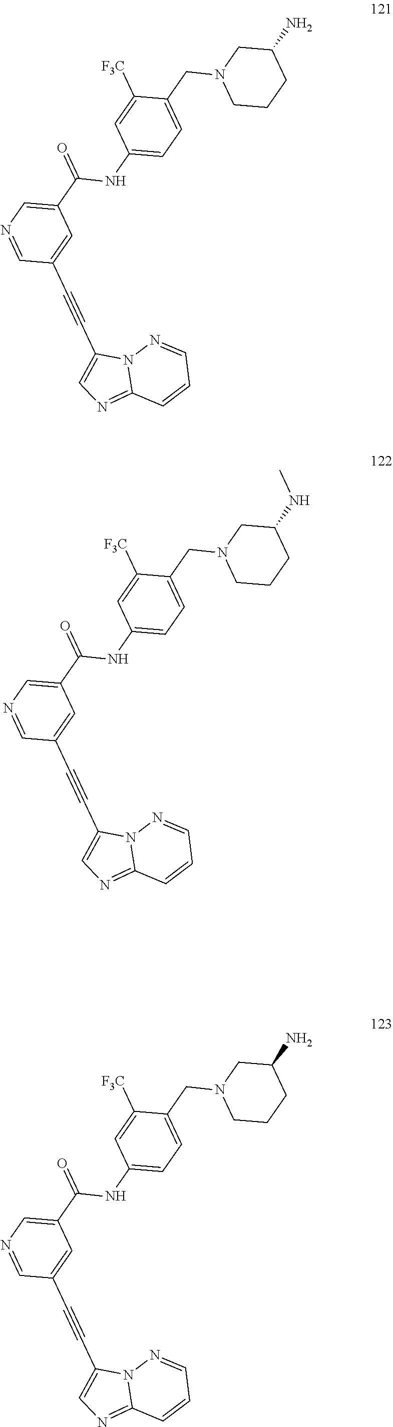 [SMILES: N[C@@H]1CCCN(Cc2ccc(NC(=O)c3cncc(C#Cc4cnc5cccnn45)c3)cc2C(F)(F)F)C1 ; CN[C@@H]1CCCN(Cc2ccc(NC(=O)c3cncc(C#Cc4cnc5cccnn45)c3)cc2C(F)(F)F)C1 ; N[C@H]1CCCN(Cc2ccc(NC(=O)c3cncc(C#Cc4cnc5cccnn45)c3)cc2C(F)(F)F)C1]

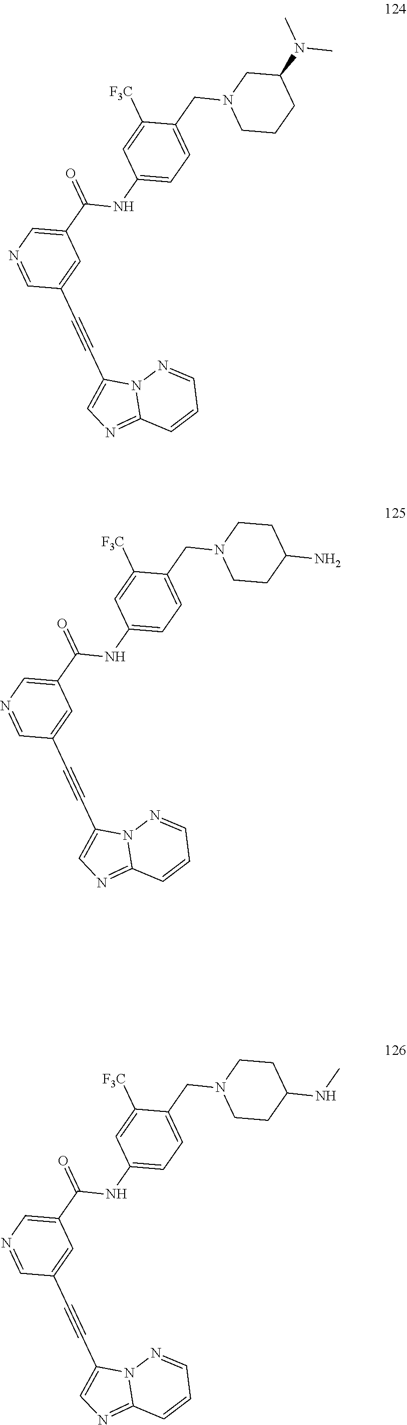 [SMILES: CN(C)[C@H]1CCCN(Cc2ccc(NC(=O)c3cncc(C#Cc4cnc5cccnn45)c3)cc2C(F)(F)F)C1 ; NC1CCN(Cc2ccc(NC(=O)c3cncc(C#Cc4cnc5cccnn45)c3)cc2C(F)(F)F)CC1 ; CNC1CCN(Cc2ccc(NC(=O)c3cncc(C#Cc4cnc5cccnn45)c3)cc2C(F)(F)F)CC1]

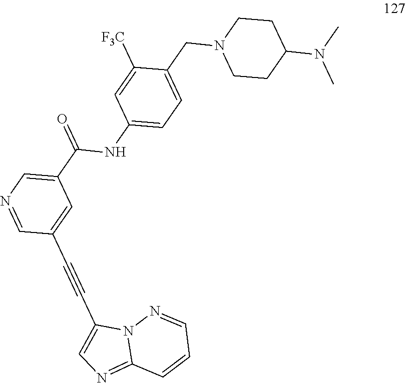 [SMILES: CN(C)C1CCN(Cc2ccc(NC(=O)c3cncc(C#Cc4cnc5cccnn45)c3)cc2C(F)(F)F)CC1]

18. The compound of claim 1, wherein said compound is a compound of the following formula:

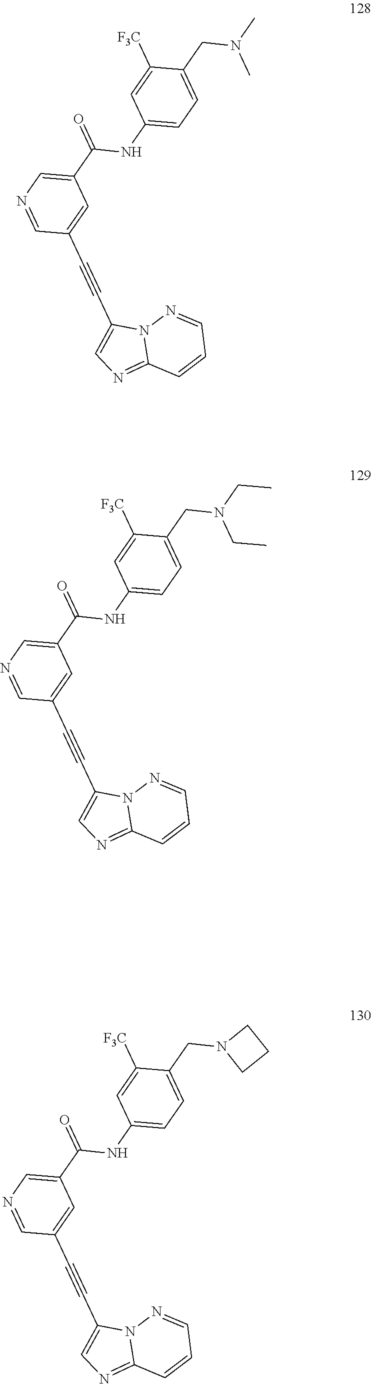 [SMILES: CN(C)Cc1ccc(NC(=O)c2cncc(C#Cc3cnc4cccnn34)c2)cc1C(F)(F)F ; CCN(CC)Cc1ccc(NC(=O)c2cncc(C#Cc3cnc4cccnn34)c2)cc1C(F)(F)F ; O=C(Nc1ccc(CN2CCC2)c(C(F)(F)F)c1)c1cncc(C#Cc2cnc3cccnn23)c1]

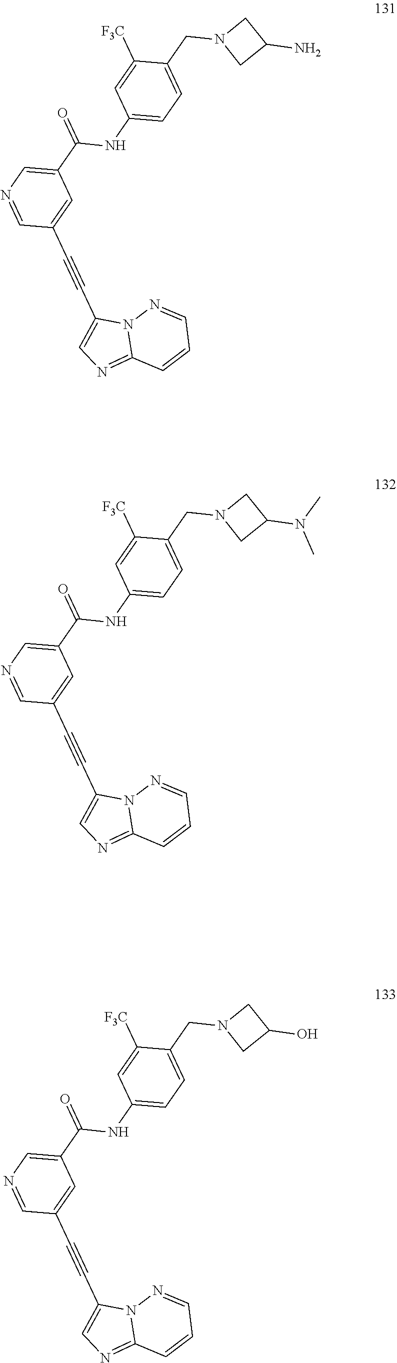 [SMILES: NC1CN(Cc2ccc(NC(=O)c3cncc(C#Cc4cnc5cccnn45)c3)cc2C(F)(F)F)C1 ; CN(C)C1CN(Cc2ccc(NC(=O)c3cncc(C#Cc4cnc5cccnn45)c3)cc2C(F)(F)F)C1 ; O=C(Nc1ccc(CN2CC(O)C2)c(C(F)(F)F)c1)c1cncc(C#Cc2cnc3cccnn23)c1]

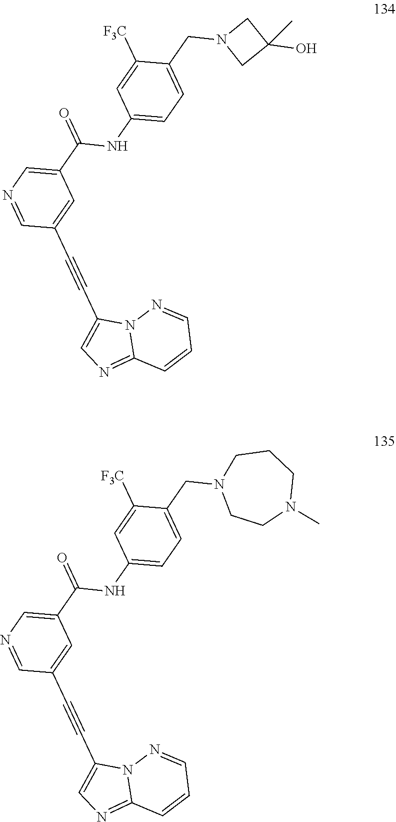 [SMILES: CC1(O)CN(Cc2ccc(NC(=O)c3cncc(C#Cc4cnc5cccnn45)c3)cc2C(F)(F)F)C1 ; CN1CCCN(Cc2ccc(NC(=O)c3cncc(C#Cc4cnc5cccnn45)c3)cc2C(F)(F)F)CC1]

19. The compound of claim 1, wherein said compound is a compound of the following formula:

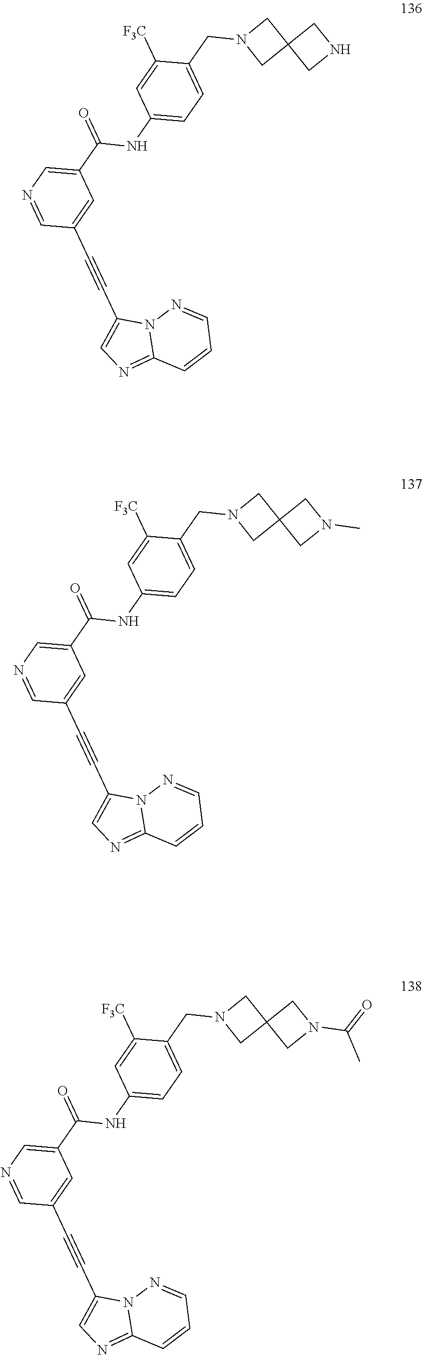 [SMILES: O=C(Nc1ccc(CN2CC3(CNC3)C2)c(C(F)(F)F)c1)c1cncc(C#Cc2cnc3cccnn23)c1 ; CN1CC2(C1)CN(Cc1ccc(NC(=O)c3cncc(C#Cc4cnc5cccnn45)c3)cc1C(F)(F)F)C2 ; CC(=O)N1CC2(CN(Cc3ccc(NC(=O)c4cncc(C#Cc5cnc6cccnn56)c4)cc3C(F)(F)F)C2)C1]

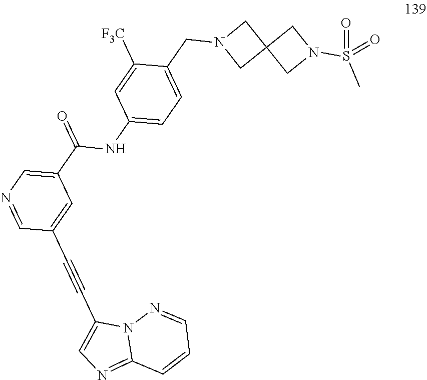 [SMILES: CS(=O)(=O)N1CC2(CN(Cc3ccc(NC(=O)c4cncc(C#Cc5cnc6cccnn56)c4)cc3C(F)(F)F)C2)C1]

20. The compound of claim 1, wherein said compound is a compound of the following formula:

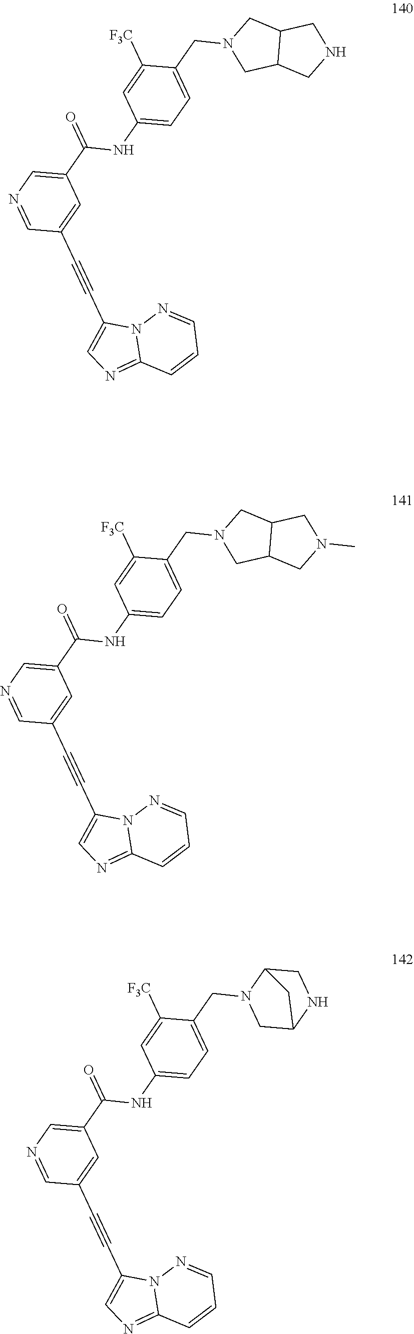 [SMILES: O=C(Nc1ccc(CN2CC3CNCC3C2)c(C(F)(F)F)c1)c1cncc(C#Cc2cnc3cccnn23)c1 ; CN1CC2CN(Cc3ccc(NC(=O)c4cncc(C#Cc5cnc6cccnn56)c4)cc3C(F)(F)F)CC2C1 ; O=C(Nc1ccc(CN2CC3CC2CN3)c(C(F)(F)F)c1)c1cncc(C#Cc2cnc3cccnn23)c1]

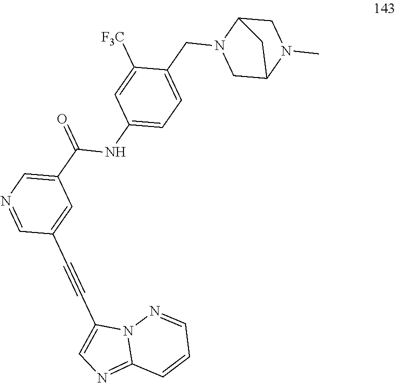 [SMILES: CN1CC2CC1CN2Cc1ccc(NC(=O)c2cncc(C#Cc3cnc4cccnn34)c2)cc1C(F)(F)F]

21. The compound of claim 1, wherein said compound is a compound of the following formula:

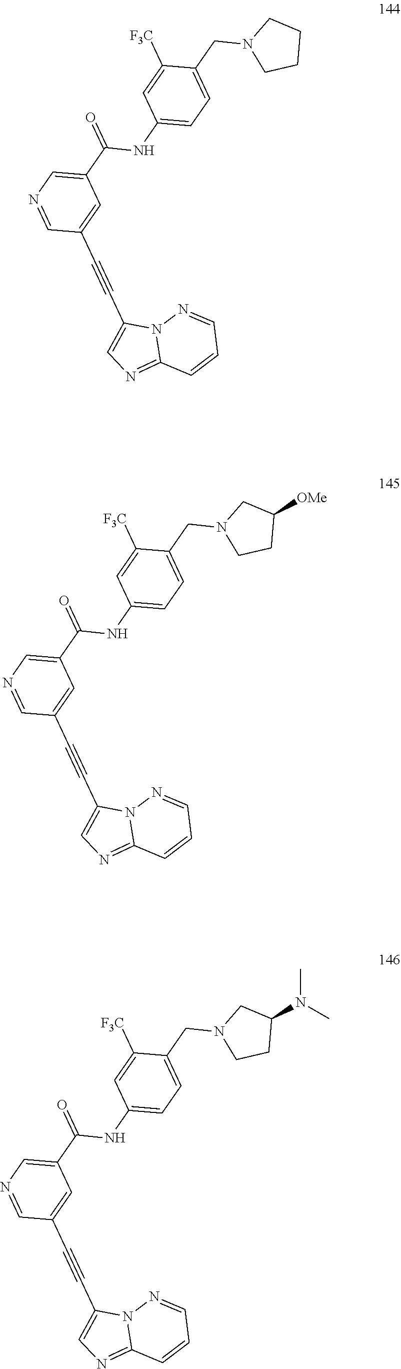 [SMILES: O=C(Nc1ccc(CN2CCCC2)c(C(F)(F)F)c1)c1cncc(C#Cc2cnc3cccnn23)c1 ; CO[C@H]1CCN(Cc2ccc(NC(=O)c3cncc(C#Cc4cnc5cccnn45)c3)cc2C(F)(F)F)C1 ; CN(C)[C@H]1CCN(Cc2ccc(NC(=O)c3cncc(C#Cc4cnc5cccnn45)c3)cc2C(F)(F)F)C1]

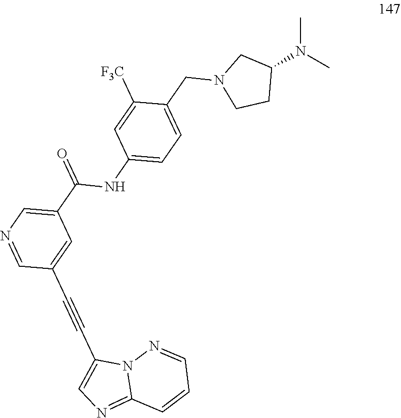 [SMILES: CN(C)[C@@H]1CCN(Cc2ccc(NC(=O)c3cncc(C#Cc4cnc5cccnn45)c3)cc2C(F)(F)F)C1]

22. The compound of claim 1, wherein said compound is a compound of the following formula:

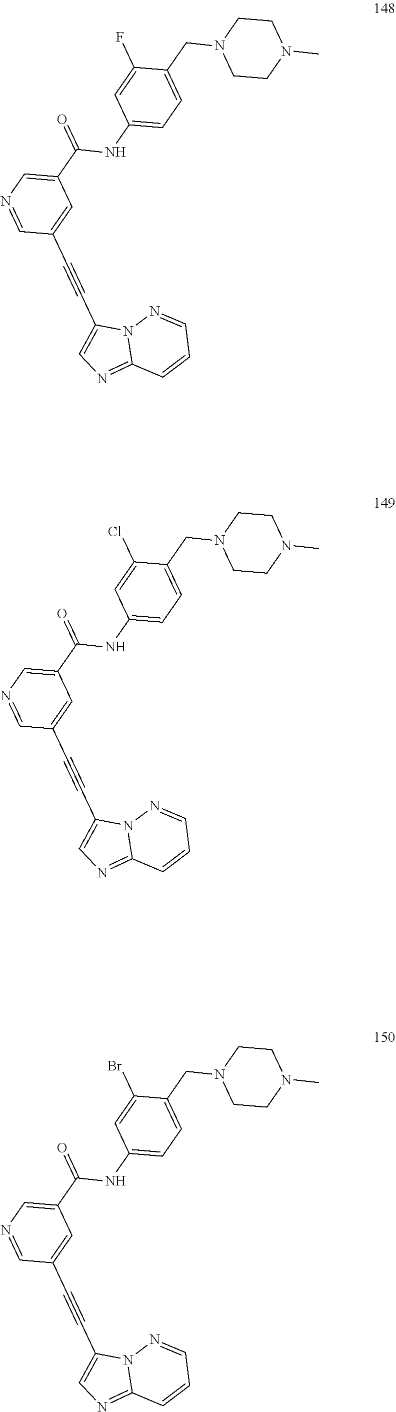 [SMILES: CN1CCN(Cc2ccc(NC(=O)c3cncc(C#Cc4cnc5cccnn45)c3)cc2F)CC1 ; CN1CCN(Cc2ccc(NC(=O)c3cncc(C#Cc4cnc5cccnn45)c3)cc2Cl)CC1 ; CN1CCN(Cc2ccc(NC(=O)c3cncc(C#Cc4cnc5cccnn45)c3)cc2Br)CC1]

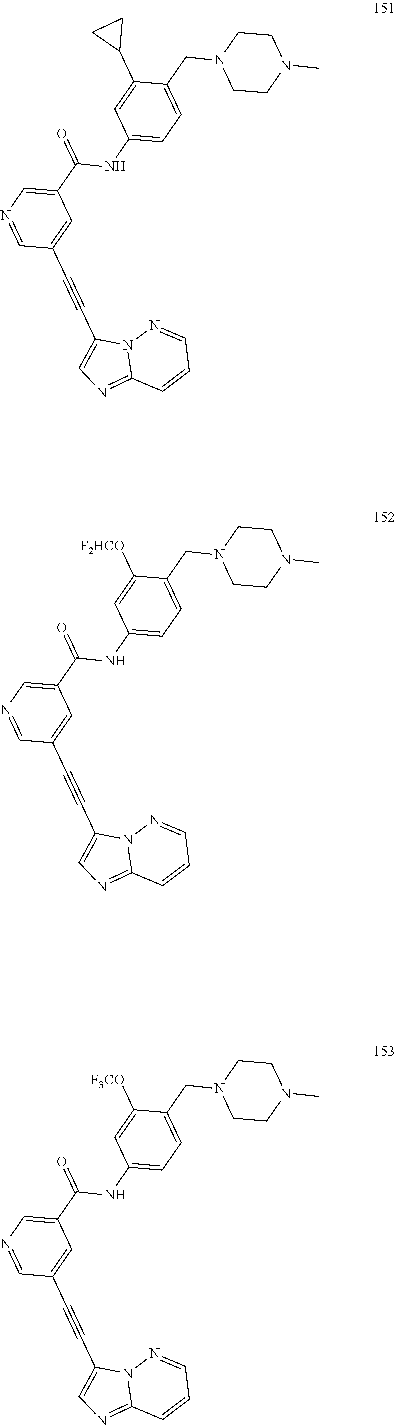 [SMILES: CN1CCN(Cc2ccc(NC(=O)c3cncc(C#Cc4cnc5cccnn45)c3)cc2C2CC2)CC1 ; CN1CCN(Cc2ccc(NC(=O)c3cncc(C#Cc4cnc5cccnn45)c3)cc2OC(F)F)CC1 ; CN1CCN(Cc2ccc(NC(=O)c3cncc(C#Cc4cnc5cccnn45)c3)cc2OC(F)(F)F)CC1]

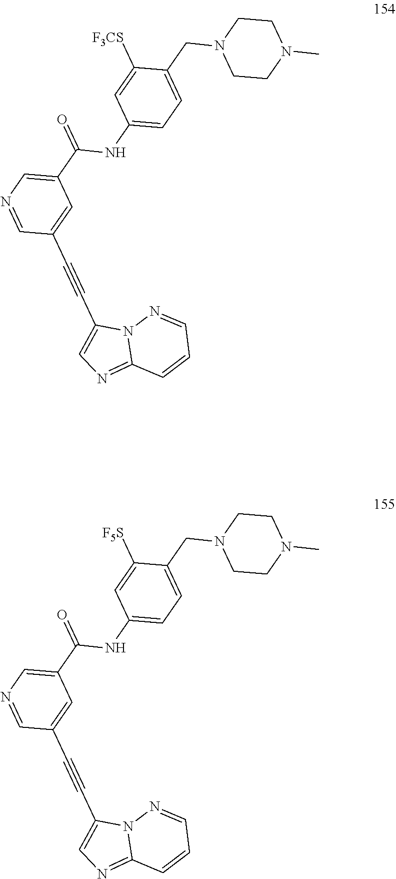

23. The compound of claim 1, wherein said compound is a compound of the following formula:

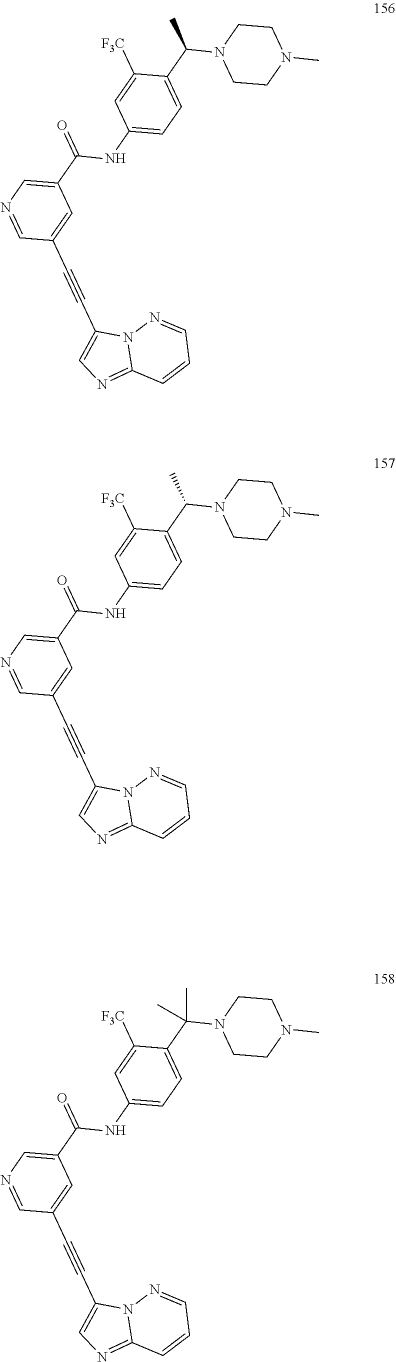 [SMILES: C[C@H](c1ccc(NC(=O)c2cncc(C#Cc3cnc4cccnn34)c2)cc1C(F)(F)F)N1CCN(C)CC1 ; C[C@@H](c1ccc(NC(=O)c2cncc(C#Cc3cnc4cccnn34)c2)cc1C(F)(F)F)N1CCN(C)CC1 ; CN1CCN(C(C)(C)c2ccc(NC(=O)c3cncc(C#Cc4cnc5cccnn45)c3)cc2C(F)(F)F)CC1]

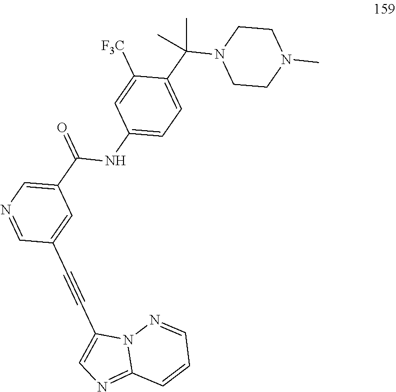 [SMILES: CN1CCN(C(C)(C)c2ccc(NC(=O)c3cncc(C#Cc4cnc5cccnn45)c3)cc2C(F)(F)F)CC1]

24. The compound of claim 1, wherein said compound is a compound of the following formula:

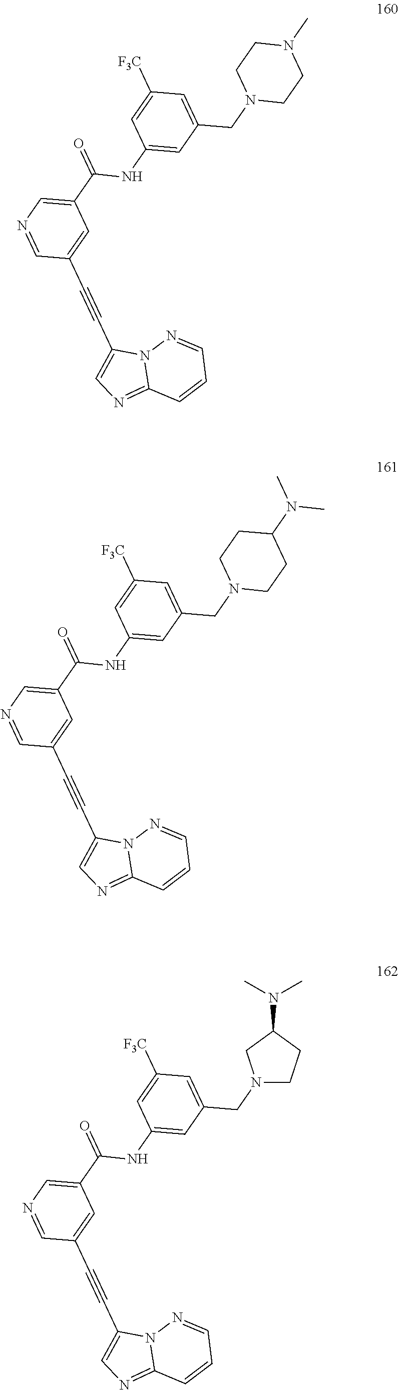 [SMILES: CN1CCN(Cc2cc(NC(=O)c3cncc(C#Cc4cnc5cccnn45)c3)cc(C(F)(F)F)c2)CC1 ; CN(C)C1CCN(Cc2cc(NC(=O)c3cncc(C#Cc4cnc5cccnn45)c3)cc(C(F)(F)F)c2)CC1 ; CN(C)[C@H]1CCN(Cc2cc(NC(=O)c3cncc(C#Cc4cnc5cccnn45)c3)cc(C(F)(F)F)c2)C1]

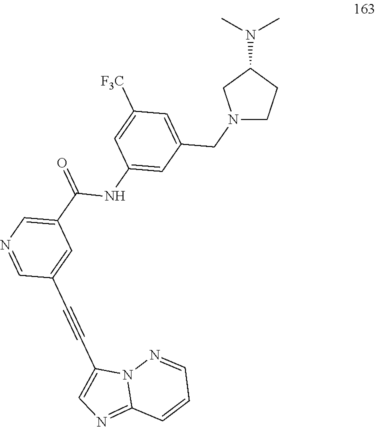 [SMILES: CN(C)[C@@H]1CCN(Cc2cc(NC(=O)c3cncc(C#Cc4cnc5cccnn45)c3)cc(C(F)(F)F)c2)C1]

25. The compound of claim 1, wherein said compound is a compound of the following formula:

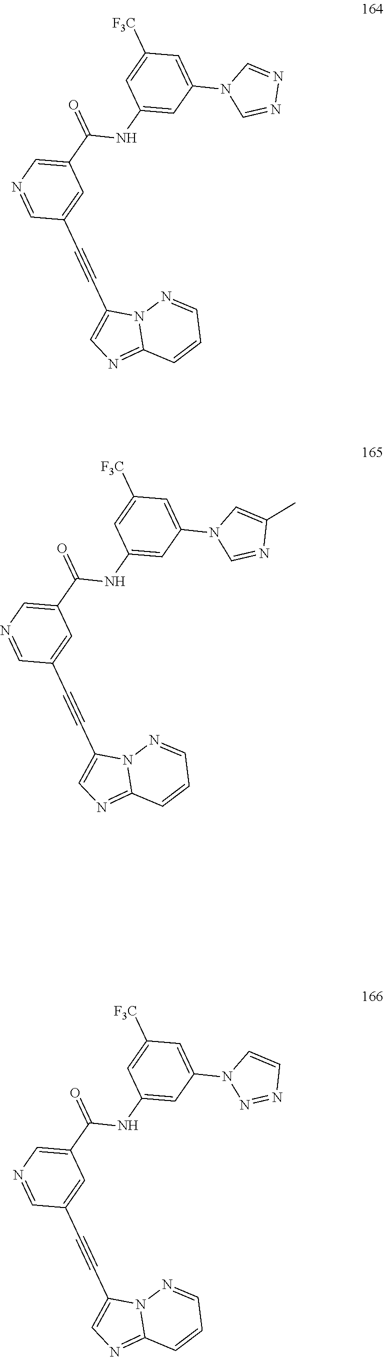 [SMILES: O=C(Nc1cc(-n2cnnc2)cc(C(F)(F)F)c1)c1cncc(C#Cc2cnc3cccnn23)c1 ; Cc1cn(-c2cc(NC(=O)c3cncc(C#Cc4cnc5cccnn45)c3)cc(C(F)(F)F)c2)cn1 ; O=C(Nc1cc(-n2ccnn2)cc(C(F)(F)F)c1)c1cncc(C#Cc2cnc3cccnn23)c1]

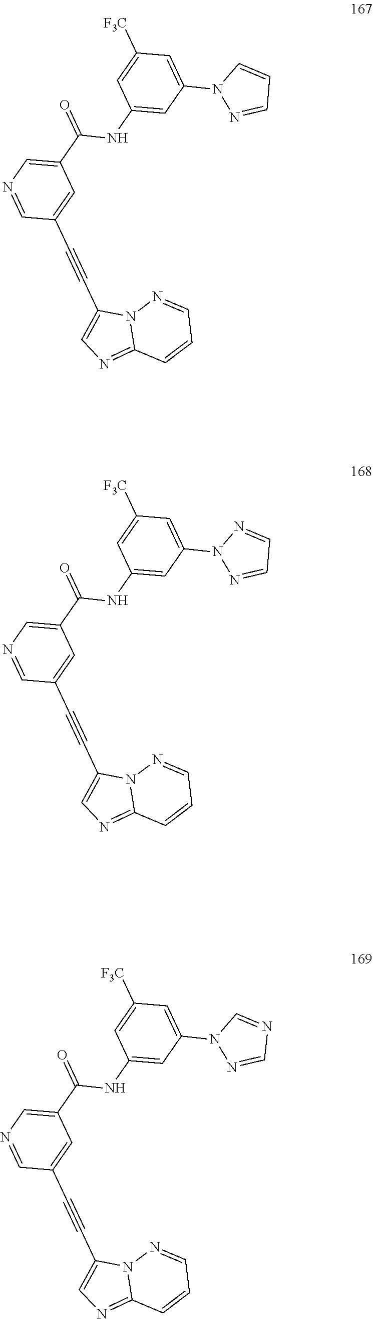 [SMILES: O=C(Nc1cc(-n2cccn2)cc(C(F)(F)F)c1)c1cncc(C#Cc2cnc3cccnn23)c1 ; O=C(Nc1cc(-n2nccn2)cc(C(F)(F)F)c1)c1cncc(C#Cc2cnc3cccnn23)c1 ; O=C(Nc1cc(-n2cncn2)cc(C(F)(F)F)c1)c1cncc(C#Cc2cnc3cccnn23)c1]

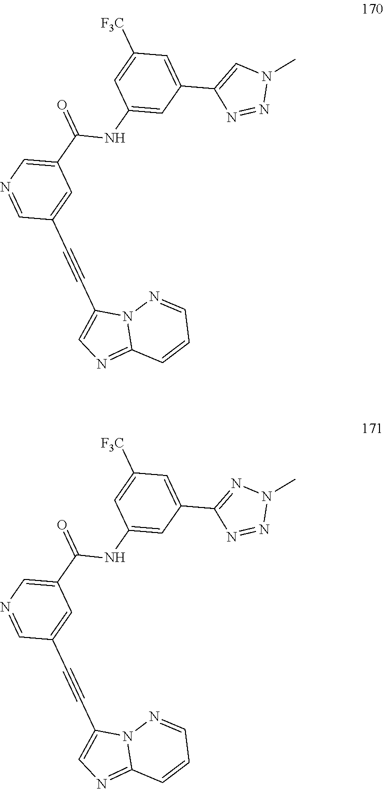 [SMILES: Cn1cc(-c2cc(NC(=O)c3cncc(C#Cc4cnc5cccnn45)c3)cc(C(F)(F)F)c2)nn1 ; Cn1nnc(-c2cc(NC(=O)c3cncc(C#Cc4cnc5cccnn45)c3)cc(C(F)(F)F)c2)n1]

26. The compound of claim 1, wherein said compound is a compound of the following formula:

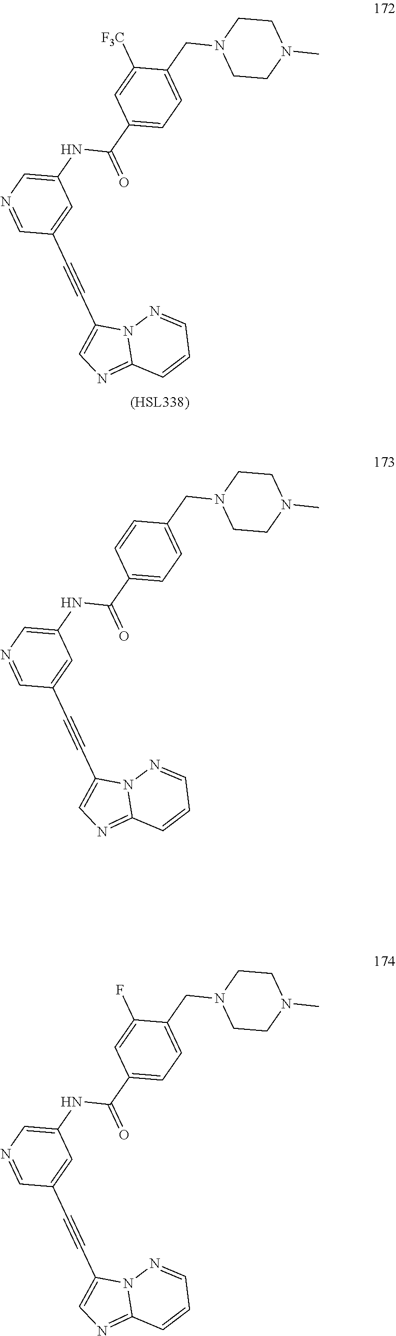 [SMILES: CN1CCN(Cc2ccc(C(=O)Nc3cncc(C#Cc4cnc5cccnn45)c3)cc2C(F)(F)F)CC1 ; CN1CCN(Cc2ccc(C(=O)Nc3cncc(C#Cc4cnc5cccnn45)c3)cc2)CC1 ; CN1CCN(Cc2ccc(C(=O)Nc3cncc(C#Cc4cnc5cccnn45)c3)cc2F)CC1]

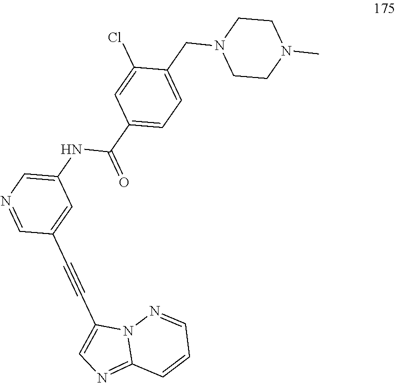 [SMILES: CN1CCN(Cc2ccc(C(=O)Nc3cncc(C#Cc4cnc5cccnn45)c3)cc2Cl)CC1]

27. The compound of claim 1, wherein said compound is a compound of the following formula:

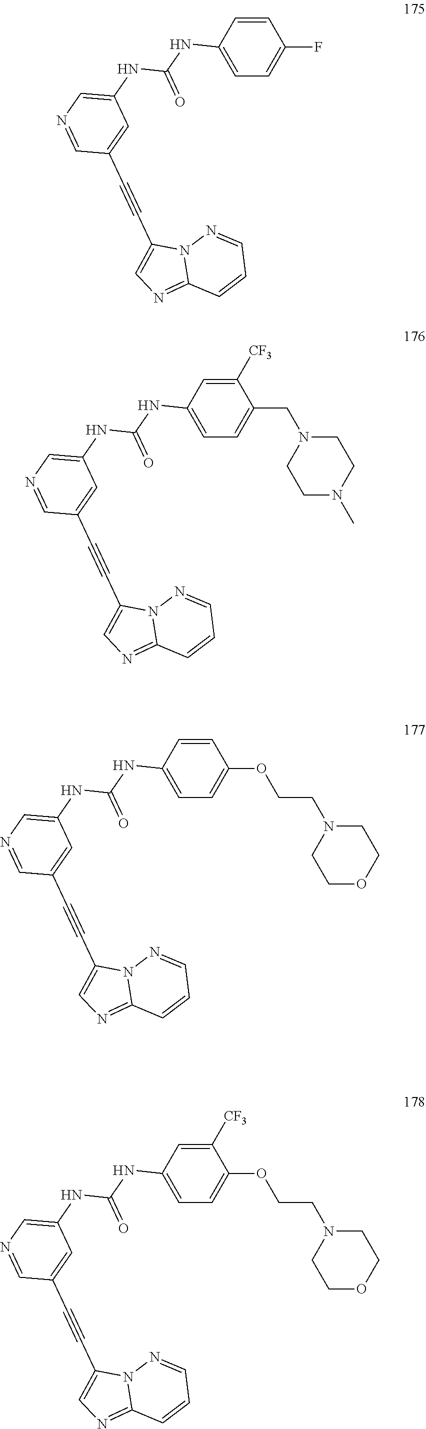 [SMILES: O=C(Nc1ccc(F)cc1)Nc1cncc(C#Cc2cnc3cccnn23)c1 ; CN1CCN(Cc2ccc(NC(=O)Nc3cncc(C#Cc4cnc5cccnn45)c3)cc2C(F)(F)F)CC1 ; O=C(Nc1ccc(OCCN2CCOCC2)cc1)Nc1cncc(C#Cc2cnc3cccnn23)c1 ; O=C(Nc1cncc(C#Cc2cnc3cccnn23)c1)Nc1ccc(OCCN2CCOCC2)c(C(F)(F)F)c1]

28. The compound of claim 1, wherein said compound is a compound of the following formula:

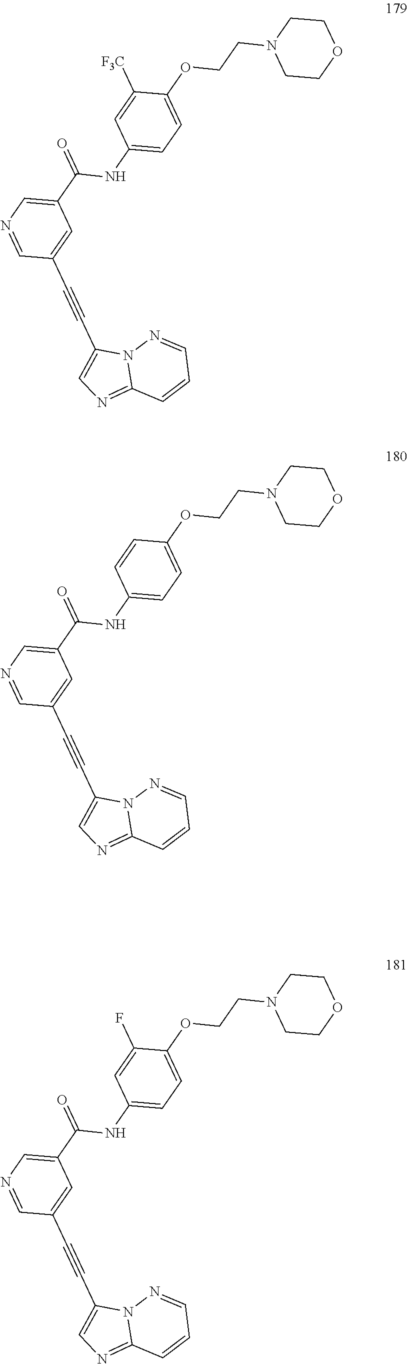 [SMILES: O=C(Nc1ccc(OCCN2CCOCC2)c(C(F)(F)F)c1)c1cncc(C#Cc2cnc3cccnn23)c1 ; O=C(Nc1ccc(OCCN2CCOCC2)cc1)c1cncc(C#Cc2cnc3cccnn23)c1 ; O=C(Nc1ccc(OCCN2CCOCC2)c(F)c1)c1cncc(C#Cc2cnc3cccnn23)c1]

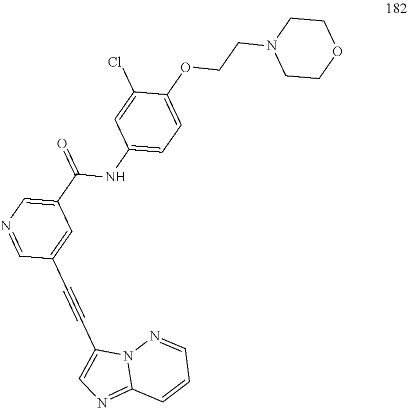 [SMILES: O=C(Nc1ccc(OCCN2CCOCC2)c(Cl)c1)c1cncc(C#Cc2cnc3cccnn23)c1]

29. The compound of claim 1, wherein said compound is a compound of the following formula:

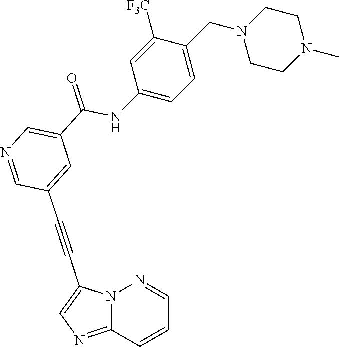 [SMILES: CN1CCN(Cc2ccc(NC(=O)c3cncc(C#Cc4cnc5cccnn45)c3)cc2C(F)(F)F)CC1]

30. A pharmaceutical composition comprising a compound of claim 1, or a pharmaceutically acceptable salt, N-oxide, hydrate, solvent, tautomer, or optical isomer thereof, and a pharmaceutically acceptable carrier or diluent.

31. A method of ameliorating cancer in a subject in need thereof, wherein the method comprises administering to the subject a therapeutically effective amount of a compound of claim 1, or a pharmaceutically acceptable salt, N-oxide, hydrate, solvate, tautomer, or optical isomer thereof, or a pharmaceutical composition comprising a compound of claim 1, or a pharmaceutically acceptable salt, N-oxide, hydrate, solvent, tautomer, or optical isomer thereof, and a pharmaceutically acceptable carrier or diluent.

32. The method of claim 31, wherein said cancer is selected from the group consisting of acute myeloid leukemia, chronic myeloid leukemia, ovarian cancer, cervical cancer, pancreatic cancer, breast cancer, brain cancer, skin cancer, lung cancer, prostate cancer, Lymphoma, Leukemia, colon cancer, head cancer, neck cancer, thyroid cancer, kidney cancer, liver cancer, and stomach cancer.

33. A method of ameliorating a disease or a disorder associated with protein kinase in a subject in need thereof, wherein the method comprises administering to the subject a therapeutically effective amount of a claim 1, or a pharmaceutically acceptable salt, N-oxide, hydrate, solvate, tautomer, or optical isomer thereof, or a pharmaceutical composition comprising a compound of claim 1, or a pharmaceutically acceptable salt, N-oxide, hydrate, solvent, tautomer, or optical isomer thereof, and a pharmaceutically acceptable carrier or diluent.

34. The method of claim 33, wherein said protein kinase is Abl, Abl2, AFK, ALK, AMPK_group, ATM, ATR, Aurora A, Aurora B, Axl, BCKDK, BLK, BMPR1B, BMX, Brk, BRSK1, BTK, CaM-Klalpha, CaM-Kllalpha, CaMKK_group, CaM-KIV, CaM-KKalpha, CaM-KKbeta, CCDPK, CCRK, CDK1, CDK11, CDK2, CDK4, CDK5, CDK6, CDK7, CDK9, CDK_group, CDPK, Chak1, CHK1, CHK2, CK1 alpha, CK1 delta, CK1 epsilon, CK1_group, CK2 alpha, CK2_beta, CK2_group, CLK1, CSF1R, Csk, DAPK1, DAPK2, DAPK3, DAPK_group, DCAMKL1, DMPK_group, DNA-PK, DYRK1A, DYRK1B, DYRK2, DYRK3, eEF2K, Eg3 kinase, EGFR, EIF2AK2, EphA2, EphA3, EphA4, EphA8, EphB1, EphB2, EphB3, EphB5, ErbB2, FAK, Fer, Fes, FGFR1, FGFR3, FGFR4, FGFR_group, Fgr, FLT1, FLT3, FLT4, Fyn, GRK-1, GRK-2, GRK-3, GRK-4, GRK-5, GRK-6, GRK_group, GSK-3alpha, GSK-3beta, GSK-3_group, HCK, HIPK2, HIPK3, HRI, ICK, IGF1R, IKK-alpha, IKK-beta, IKK-epsilon ILK, InsR, IPL1, IRAK1, IRAK4, ITK, JAK1, JAK2, JAK3, JAK_group, JNK_group, KDR, KIS, Kit, KSR1, Lck, LIMK1, LIMK2, LKB1, LOK, Lyn, MAP2K1, MAP2K2, MAP2K3, MAP2K4, MAP2K6, MAP2K7, MAPK2_group, MAP3K1, MAP3K11, MAP3K14, MAP3K5, MAP3K7, MAP3K8, MAPK3_group, MAP4K1, MAP4K2, MAP4K4, MAPK1, MAPK10, MAPK11, MAPK12, MAPK13, MAPK14, MAPK3, MAPK4, MAPK6, MAPK7, MAPK8, MAPK9, MAPK_group, MAPKAPK2, MARK_group, Mer, Met, MHCK, MLCK_group, Mnk1, Mnk2, MOS, MRCKα, MST1, MST3, mTOR, NDR1, NDR2, NEK1, NEK2, NEK6, NEK9, NEK_group, NLK, NuaK1, p37 kinase, p38_group, p70S6K, p70S6Kb, P70S6K_group, PAK1, PAK2, PAK3, PAK5, PAK6, PAK_group, PASK, P-CIP2, PCTAIRE1, PDGFR alpha, PDGFR beta, PDGFR_group, PDHK1, PDHK2, PDHK3, PDHK4, PDK-1, PDK-2, PDK_group, PHK_group, PIK3CA, PIK3CB, PIK3CD, PIK3CG, Pim-1, PKA alpha, Pka_group, PKB beta, PKB_group, PKC alpha, PKC beta, PKC delta, PKC epsilon, PKC eta, PKC gamma, PKC iota, PKC theta, PKC zeta, PKC_group, PKD1, PKD2, PKD3, PKG1/cGK-I, PKG2/cGK-II, PKG2/cGK_group, PKN1, PLK1, PLK2, PLK3, PRP4, PYK2, RAF1, Ret, ROCK1, ROCK2, Ron, RPL10, RSK-1, RSK-2, RSK-3, RSK-5, SDK1, SGK_group, SIK, Sky, Src, Src_group, STLK3, Syk, TBK1, Tec, TESK1, TESK2, TGFbR1, TGFbR2, Tie1, Tie2, Titin kinase, TNK2, TRKA, TRKB, tropomyosin kinase, TSSK3, TXK, Tyk2, TYK2, VRK1, Wee1, Wnk1, WNK1, Yes, or ZAP70.

35. The method of claim 33, wherein said disease or disorder is cancer, diabetes, malaria, viral infections, cardiovascular and hypertension, CNS and neurodegeneration, osteoporosis, pulmonary fibrosis, retinitis pigmentosis, Wet macular degeneration, Duchenne muscular dystrophy, diabetic eye disease, inflammation and autoimmune, or allergy.

36. A treatment delivery apparatus comprising a device and at least one dosage of a composition contained in the device, wherein the composition is a therapeutically effective amount of a compound or a combination of compounds according to claim 1, or a pharmaceutically acceptable salt, N-oxide, hydrate, solvate, tautomer, or optical isomer thereof, or a pharmaceutical composition comprising a compound of claim 1, or a pharmaceutically acceptable salt, N-oxide, hydrate, solvent, tautomer, or optical isomer thereof, and a pharmaceutically acceptable carrier or diluent.

37. A treatment delivery apparatus comprising a delivery mechanism selected from the group consisting of: capsules, polypills, tablets, transdermal patches, dietary supplements, or a combination thereof; and at least one dosage of a composition contained in the delivery mechanism, wherein the composition is a therapeutically effective amount of a compound or a combination of compounds according to claim 1, or a pharmaceutically acceptable salt, N-oxide, hydrate, solvate, tautomer, or optical isomer thereof, or a pharmaceutical composition comprising a compound of claim 1, or a pharmaceutically acceptable salt, N-oxide, hydrate, solvent, tautomer, or optical isomer thereof, and a pharmaceutically acceptable carrier or diluent.

38. The treatment delivery system according to claim 36 wherein the delivery apparatus allows for the controlled or delayed release of the composition contained in the delivery system.

39. The treatment delivery system according to claim 37 wherein the delivery apparatus allows for the controlled or delayed release of the composition contained in the delivery system.
